(19) United States
(12) Patent Application Publication
(10) Pub. No.: US 2026/0308145 A1
(43) Pub. Date: Oct. 8, 2026

(54) CDK2 INHIBITORS AND METHODS OF USING SAME

(71) Applicants: Shenzhen lonova Life Science Co., Ltd., Shenzhen (CN); Foshan lonova Biotherapeutics Co., Inc., Foshan (CN); Guangdong Touchstone Translational Research Institute Co., Ltd., Foshan (CN)

(72) Inventors: Hongjian Yang, Shenzhen (CN); Yexing Cao, Shenzhen (CN); Chang Bai, Shenzhen (CN); Jialiang Qin, Shenzhen (CN); Jing Su, Scotch Plains, NJ (US)

(21) Appl. No.: 19/477,773

(22) PCT Filed: Apr. 23, 2024
(86) PCT No.: PCT/CN2024/089320

§ 371 Date: Oct. 22, 2025

Related U.S. Application Data
(60) Provisional application No. 63/501,034, filed on May 9, 2023.
(60) Provisional application No. 63/615,863, filed on Dec. 29, 2023.

(30) Foreign Application Priority Data
Apr. 24, 2023 (WO) PCT/CN2023/090329
Oct. 30, 2023 (WO) PCT/CN2023/127659

(51) Int. Cl. C07D 401/14; A61K 31/506; A61K 31/635; A61K 45/06; C07B 59/00; C07D 403/04; C07D 403/14; C07D 405/14; C07D 417/14

(52) U.S. Cl. CPC C07D 401/14; A61K 31/506; A61K 31/635; A61K 45/06; C07B 59/002; C07D 403/04; C07D 403/14; C07D 405/14; C07D 417/14

(57) ABSTRACT

The present invention provides novel compounds of Formula (I) as CDK2 inhibitors, which are useful for treating diseases or conditions mediated by CDK2, in particular, cancers and other diseases caused by abnormal cell proliferation. Preparation methods, pharmaceutical compositions and methods of use are also included.

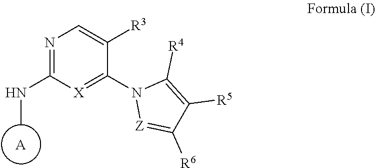

### CROSS REFERENCE TO RELATED APPLICATIONS

This application claims priority to PCT Application No. PCT/CN2023/090329, filed on Apr. 24, 2023; U.S. Application No. 63/501,034, filed on May 9, 2023; PCT Application No. PCT/CN2023/127659, filed on Oct. 30, 2023; and U.S. Application No. 63/615,863, filed on Dec. 29, 2023, the contents of all of which are incorporated herein by reference in their entirety.

### BACKGROUND OF THE INVENTION

Cyclin-dependent kinases (CDKs) are serine/threonine kinases whose activity depends on a regulatory subunit-a cyclin. Without cyclin, CDK has little kinase activity; only the cyclin-CDK complex is an active kinase, but its activity can be further modulated by phosphorylation and other binding proteins. Based on the sequence of the kinase domain, CDKs belong to the CMGC group of kinases, along with mitogen-activated protein kinases (MAPKs), glycogen synthase kinase-3 beta (Gsk3β), members of the dual-specificity tyrosine-regulated kinase (DYRK) family and CDK-like kinases (see, Genome Biol. 2014; 15(6): 122). The CDKs play important roles in many aspects of cell growth, proliferation, and transcriptional regulation in response to extracellular and intracellular signals. The evolutionary relationships between these CDK subfamilies have been identified and indicate that the CDK subfamilies can be divided into subfamilies that directly or indirectly regulate the cell cycle (CDKs1-6, 11 and 14-18) or transcription (CDKs7-13, 19 and 20) (see, Pharmacol Ther. 2017 May; 173: 83-105).

The essential roles of CDKs in regulation of cell division, gene transcription and other critical biological processes make them attractive pharmacological targets. Over the past two decades there has been a great deal of interest in the development of CDK inhibitors. This attention initially stemmed from observations that different CDK isoforms have key roles in cancer cell proliferation through dysregulation of the cell cycle, a hallmark feature of cancer. The cell cycle is a four-stage process in which the cell increases in size (gap 1, or G1, stage), copies its DNA (synthesis, or S, stage), prepares to divide (gap 2, or G2, stage), and divides (mitosis, or M, stage). At each checkpoint, a variety of proteins engage in a series of carefully coordinated biochemical reactions, which ensure that cell division occurs after sufficient growth and DNA replication and under favorable conditions. CDKs are major drivers of cell cycle regulation mechanisms, which promote DNA synthesis and mitosis by phosphorylation of key substrates. Consequently, the abnormal activation of CDKs promotes dysregulation of the cell cycle and uncontrolled proliferation of cells, leading to the development of malignant cancer. Now, CDKs have been shown to regulate other processes, particularly various aspects of transcription (see, Pharmacol Ther. 2017 May; 173: 83-105; Transcription. 2017; 8(2): 81-90). Thus, CDK inhibitors have therapeutic potential for treating various diseases caused by CDK abnormality, such as cancer, autoimmune diseases, cardiovascular diseases, neurodegenerative disorders, and infectious diseases, among others.

Efforts aimed at targeting CDK hyperactivity in human cancers began through the purification of compounds from natural sources. Determination of their molecular nature and further biochemical and structural studies enabled a better understanding of their inhibitory potential and provided a way for structure-guided, rational design of CDK inhibitors. The first generation CDK inhibitors developed were relatively nonspecific, referred to as pan-CDK inhibitors, and suffered certain limitations associated with toxic side-effects, which prompted the development of second-generation CDK inhibitors with a narrower spectrum of selectivity, offering promises of greater efficacy and reduced side effects. CDK4/6 inhibitors are the first ones that were approved by FDA for clinical treatment. These inhibitors specifically inhibit CDK4/6 and show limited toxicity to normal cells. The FDA-approved CDK4/6 inhibitor drugs include Palbociclib (Pfizer), Ribociclib (Novartis), Abemaciclib (Eli Lilly), and Trilaciclib (G1 Therapeutics) (see, J. Med. Chem. 2022, 65, 9, 6356-6389).

Like other family members, CDK2 activation requires binding to a cyclin binding partner, either cyclin E or cyclin A. Activation of CDK2 results in the phosphorylation of proteins involved in the initiation DNA synthesis and progression of the cell cycle from G1 through S phase (Cell Cycle 9:22, 4533-4541; Nov. 15, 2010). In the G1/S phase, CDK4 and/or CDK6 in a complex with cyclin D initially phosphorylate the retinoblastoma (Rb) protein. Following the association of E-type cyclins (Cyclin E1 and Cyclin E2, CCNE1 and CCNE2 genes) with CDK2, CDK2/cyclin E complex completes the phosphorylation of Rb, which releases and activates the E2F family transcriptional activity, driving cells to the S phase. Besides the phosphorylation of the Rb protein, the activity of the CDK2/cyclin E complex is required for MCM (mini-chromosome maintenance) proteins essential for the initiation of replication. In late S phase, A-type cyclins that are associated with CDK2 or CDK1 phosphorylate their substrates, including MCMs, Cdc7 or ribonucleotide reductase R2, to control the cell cycle transition from S to G2 (Oncogene. 2016 Mar. 3; 35(9): 1170-1179).

Oncogenic activation of CDK2/cyclin complex is associated with abnormal regulation of the cell-cycle, causing replication stress and DNA damage, and thus contributing to human carcinogenesis. Therefore, targeting oncogenic activity of CDK2/cyclin complex may represent an attractive therapeutic strategy for cancer treatment. Despite significant efforts, there are no FDA approved agents targeting CDK2 to date. In this regard, the present invention provides novel compounds as selective CDK2 inhibitors, preparation methods thereof and use thereof in treating diseases mediated by CDK2, such as cancer or other proliferative diseases or conditions.

### BRIEF SUMMARY OF THE INVENTION

The present invention is related to compounds of Formula (I) and pharmaceutically acceptable salts, tautomers, or stereoisomers thereof. Such compounds can inhibit activities of CDKs, in particular, CDK2. In some embodiments, the compounds can selectively inhibit the activity of CDK2, thereby presenting good clinical prospects. Also provided are pharmaceutical compositions and methods of treating diseases mediated by CDK2 with these compounds.

In one aspect, the invention provides a compound of Formula (I) or a pharmaceutically acceptable salt, tautomer, or stereoisomer thereof,

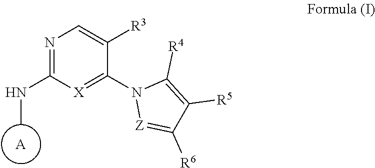

wherein:
X is CH or N;
Z is CH or N;
ring A is aryl, heteroaryl, cycloalkyl, heterocyclyl, or aryl-fused heterocyclyl; each of which is optionally and independently substituted with one R¹ and one to four R²; wherein a carbon atom of the aryl-fused heterocyclyl is optionally substituted with one oxo (═O), and the heteroatom of the aryl-fused heterocyclyl is optionally substituted with one or two oxo;
R¹ is R¹⁰—S(O)₂—, R¹⁰—S(O)₂—NH— or —SF₅;
each R² is independently H, alkyl, —CN, cyanoalkyl, amino, alkylamino, dialkylamino, —NO₂, halo, haloalkyl, hydroxyl, hydroxyalkyl, alkoxyl, haloalkoxy, (haloalkoxy)alkyl, alkoxyalkyl, cycloalkyl or heterocyclyl; wherein the cycloalkyl or heterocyclyl is optionally substituted with one or more R⁷;
R³ is H, halo, alkyl, haloalkyl, hydroxyl, hydroxyalkyl, alkoxyl, alkoxyalkyl, alkenyl, alkynyl, —CN or cyanoalkyl;
R⁴ and R⁶ are each independently H, alkyl, halo, haloalkyl, hydroxyl, hydroxyalkyl, alkoxyl, haloalkoxy, (haloalkoxy)alkyl, alkoxyalkyl, amino, alkylamino, dialkylamino, -alk-N—R⁹R⁹, —CN, cyanoalkyl, cycloalkyl or heterocyclyl; wherein the cycloalkyl or heterocyclyl is optionally substituted with one or more R⁷;
R^{S} is H, alkyl, halo, haloalkyl, hydroxyl, hydroxyalkyl, alkoxyl, haloalkoxy, (haloalkoxy)alkyl, alkoxyalkyl, —CN, cyanoalkyl, amino, alkylamino, dialkylamino, -alk-N—R⁹R⁹, aryl, heteroaryl, cycloalkyl, cycloalkenyl, heterocyclyl, -alk-cycloalkyl, -alk-heterocyclyl, —O— cycloalkyl, or —O-heterocyclyl, wherein the aryl, heteroaryl, cycloalkyl, cycloalkenyl, heterocyclyl, -alk-cycloalkyl, -alk-heterocyclyl, —O-cycloalkyl or —O-heterocyclyl is optionally substituted with one or more R⁷;
each R⁷ is independently alkyl, halo, haloalkyl, hydroxyl, hydroxyalkyl, alkoxyl, haloalkoxy, (haloalkoxy)alkyl, alkoxyalkyl, amino, alkylamino, dialkylamino, —CN, cyanoalkyl, oxo (═O), R⁸—S(O)₂—, R⁸—O—C(═O)—, R⁸—C(═O)—O—, R⁸—C(═O)—, cycloalkyl or heterocyclyl; wherein the cycloalkyl or heterocyclyl is optionally further substituted with one or more R⁷;
each R⁸ is independently H, alkyl or —NR⁹R⁹;
each R⁹ is independently H, cycloalkyl, -alk-cycloalkyl, heterocyclyl, -alk-heterocyclyl, or alkyl;
R¹⁰ is H, alkyl, haloalkyl, hydroxyalkyl, alkoxyalkyl, cycloalkyl, heterocyclyl or —NR¹R¹; wherein the cycloalkyl or heterocyclyl is optionally substituted with one or more R⁷;
each R¹¹ is independently H, alkyl, cycloalkyl, heterocyclyl, -alk-cycloalkyl, -alk-heterocyclyl, hydroxyalkyl, alkoxyalkyl, -alk-O—R¹², -alk-C(O)—OR⁹, or -alk-N—R⁹R⁹; wherein the cycloalkyl, -alk-cycloalkyl, -alk-heterocyclyl or heterocyclyl is optionally substituted with one or more R⁷;
each R¹² is independently H, alkyl, cycloalkyl, haloalkyl, or heterocyclyl;
the heteroaryl and heterocyclyl in each occurrence independently contain one or more ring-forming heteroatoms each of which is independently oxygen, sulfur or nitrogen;
the heterocyclyl in each occurrence is saturated or partially unsaturated; and
any H in each alkyl is optionally replaced with deuterium (D).

In some preferred embodiments, the compound of Formula (I) is of Formula (II):

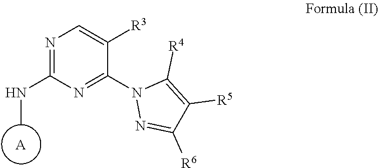

in which Ring A, R¹, R², R³, R⁴, R⁵ and R⁶ are the same as defined above.

In some embodiments, ring A is:

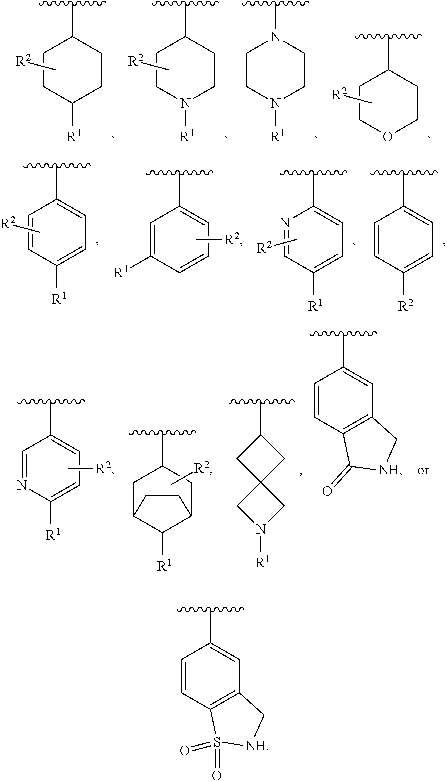

The hydroxyalkyl can be linear or branched; the cyanoalkyl can be linear or branched; and the cycloalkyl, cycloalkenyl or heterocyclyl can be monocyclic or bicyclic.

In some other embodiments, the compound of Formula (I) is of Formula (IIa), (IIb), (IIc), (IId) or (IIe):

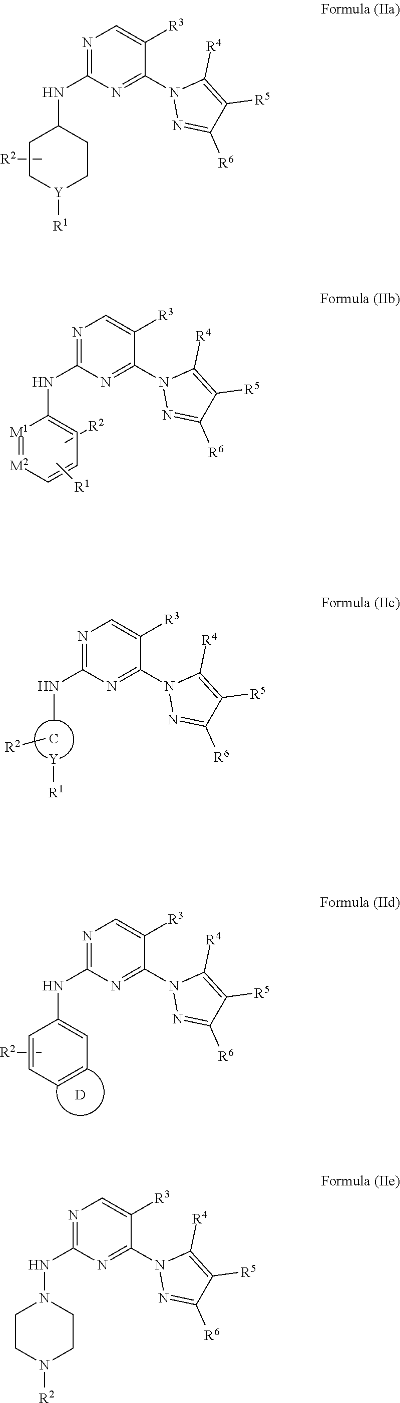

wherein
ring C is a bridged or spiro bicyclic heterocycloalkyl;
ring D is a 4-8 membered cycloalkyl or heterocyclyl fused to the adjacent aryl; wherein the cycloalkyl or heterocycloalkyl is optionally substituted with one or two oxo;
Y is N, O or CH; with the provision that when Y is O, R¹ is absent;
M¹ and M² are each independently N or CH; and
R¹, R², R³, R⁴, R⁵ and R⁶ are the same as defined above.

In some embodiments, in Formula (IIb), M¹ and M² are CH.

In some embodiments, R¹ is NHR¹¹—S(O)₂—, heterocyclyl-S(O)₂—, C₁₋₆alkyl-S(O)₂—, (C₁₋₆ alkyl)₂-N—S(O)₂—, (deuterated C₁₋₆alkyl)₂-N—S(O)₂—, C₁₋₆alkyl-S(O)₂—NH—, (deuterated C₁₋₆alkyl)-HN—S(O)₂—, or —SF₅.

In some embodiments, halo is F, Cl, or Br.

In some embodiments, R² is H, halo, haloalkyl, hydroxyl, alkyl, cycloalkyl or heterocyclyl; wherein the cycloalkyl or heterocyclyl is optionally substituted with one or more R⁷.

In some embodiments, R³ is H, halo, alkyl, haloalkyl, alkynyl, —CN, or cyanoalkyl.

In some embodiments, the compound of Formula (I) is of Formula (III):

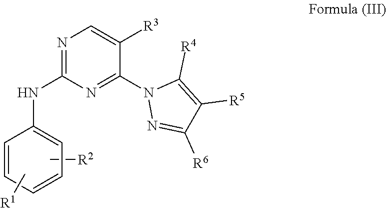

wherein R¹, R², R³, R⁴, R⁵ and R⁶ are the same as defined above.

Without limitation, exemplary compounds according to the present invention include:

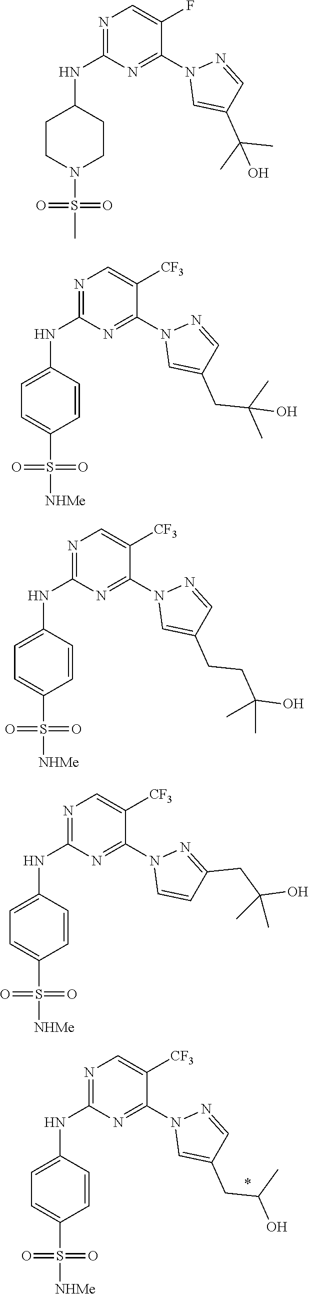

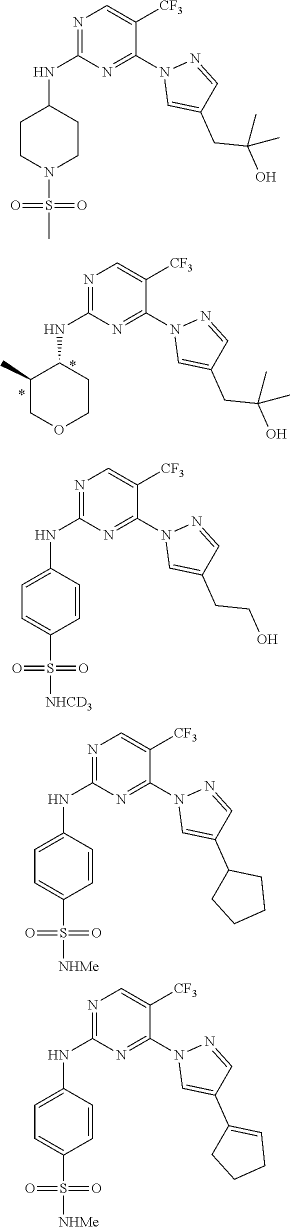

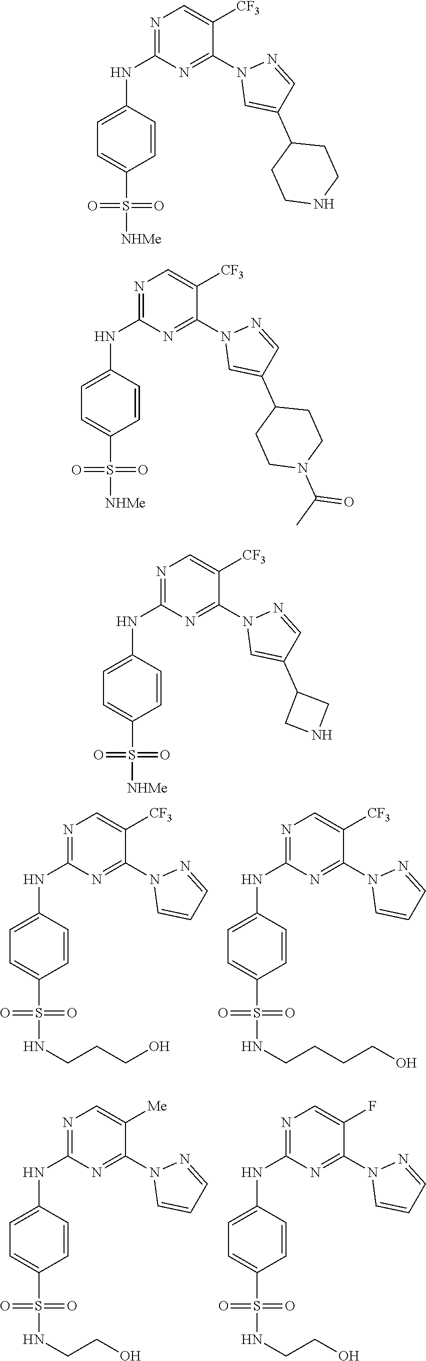

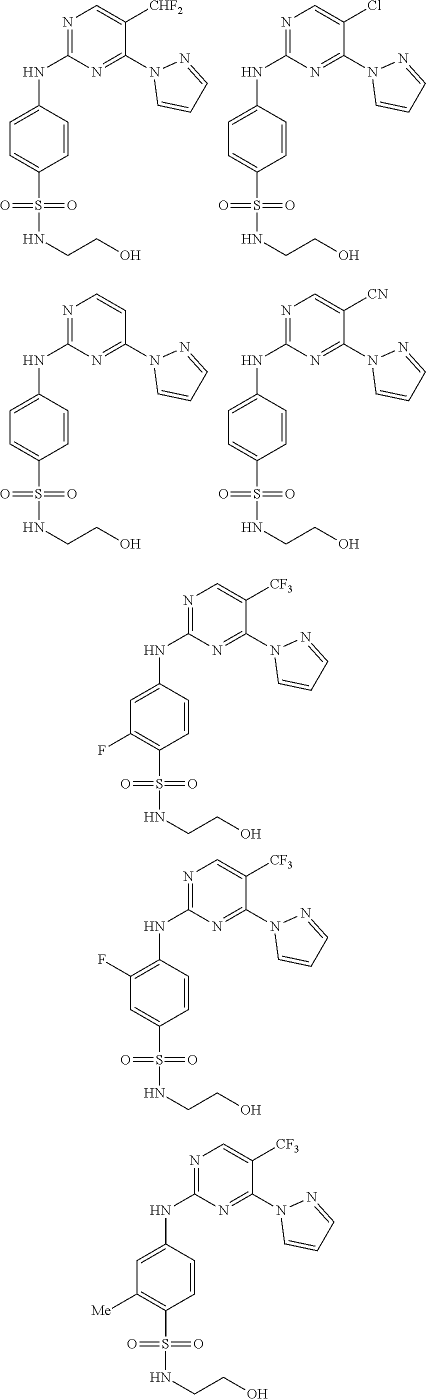

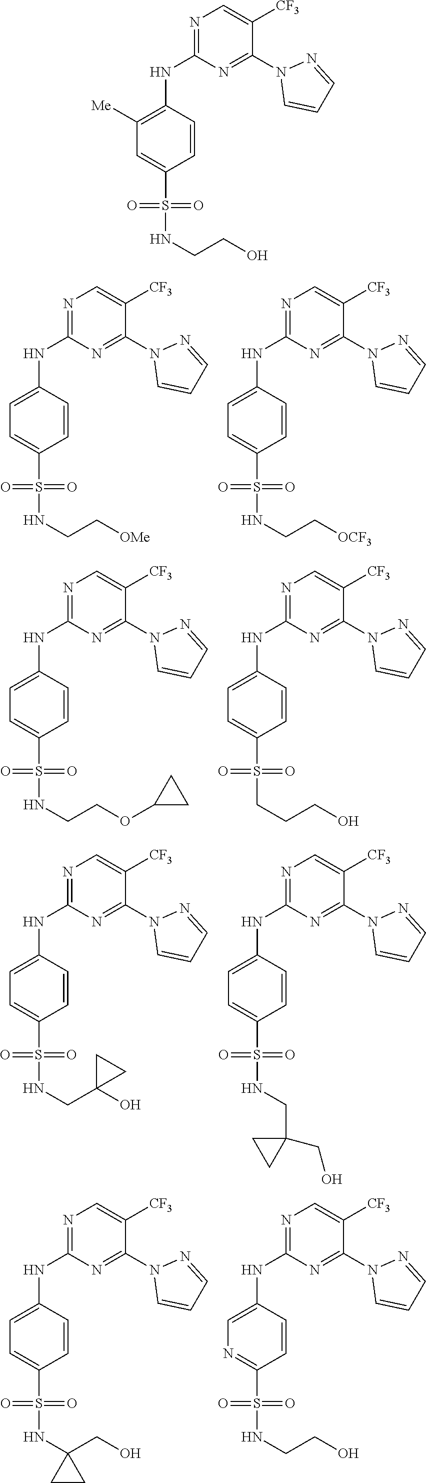

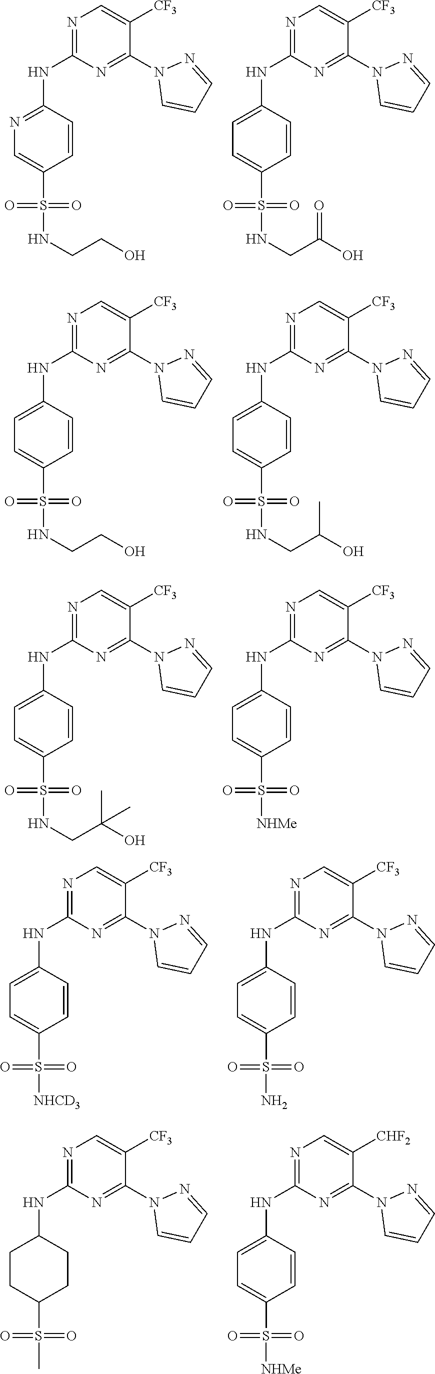

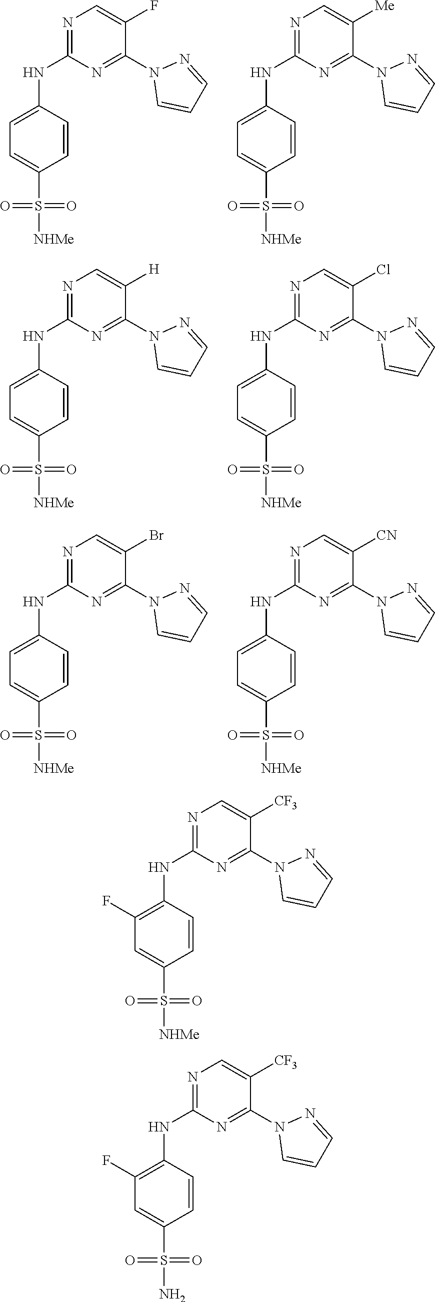

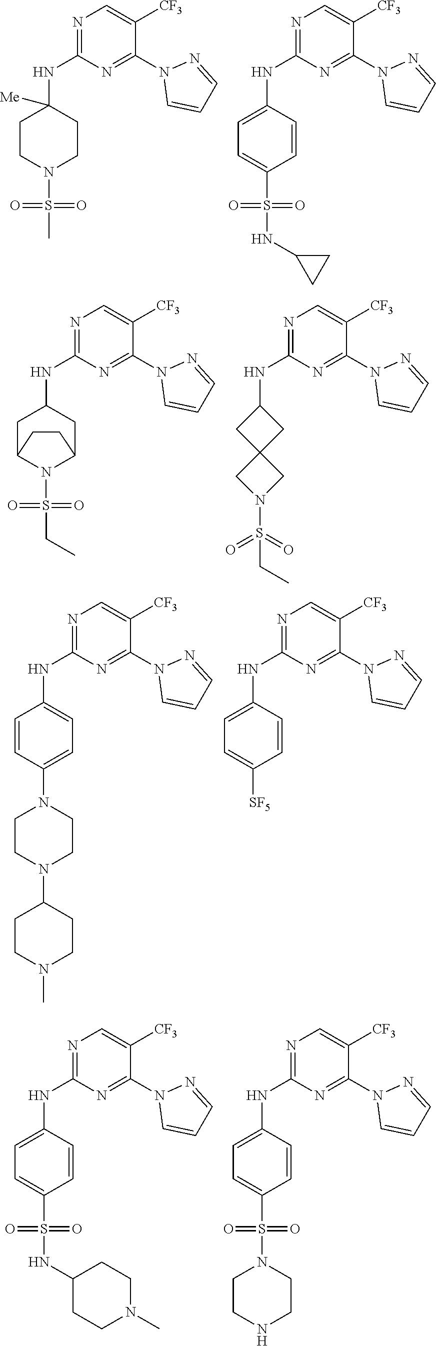

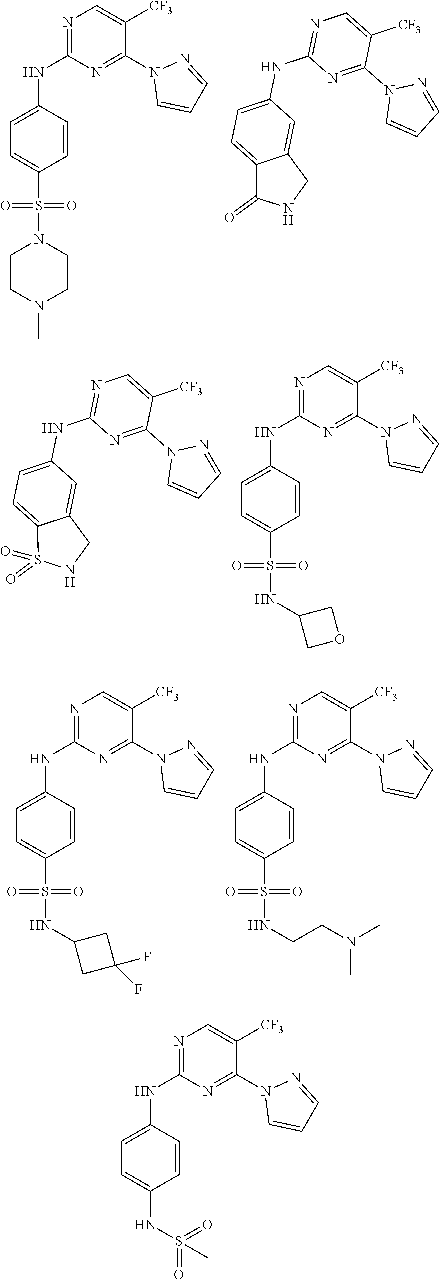

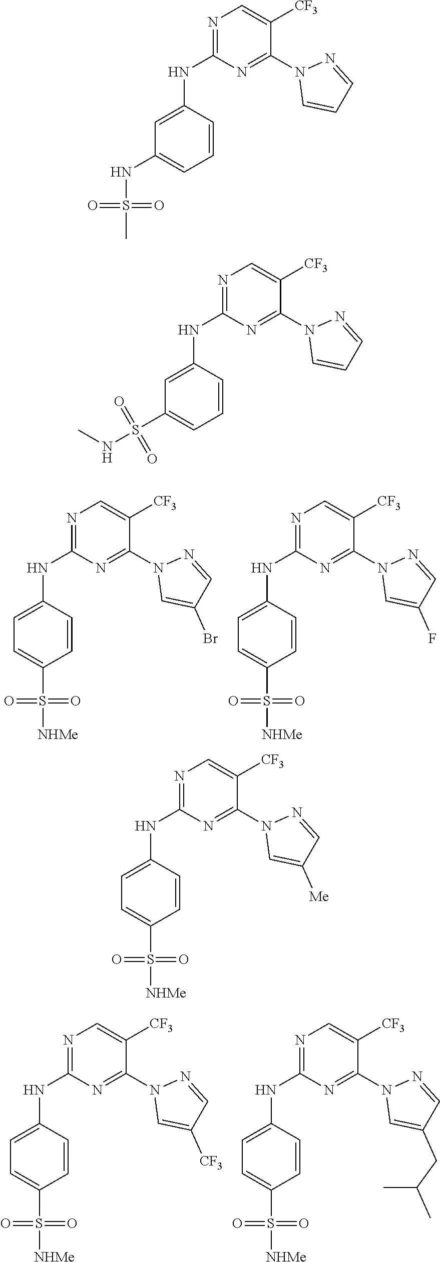

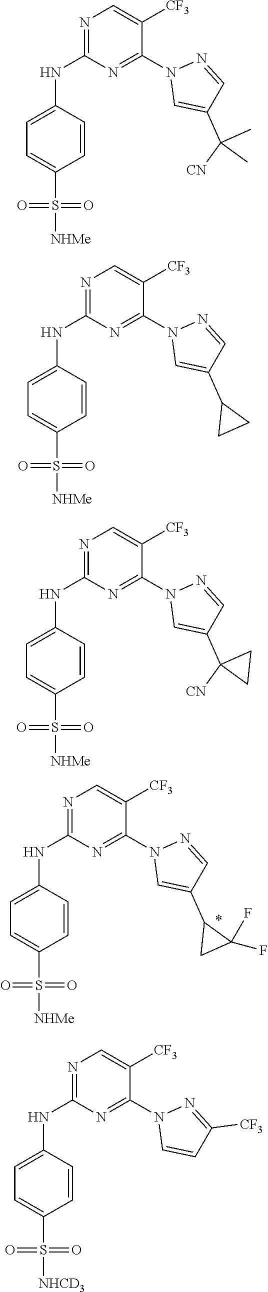

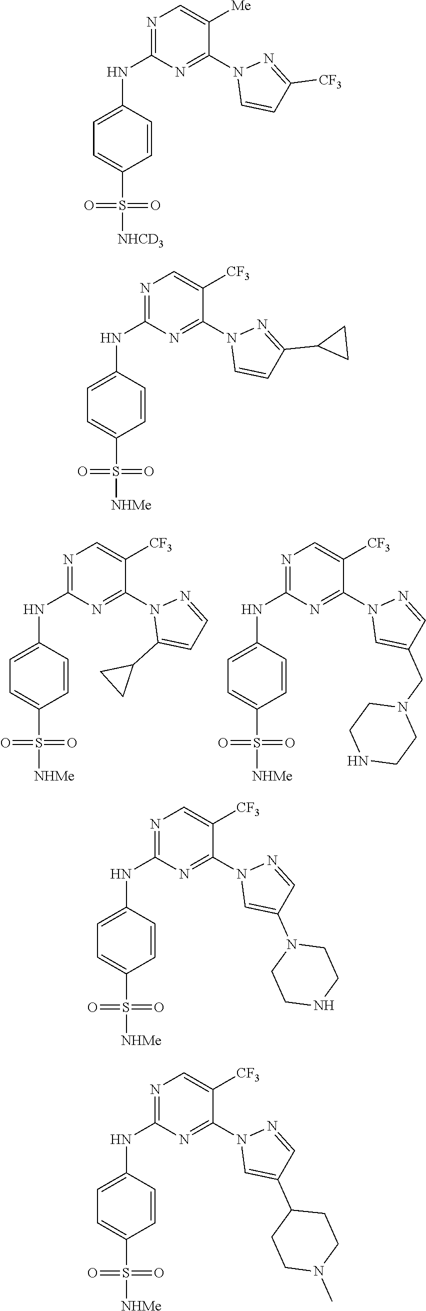

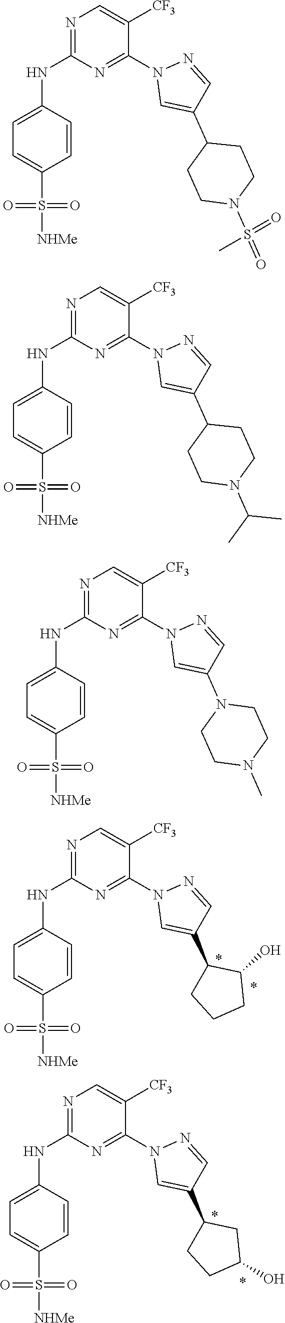 [SMILES: CNS(=O)(=O)c1ccc(Nc2ncc(C(F)(F)F)c(-n3cc(C4CCN(S(C)(=O)=O)CC4)cn3)n2)cc1 ; CNS(=O)(=O)c1ccc(Nc2ncc(C(F)(F)F)c(-n3cc(C4CCN(C(C)C)CC4)cn3)n2)cc1 ; CNS(=O)(=O)c1ccc(Nc2ncc(C(F)(F)F)c(-n3cc(N4CCN(C)CC4)cn3)n2)cc1 ; CNS(=O)(=O)c1ccc(Nc2ncc(C(F)(F)F)c(-n3cc([C@@H]4CCC[C@H]4O)cn3)n2)cc1 ; CNS(=O)(=O)c1ccc(Nc2ncc(C(F)(F)F)c(-n3cc([C@@H]4CC[C@@H](O)C4)cn3)n2)cc1]

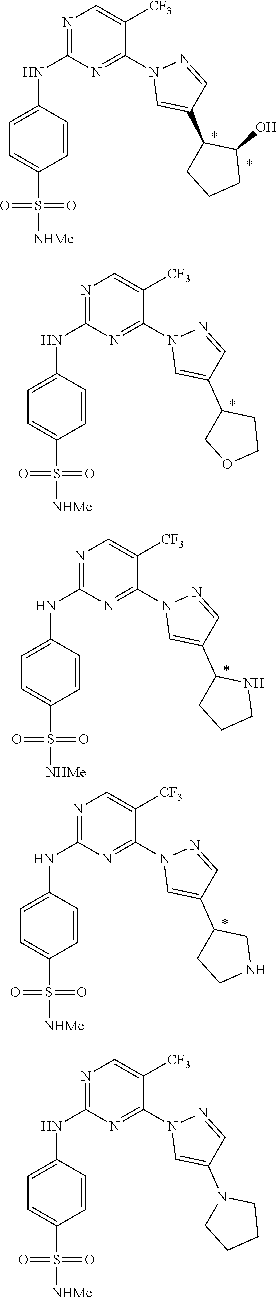

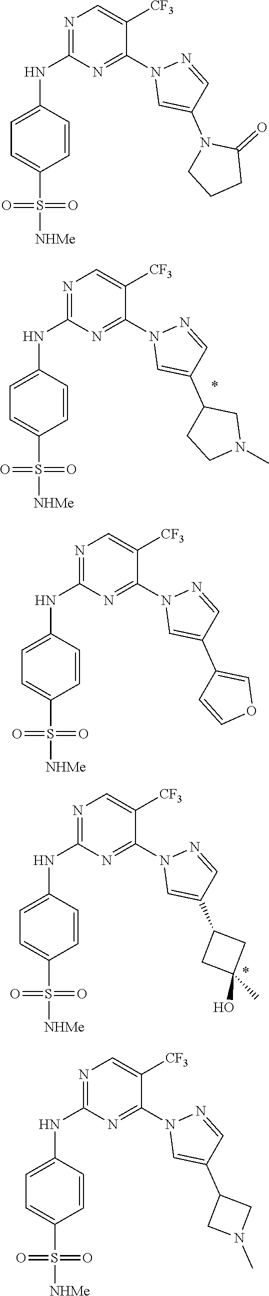 [SMILES: CNS(=O)(=O)c1ccc(Nc2ncc(C(F)(F)F)c(-n3cc(N4CCCC4=O)cn3)n2)cc1 ; CNS(=O)(=O)c1ccc(Nc2ncc(C(F)(F)F)c(-n3cc(C4CCN(C)C4)cn3)n2)cc1 ; CNS(=O)(=O)c1ccc(Nc2ncc(C(F)(F)F)c(-n3cc(-c4ccoc4)cn3)n2)cc1 ; CNS(=O)(=O)c1ccc(Nc2ncc(C(F)(F)F)c(-n3cc([C@H]4C[C@](C)(O)C4)cn3)n2)cc1 ; CNS(=O)(=O)c1ccc(Nc2ncc(C(F)(F)F)c(-n3cc(C4CN(C)C4)cn3)n2)cc1]

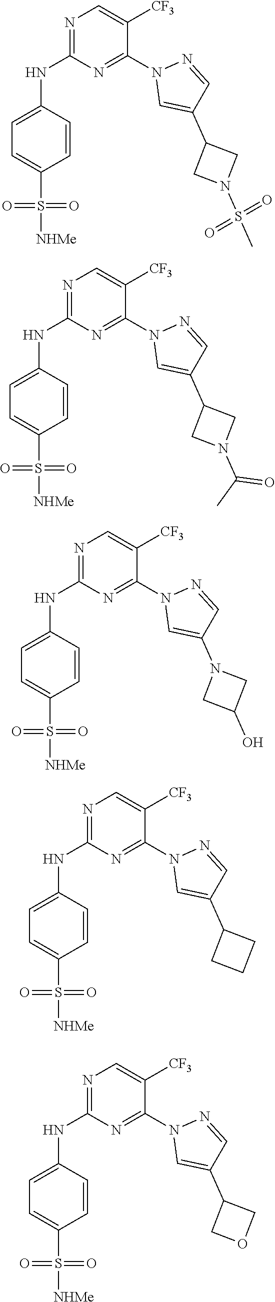 [SMILES: CNS(=O)(=O)c1ccc(Nc2ncc(C(F)(F)F)c(-n3cc(C4CN(S(C)(=O)=O)C4)cn3)n2)cc1 ; CNS(=O)(=O)c1ccc(Nc2ncc(C(F)(F)F)c(-n3cc(C4CN(C(C)=O)C4)cn3)n2)cc1 ; CNS(=O)(=O)c1ccc(Nc2ncc(C(F)(F)F)c(-n3cc(N4CC(O)C4)cn3)n2)cc1 ; CNS(=O)(=O)c1ccc(Nc2ncc(C(F)(F)F)c(-n3cc(C4CCC4)cn3)n2)cc1 ; CNS(=O)(=O)c1ccc(Nc2ncc(C(F)(F)F)c(-n3cc(C4COC4)cn3)n2)cc1]

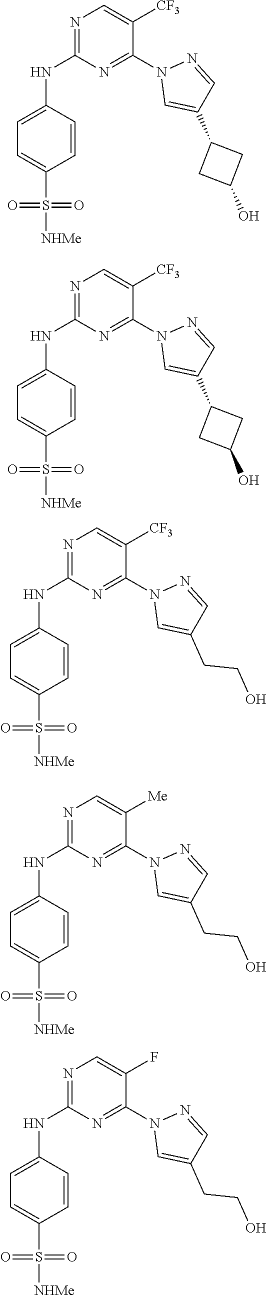 [SMILES: CNS(=O)(=O)c1ccc(Nc2ncc(C(F)(F)F)c(-n3cc([C@H]4C[C@@H](O)C4)cn3)n2)cc1 ; CNS(=O)(=O)c1ccc(Nc2ncc(C(F)(F)F)c(-n3cc([C@H]4C[C@H](O)C4)cn3)n2)cc1 ; CNS(=O)(=O)c1ccc(Nc2ncc(C(F)(F)F)c(-n3cc(CCO)cn3)n2)cc1 ; CNS(=O)(=O)c1ccc(Nc2ncc(C)c(-n3cc(CCO)cn3)n2)cc1 ; CNS(=O)(=O)c1ccc(Nc2ncc(F)c(-n3cc(CCO)cn3)n2)cc1]

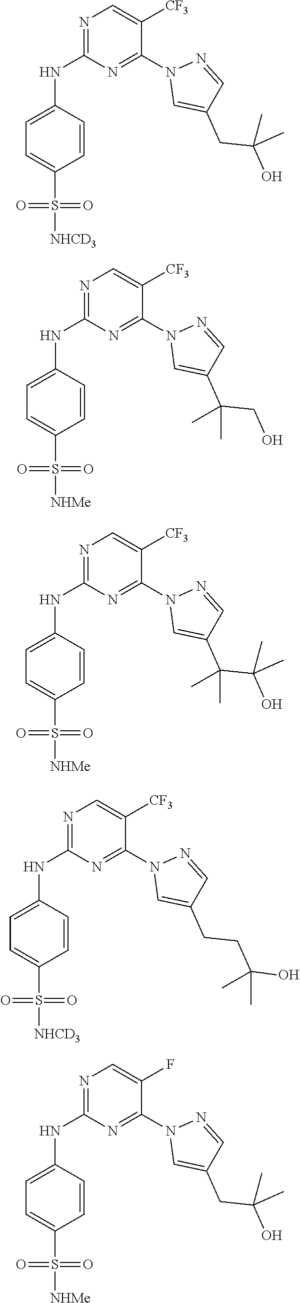

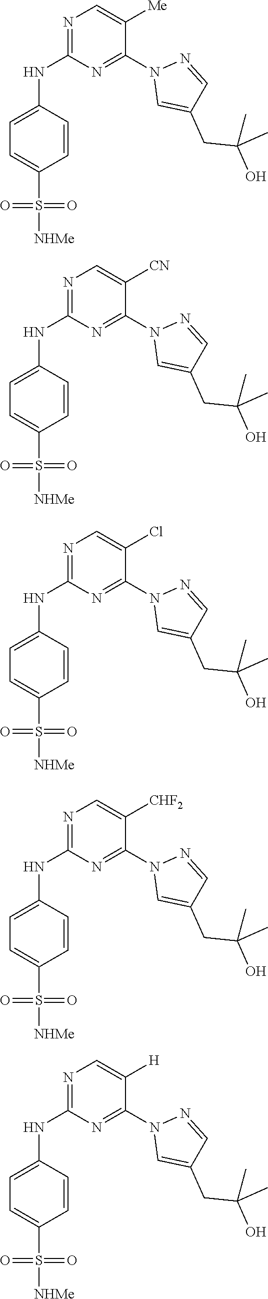 [SMILES: CNS(=O)(=O)c1ccc(Nc2ncc(C)c(-n3cc(CC(C)(C)O)cn3)n2)cc1 ; CNS(=O)(=O)c1ccc(Nc2ncc(C#N)c(-n3cc(CC(C)(C)O)cn3)n2)cc1 ; CNS(=O)(=O)c1ccc(Nc2ncc(Cl)c(-n3cc(CC(C)(C)O)cn3)n2)cc1 ; CNS(=O)(=O)c1ccc(Nc2ncc(C(F)F)c(-n3cc(CC(C)(C)O)cn3)n2)cc1 ; CNS(=O)(=O)c1ccc(Nc2nccc(-n3cc(CC(C)(C)O)cn3)n2)cc1]

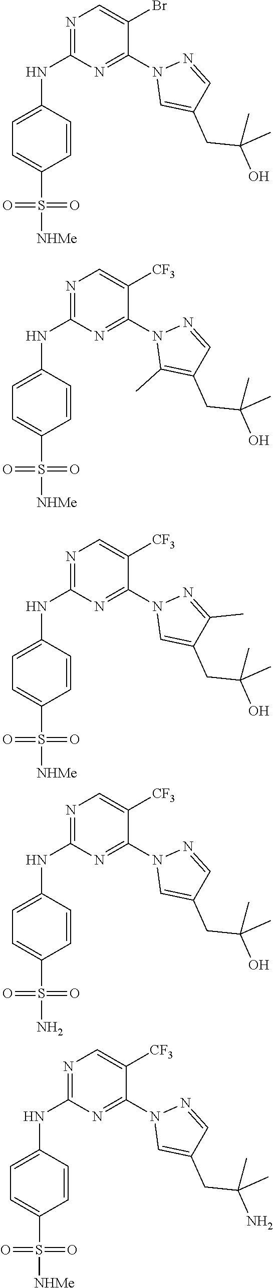 [SMILES: CNS(=O)(=O)c1ccc(Nc2ncc(Br)c(-n3cc(CC(C)(C)O)cn3)n2)cc1 ; CNS(=O)(=O)c1ccc(Nc2ncc(C(F)(F)F)c(-n3ncc(CC(C)(C)O)c3C)n2)cc1 ; CNS(=O)(=O)c1ccc(Nc2ncc(C(F)(F)F)c(-n3cc(CC(C)(C)O)c(C)n3)n2)cc1 ; CC(C)(O)Cc1cnn(-c2nc(Nc3ccc(S(N)(=O)=O)cc3)ncc2C(F)(F)F)c1 ; CNS(=O)(=O)c1ccc(Nc2ncc(C(F)(F)F)c(-n3cc(CC(C)(C)N)cn3)n2)cc1]

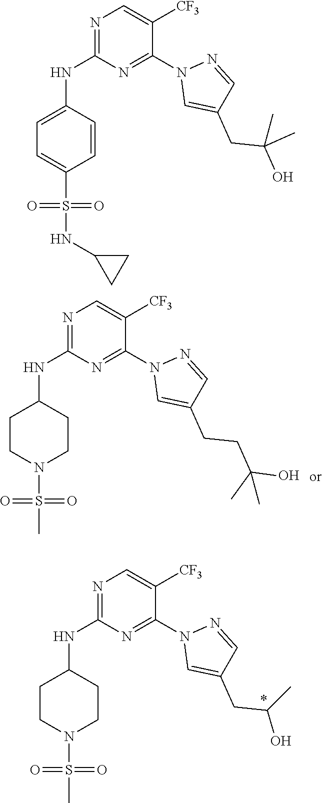

In some embodiments, the compounds of the present invention are selective cyclin-dependent kinase 2 (CDK2) inhibitors.

Another aspect of this invention provides pharmaceutical compositions, each including a therapeutically effective amount of a compound described herein, a pharmaceutically acceptable salt, tautomer, or stereoisomer thereof, in admixture with one or more physiologically or pharmaceutically acceptable carriers or excipients.

In some other embodiments, such pharmaceutical compositions may include a second therapeutic agent. Without limitation, examples of suitable second therapeutic agents include CDK4/6 inhibitors (e.g., Palbociclib, Abemaciclib, Ribociclib, or Trilaciclib), estrogen receptor antagonists, hormone therapy (e.g., letrazole, and fulvestrant), PARP inhibitors (e.g., Olaparib), S phase inhibitors (e.g., Carboplatin, Gemcitabine, Cisplatin, Topotecan), M phase inhibitors (e.g., Paclitaxel), and BCL-2 inhibitor (e.g., ABT-263).

Yet still another aspect of this invention provides a method for treating a subject suffering from a disease or disorder mediated by CDK2, comprising administering to the subject in need thereof an effective amount of the compound or the pharmaceutical composition described herein.

In some embodiments, the disease or disorder mediated by CDK2 is cancer or a disease caused by abnormal cell proliferation. Examples of cancer include, but not limited to, breast cancer, colorectal cancer, lung cancer, ovarian cancer, pancreatic cancer, melanoma, prostate cancer, glioblastoma, or sarcoma.

Yet still another aspect of this invention provides use of a compound described herein for the manufacture of a medicament for treating a disease or disorder mediated by CDK2.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications, and equivalents, which may be included within the spirit and scope of the invention as defined by the claims. Furthermore, in the detailed description of the present invention, numerous specific details are set forth to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

### Definitions

Unless the context indicates otherwise, references to Formula (I) in all sections of this document (including the uses, methods and other aspects of the invention) include references to all other sub-formula, sub-groups, preferences, embodiments and examples as defined herein.

Unless otherwise stated, the following terms used in the specification and claims have the meanings discussed below:

As used herein, the term “or” is meant to include both “and” and “or”. In other words, the term “or” may also be replaced with “and/or”.

In defining various terms, for example, “X” and “Z”, are used herein as generic symbols to represent various specific chemical elements.

As used herein, the term “unsaturated bond” refers to a double or triple bond.

As used herein, the term “unsaturated” or “partially unsaturated” refers to a moiety that includes at least one double or triple bond.

As used herein, the term “saturated” refers to a moiety that does not contain a double or triple bond, i.e., the moiety only contains single bonds.

As used herein, the term “alkyl” % by itself or as part of another substituent refers to a linear (i.e., unbranched or straight) or branched hydrocarbon chain radical consisting of carbon and hydrogen atoms, containing no unsaturation, having the stated number of carbon atoms (e.g., C₁-C₁₀ or C₁₋₁₀ alkyl). Whenever it appears herein, a numerical range such as “1 to 10” refers to each integer in the given range, e.g., “1 to 10 carbon atoms” means that the alkyl group can consist of 1 carbon atom, 2 carbon atoms, 3 carbon atoms, 4 carbon atoms, etc., up to and including 10 carbon atoms, although the present definition also covers the occurrence of the term “alkyl” where no numerical range is designated.% Representative saturated linear or straight alkyl includes, but not limited to, -methyl, -ethyl, -n-propyl, -n-butyl, -n-pentyl, and -n-hexyl; while saturated branched alkyl includes, but not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like. The alkyl is attached to the parent molecule by a single bond. Unless stated otherwise in the specification, an alkyl group is optionally substituted by one or more substituents.

When any H in “alkyl” is replaced with deuterium (D), the alkyl is a “deuterated alkyl”.

The term “alkylene” by itself or as part of another molecule means a divalent radical derived from an alkane, which can be a straight chain or branched chain. In this context, the prefixes (e.g., C₁₋₄, C₁₋₇, C₁₋₂₀, C₂₋₇, C₃₋₇, etc.) denote the number of carbon atoms, or range of number of carbon atoms. For example, the term “C₁₋₄alkylene,” as used herein, refers to an alkylene group having from 1 to 4 carbon atoms. Examples of linear C₁₋₈ alkylene groups include, but are not limited to, —(CH₂)ₙ— where n is an integer from 1 to 7, for example, —CH₂—, —CH₂CH₂CH₂—, and —CH₂CH₂CH₂CH₂—. Examples of branched C₁₋₇alkylene groups include, but are not limited to, —CH(CH₃)—, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

As used herein, the term “alkenyl” by itself or as part of another substituent refers to an unsaturated branched or straight-chain having at least one carbon-carbon double bond derived by the removal of one hydrogen atom from a single carbon atom of a parent alkene.

The group may be in either the cis or trans conformation about the double bond(s). Typical alkenyl groups include, but are not limited to, ethenyl, propenyl, and the like.

As used herein, the term “alkynyl” by itself or as part of another substituent refers to carbon chains which contain at least one carbon-carbon triple bond, and which may be linear or branched or combinations thereof. Examples of alkynyl include ethynyl, propargyl, 3-methyl-1-pentynyl, 2-heptynyl and the like.

As used herein, the term “cycloalkyl” by itself or as part of another substituent refers to a non-aromatic carbon-based ring composed of at least three carbon atoms. The term cycloalkyl includes monocyclic cycloalkyl, bicyclic cycloalkyl, polycyclic cycloalkyl, bridged cycloalkyl, fused cycloalkyl, and spiro cycloalkyl groups. In a bridged cycloalkyl, the rings share at least two common non-adjacent atoms. In a fused bicyclic cycloalkyl, two rings share a covalent bond. In a spirocyclic cycloalkyl group, one atom is common to two different rings.%

The term “cycloalkenyl” as used herein is a non-aromatic carbon-based ring composed of at least three carbon atoms and containing at least one carbon-carbon double bond, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like.

The term “heterocycloalkenyl” is a type of cycloalkenyl group as defined above, wherein at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted.

The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted.

The term “heterocycle” or “heterocyclyl” as used herein, refers to a group derived from a monocyclic, bridged bicyclic, fused bicyclic, spirocyclic or polycyclic moiety comprising at least one nonaromatic ring comprising one or more ring-forming heteroatoms independently selected from nitrogen, oxygen, and sulfur. The nitrogen atom may be substituted or unsubstituted (i.e., N or NR wherein R is H or another substituent, if defined).

The heterocyclyl can be saturated or partially unsaturated. In certain embodiments, a heterocyclyl may comprises 1 to 4 heteroatoms as ring members. The heterocyclyl groups of the present disclosure can be attached to the parent molecular moiety through a carbon atom or a heteroatom in the group. Thus, the term is inclusive of, but not limited to, “heterocycloalkyl”, “heteroaryl”, “bicyclic heterocycle” and “polycyclic heterocycle”.

As used herein, the term “halo” or “halogen” refers to fluorine (fluoro, —F), chlorine (chloro, —Cl), bromine (bromo, —Br), or iodine (iodo, -I).

The term “haloalkyl” refers to alkyl as defined above in which one or more of the hydrogen atoms have been replaced with a halogen independently selected from fluoro, chloro, bromo, and iodo. “Fluoroalkyl” means alkyl as defined above wherein one or more hydrogen atoms have been replaced by fluoro atoms. Unless otherwise specified with a number, a haloalkyl can include as many as chemically possible halo atoms as substituents on the alkyl group. For example, fluoroethyl can be —CH₂CF₃, —CHF—CH₃, or —CH₂CH₂F.

As used herein, the term “hydrogen” (or H) includes its isotopes of deuterium (D or ²H) and tritium (³H), meaning a or any hydrogen atom in the compounds of this invention can be replaced with either deuterium (D or ²H) and tritium (³H).

As used herein, the term “alkoxy” or “alkoxyl” refers to a saturated straight or branched hydrocarbon linked to an oxygen atom. Representative saturated straight chain alkoxys include methoxy, ethoxy, n-propoxy, n-butoxy, n-pentoxy, n-hextoxy, and the like; while saturated branched alkoxys include isopropoxyl, sec-butoxy, isobutoxy, tert-butoxy, isopentoxy, and the like. Cyclic alkoxy are referred to herein as a “cycloalkoxy”. “C₁₋₄alkoxy” refers to an alkyl with 1, 2, 3, or 4 carbon atoms. Alkoxy can be linked to a molecule by one or two attachment points.

The term “alkoxyalkyl”, as used herein, refers to an alkyl group substituted with one, two, or three alkoxy groups.

As used herein, the term “aryl” refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl. The “aryl” group can be substituted or unsubstituted.

As used herein, the term “heteroaryl” refers to a monocyclic or fused ring (i.e., rings which share an adjacent pair of atoms) of 5 to 12 ring atoms containing one, two, three or four ring heteroatoms selected from N, O or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, triazole, tetrazole, triazine, carbazole, benzimidazole, benzoxazole, benzthiazole, indazole and quinazoline. The heteroaryl group may be substituted or unsubstituted.

As used herein, the term “arylene” refers to a bidentate moiety obtained by removing two hydrogen atoms, one from each of two different aromatic ring atoms of an aromatic compound, which moiety has from 3 to 20 ring atoms (unless otherwise specified). Preferably, each ring has from 5 to 7 ring atoms.

As used herein, the term “hydroxyl” or “hydroxy” refers to the group —OH.

As used herein, the term “hydroxyalkyl” by itself or as part of another substituent refers to an alkyl group in which one or more of the hydrogen atoms are replaced with a hydroxyl substituent. Thus, the term “hydroxyalkyl” is meant to include monohydroxyalkyl, dihydroxyalkyl, trihydroxyalkyl, etc. In “hydroxyalkyl” group, the alkyl can be a linear (i.e., straight or unbranched) or branched alkyl, accordingly, the “hydroxyalkyl” group includes linear hydroxyalkyl and branched hydroxyalkyl.

As used herein, the term “cyano” or “—CN” refers to a group of —C≡N; and the term “cyanoalkyl” or “-alk-CN” refers to an alkyl group having at least one —CN substituent. In general, if a compound is attached to an “-alk-CN” group, the alkylene portion of the “-alk-CN” group is attached to the compound. In “cyanoalkyl” group, the alkyl can be a linear (i.e., straight or unbranched) or branched alkyl, accordingly, the “cyanoalkyl” group includes linear cyanoalkyl and branched cyanoalkyl.

As used herein, the term “-alk-” (alone or in combination with other terms) is an alkylene group, for example, -alk-N—R⁹R⁹.

As used herein, the term “oxo” (alone or in combination with another term) refers to ═O.

As used herein, the term “amino” or “amine” refers to —NH₂. The term “alkylamino” refers to a group of the formula —NHR, and “dialkylamino” refers to a group of the formula —NRR′, where each of R and R′ is independently an alkyl.

As used herein, the term “nitro,” refers to —NO₂.

As used herein, the term “SO₂” refers to sulfur dioxide, having a structure of

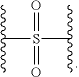 [SMILES: *S(*)(=O)=O]

As used herein, the term “—C(═O)—” or “—CO—” refers to a group of a structure of

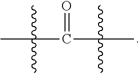 [SMILES: *C(*)=O]

As used herein, the term “carboxyalkyl” refers to an alkyl group substituted with one, two, or three carboxy groups.

The above-defined groups may include prefixes and/or suffixes that are commonly used in the art to create additional well-recognized substituent groups. As examples, the term “haloalkoxy” or “(haloalkyl)oxy” refers to a haloalkyl group attached to the parent molecular moiety through an oxygen atom. The term “(haloalkyl)oxyalkyl” refers to an alkyl group substituted with one, two, or three (haloalkyl)oxy groups.

As used herein, the term “absent” meaning that a defined variable is not present and replaced with a bond.

As sued herein, the term “bond” refers to a covalent linkage between two atoms, which may indicate a single bond, a double bond, or a triple bond.

As used herein, the term “stereoisomer” refers to isomers of identical constitution that differ only in spatial arrangement of atoms, rather than order of atomic connectivity. When a disclosed compound is named or depicted by structure without indicating stereochemistry, it is understood that the name or structure encompasses all possible stereoisomers, including essentially pure stereoisomers, as well as combination thereof. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

As used herein, the term “chiral” refers to the structural characteristic of a molecule that makes it impossible to superimpose it on its mirror image.

As used herein, the term “tautomer” refers to each of two or more isomers of a compound that exist together in equilibrium, and are readily interchanged by migration of an atom or group within the molecule. Thus, this disclosure is intended to cover all possible tautomer even when a structure depicts only one of them.

The term “optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with the alkyl group.

The term “pharmaceutically acceptable salts” refers to salts prepared from pharmaceutically acceptable non-toxic bases or acids including inorganic or organic bases and inorganic or organic acids, which are, within the scope of sound medical judgment, suitable for use in contact with the tissues of patients without excessive toxicity, irritation, allergic response, or other problem or complication commensurate with a reasonable benefit/risk ratio, and are effective for their intended use. Salts derived from inorganic bases include aluminum, ammonium, calcium, copper, ferric, ferrous, lithium, magnesium, manganic salts, manganous, potassium, sodium, zinc, and the like. Particularly preferred are ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethyl-aminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methyl-glucamine, morpholine, piperazine, piperidine, polyamine resins, procaine, purines, theobromine, triethylamine, trimethylamine, tripropylamine, tromethamine, and the like.

When a compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula (I) are meant to also include the pharmaceutically acceptable salts.

The terms a “pharmaceutical composition” refers to a mixture of one or more of the compounds described herein, or pharmaceutically acceptable salts or prodrugs thereof, with other chemical components, such as pharmaceutically acceptable excipients. The purpose of a pharmaceutical composition is to facilitate administration of a compound to an organism.

The term a “pharmaceutically acceptable excipient” refers to an inert substance added to a pharmaceutical composition to further facilitate administration of a compound. Examples of excipients include calcium carbonate, calcium phosphate, various sugars and types of starch, cellulose derivatives, gelatin, vegetable oils and polyethylene glycols.

As used herein, the term “therapeutically effective amount” refers to that amount of the compound being administered which will relieve to some extent one or more of the symptoms of the disorder being treated. In reference to the treatment of cancer, a therapeutically effective amount refers to that amount which has the effect of: (1) reducing the size of the tumor; (2) inhibiting tumor metastasis; (3) inhibiting tumor growth; and/or (4) relieving one or more symptoms associated with the cancer.

As used herein, the term “subject” or “patient” is used interchangeably and refers to any animal subject, including but not limited to human beings, laboratory animals (e.g., primates, rats, mice), livestock (e.g., cows, sheep, goats, pigs, turkeys, and chickens), and household pets (e.g., dogs, cats, and rodents).

The compounds taught herein can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration can be by continuous infusion over a selected period of time.

### Isomeric Forms

The present invention provides novel compounds of Formula (I), or a pharmaceutically acceptable salt therefore, as CDK2 inhibitors.

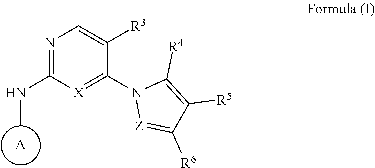

It will be appreciated that certain compounds of Formula I (or salts, prodrugs, or conjugates) may exist in, and be isolated in, isomeric forms, including tautomeric forms, geometric isomers (i.e., cis- or trans-isomers), optical isomers (i.e., enantiomers and diastereomers), racemic forms, or any mixture of the isomeric forms described above. It is to be understood that the present invention encompasses a compound of Formula I in any of the isomeric forms or as a mixture thereof, for example, in the form of an active single enantiomer, racemic, or any mixture thereof. The present invention is meant to comprehend all such isomeric forms of the compounds of Formula (I).

In addition, a compound of Formula (I) (or salt, prodrug or conjugate thereof) may exhibit polymorphism or may form a solvate with water or an organic solvent. The present invention also encompasses any such polymorphic form, any solvate or any mixture thereof.

The following examples are illustrative of select embodiments of the present invention and are not meant to limit the scope of the invention.

### Example 1: 2-(1-(5-fluoro-2-((1-(methylsulfonyl)piperidin-4-yl)amino)pyrimidin-4-yl)-1H-pyrazol-4-yl)propan-2-ol

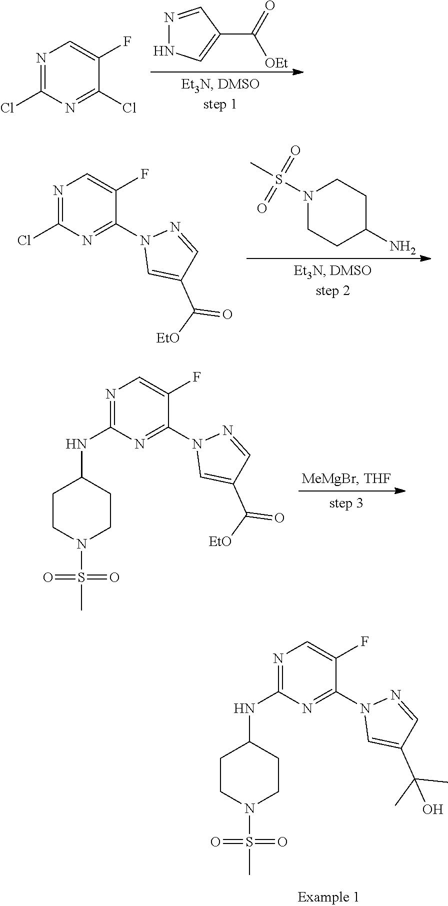

### Step 1: ethyl 1-(2-chloro-5-fluoropyrimidin-4-yl)-1H-pyrazole-4-carboxylate

To a solution of 2,4-dichloro-5-fluoropyrimidine (100.0 mg, 0.6 mmol) in DMSO (2 mL) was added ethyl 1H-pyrazole-4-carboxylate (125.9 mg, 0.9 mmol) and Et₃N (0.25 mL, 1.8 mmol). The reaction was stirred at 50° C. for 40 min. The mixture was extracted with ethyl acetate and concentrated in vacuo. The residue was purified by flash chromatography (eluting with 0%-50% ethyl acetate in petroleum ether) to afford the ethyl 1-(2-chloro-5-fluoropyrimidin-4-yl)-1H-pyrazole-4-carboxylate as a white solid.

MS (ES-API positive): 271.1 (M+H)⁺.

### Step 2: ethyl 1-(5-fluoro-2-((1-(methylsulfonyl)piperidin-4-yl)amino)pyrimidin-4-yl)-1H-pyrazole-4-carboxylate

To a solution of ethyl 1-(2-chloro-5-fluoropyrimidin-4-yl)pyrazole-4-carboxylate (70.0 mg, 0.3 mmol) in DMSO (1.5 mL) was added 1-(methylsulfonyl)piperidin-4-amine (69.2 mg, 0.4 mmol) and Et₃N (0.1 mL, 0.8 mmol). The reaction was stirred at 50° C. for overnight. The mixture was diluted with ethyl acetate and H₂O. The water layer was extracted with ethyl acetate and concentrated in vacuo. The residue was purified by flash chromatography (eluting with 0%-50% ethyl acetate in petroleum ether) to afford the ethyl 1-(5-fluoro-2-((1-(methylsulfonyl)piperidin-4-yl)amino)pyrimidin-4-yl)-1H-pyrazole-4-carboxylate as a white solid.

MS (ES-API positive): 413.3 (M+H)⁺.

### Step 3: 2-(1-(5-fluoro-2-((1-(methylsulfonyl)piperidin-4-yl)amino)pyrimidin-4-yl)-1H-pyrazol-4-yl)propan-2-ol

To a solution of ethyl 1-(5-fluoro-2-((1-(methylsulfonyl)piperidin-4-yl)amino)pyrimidin-4-yl)-1H-pyrazole-4-carboxylate (54.0 mg, 0.1 mmol) in THF (1.0 mL) was added bromo(methyl)magnesium (1 M in THF, 0.4 mL) at 0° C. The reaction was stirred at 0° C. for 30 min and rt for 4.5 h. The mixture was diluted with ethyl acetate and water, extracted with ethyl acetate and concentrated in vacuo. The residue was purified by flash chromatography (eluting with 0% to 50% ethyl acetate in petroleum ether) to afford the 2-(1-(5-fluoro-2-((1-(methylsulfonyl)piperidin-4-yl)amino)pyrimidin-4-yl)-1H-pyrazol-4-yl)propan-2-ol as a yellow liquid.

MS (ES-API positive): 399.3 (M+H)⁺.

¹H NMR (400 MHz, CD₃OD) δ 8.38 (s, 1H), 8.35 (d, J=4.3 Hz, 1H), 7.86 (s, 1H), 3.99-3.93 (m, 1H), 3.71 (d, J=12.3 Hz, 2H), 2.97 (td, J=12.0, 2.1 Hz, 2H), 2.86 (s, 3H), 2.12 (dd, J=12.8, 3.0 Hz, 2H), 1.65 (ddd, J=12.7, 11.2, 4.0 Hz, 2H), 1.57 (s, 6H).

### Example 2: 4-((4-(4-(2-hydroxy-2-methylpropyl)-1H-pyrazol-1-yl)-5-(trifluoromethyl)pyrimidin-2-yl)amino)-N-methylbenzenesulfonamide

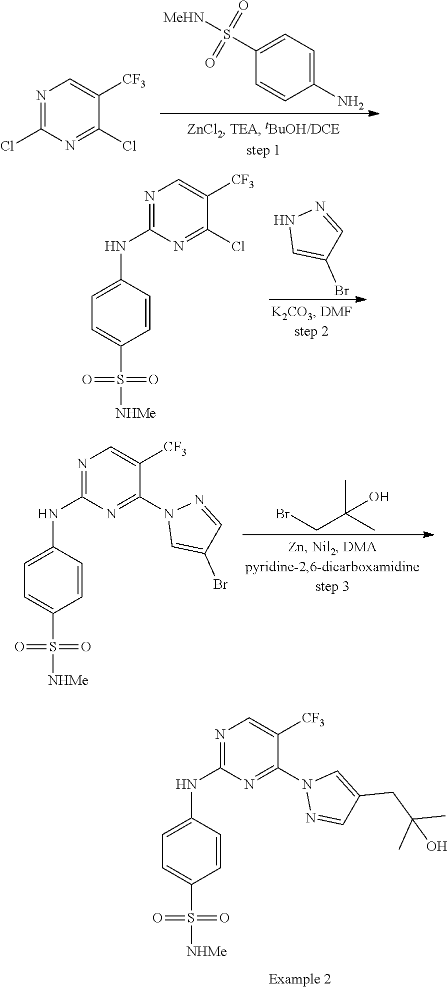

### Step 1: 4-((4-chloro-5-(trifluoromethyl)pyrimidin-2-yl)amino)-N-methylbenzenesulfonamide

To a solution of 2,4-dichloro-5-(trifluoromethyl)pyrimidine (3.5 g, 16.1 mmol) in 2-methylpropan-2-ol (60 mL) and 1,2-dichloroethane (60 mL) at 0° C. was added ZnCl₂ (2 M in THF, 8.9 mL) cautiously over 20 min and stirred at 0° C. for 30 min. A solution of 4-amino-N-methyl-benzenesulfonamide (3.0 g, 16.1 mmol) in 2-methylpropan-2-ol (30 mL) and 1,2-dichloroethane (30 mL) was added, followed by TEA (2.5 mL, 17.7 mmol) in 2-methylpropan-2-ol (30 mL) and 1,2-dichloroethane (30 mL) at 0° C. The reaction was stirred at 0-20° C. for 4 h. Solvents were removed in vacuo. The residue was suspended in water (20 mL), sonicated for 30 min and filtered. 4-[[4-Chloro-5-(trifluoromethyl)pyrimidin-2-yl]amino]-N-methyl-benzenesulfonamide was obtained as a yellow solid without further purification.

MS (ES-API positive): 367.1 (M+H)⁺

### Step 2: 4-((4-(4-bromo-1H-pyrazol-1-yl)-5-(trifluoromethyl)pyrimidin-2-yl)amino)-N-methylbenzenesulfonamide

To a mixture of 4-[[4-chloro-5-(trifluoromethyl)pyrimidin-2-yl]amino]-N-methyl-benzenesulfonamide (0.9 g, 2.5 mmol) and K₂CO₃ (678.3 mg, 4.9 mmol) in DMF (10 mL) was added 4-bromo-1H-pyrazole (432.8 mg, 2.9 mmol) at 15° C. The reaction was stirred at 15° C. for 16 h. The reaction mixture was poured into H₂O (30 mL), extracted with ethyl acetate (25 mL×3). The organic phases were washed with brine, dried over Na₂SO₄, filtered and concentrated. The residue was stirred in ethyl acetate and petroleum ether (1/10, V/V, 20 mL) and then filtered. 4-[[4-(4-bromopyrazol-1-yl)-5-(trifluoromethyl)pyrimidin-2-yl]amino]-N-methyl-benzenesulfonamide was obtained as an off-white solid.

MS (ES-API positive): 476.9, 478.9 (M+H)⁺.

### Step 3: 4-((4-(4-(2-hydroxy-2-methylpropyl)-1H-pyrazol-1-yl)-5-(trifluoromethyl)pyrimidin-2-yl)amino)-N-methylbenzenesulfonamide

To a solution of 4-[[4-(4-bromopyrazol-1-yl)-5-(trifluoromethyl)pyrimidin-2-yl]amino]-N-methyl-benzenesulfonamide (100.0 mg, 209.5 μmol) and 1-bromo-2-methyl-propan-2-ol (64.1 mg, 419.1 μmol) in DMA (1 mL) was added pyridine-2,6-dicarboxamidine (6.8 mg, 41.9 μmol), diiodonickel (13.1 mg, 41.9 μmol), Zn (54.8 mg, 838.1 μmol) and TBAI (116.1 mg, 314.3 μmol). The mixture was degassed and purged with N₂ for three times and stirred at 80° C. for 16 h. The reaction mixture was filtered. The filtrate was poured into water (5 mL), extracted with ethyl acetate (5 mL×3). The combined organic phases were dried over Na₂SO₄, filtered and concentrated in vacuo. The residue was purified by prep-HPLC to afford 4-[[4-[4-(2-hydroxy-2-methyl-propyl)pyrazol-1-yl]-5-(trifluoromethyl)pyrimidin-2-yl]amino]-N-methyl-benzenesulfonamide as a brown solid.

MS (ES-API positive): 471.0 (M+H)⁺.

¹H NMR (400 MHz, CD₃OD) δ 8.87 (s, 1H), 8.42 (s, 1H), 7.97 (d, J=8.8 Hz, 2H), 7.82 (d, J=8.8 Hz, 2H), 7.74 (s, 1H), 2.71 (s, 2H), 2.54 (s, 3H), 1.24 (s, 6H).

### Example 11: 4-((4-(4-cyclopentyl-1H-pyrazol-1-yl)-5-(trifluoromethyl)pyrimidin-2-yl)amino)-N-methylbenzenesulfonamide

### Example 12: 4-((4-(4-(cyclopent-1-en-1-yl)-1H-pyrazol-1-yl)-5-(trifluoromethyl)pyrimidin-2-yl)amino)-N-methylbenzenesulfonamide

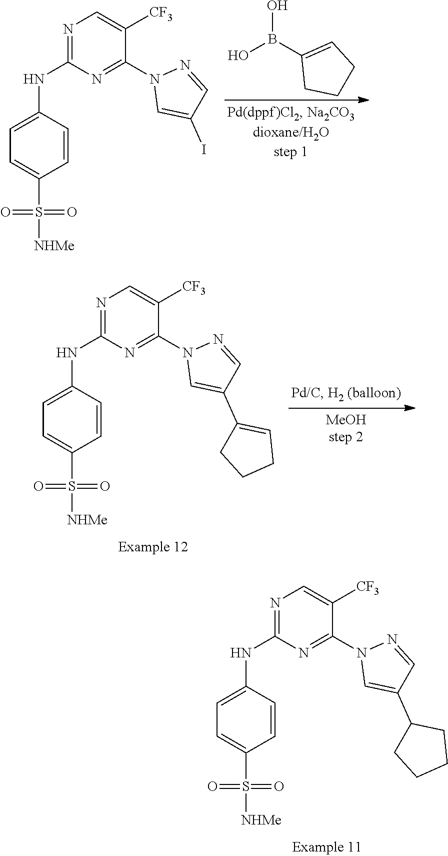 [SMILES: CNS(=O)(=O)c1ccc(Nc2ncc(C(F)(F)F)c(-n3cc(I)cn3)n2)cc1 ; OB(O)C1=CCCC1 ; CNS(=O)(=O)c1ccc(Nc2ncc(C(F)(F)F)c(-n3cc(C4=CCCC4)cn3)n2)cc1 ; CNS(=O)(=O)c1ccc(Nc2ncc(C(F)(F)F)c(-n3cc(C4CCCC4)cn3)n2)cc1]

### Step 1: 4-((4-(4-(cyclopent-1-en-1-yl)-1H-pyrazol-1-yl)-5-(trifluoromethyl)pyrimidin-2-yl)amino)-N-methylbenzenesulfonamide

To a solution of 4-[[4-(4-iodopyrazol-1-yl)-5-(trifluoromethyl)pyrimidin-2-yl]amino]-N-methyl-benzenesulfonamide (450.0 mg, 858.4 μmol) and cyclopenten-1-ylboronic acid (144.1 mg, 1.3 mmol) in H₂O (2 mL) and dioxane (4 mL) was added Pd(dppf)Cl₂ (125.6 mg, 171.7 μmol) and Na₂CO₃ (181.9 mg, 1.7 mmol). The reaction was stirred at 90° C. for 2 h. The mixture was poured into water (10 mL) and extracted with ethyl acetate (10 mL×3). The combined organic phases were washed with brine, dried over Na₂SO₄ and filtered and concentrated in vacuo. The residue was purified by flash chromatography (eluting with 0%-50% ethyl acetate in petroleum ether) to provide compound 4-[[4-[4-(cyclopenten-1-yl)pyrazol-1-yl]-5-(trifluoromethyl)pyrimidin-2-yl]amino]-N-methyl-benzenesulfonamide as a brown solid.

MS (ES-API positive): 465.2 (M+H)⁺.

¹H NMR (500 MHz, CD₃OD) δ 8.87 (s, 1H), 8.44 (s, 1H), 8.00 (s, 1H), 7.97 (d, J=8.9 Hz, 2H), 7.84-7.80 (m, 2H), 6.14 (t, J=2.1 Hz, 1H), 2.69-2.65 (m, 2H), 2.55 (d, J=2.6 Hz, 1H), 2.54 (s, 3H), 2.53-2.49 (m, 1H), 2.08-2.01 (m, 2H).

### Step 2: 4-((4-(4-cyclopentyl-1H-pyrazol-1-yl)-5-(trifluoromethyl)pyrimidin-2-yl)amino)-N-methylbenzenesulfonamide

To a solution of 4-[[4-[4-(cyclopenten-1-yl)pyrazol-1-yl]-5-(trifluoromethyl)pyrimidin-2-yl]amino]-N-methyl-benzenesulfonamide (50.0 mg, 107.6 μmol) in THF (5 mL) and ethyl acetate (5 mL) was added Pd/C (22.9 mg, 21.5 μmol, 10% purity). The reaction was stirred under 20 psi H₂ at 20° C. for 2 h. The mixture was filtered and concentrated. The residue was purified by prep-HPLC (eluting with 70%-90% acetonitrile in water) to provide compound 4-[[4-(4-cyclopentylpyrazol-1-yl)-5-(trifluoromethyl)pyrimidin-2-yl]amino]-N-methyl-benzenesulfonamide as a white solid.

MS (ES-API positive): 467.1 (M+H)⁺.

¹H NMR (400 MHz, CD₃OD) δ 8.86 (s, 1H), 8.34 (s, 1H), 7.96 (d, J=8.8 Hz, 2H), 7.81 (d, J=8.8 Hz, 2H), 7.74 (s, 1H), 3.04 (t, J=8.2 Hz, 1H), 2.54 (s, 3H), 2.18-2.02 (m, 2H), 1.92-1.66 (m, 4H), 1.66-1.53 (m, 2H).

### Example 16: N-methyl-4-[[4-pyrazol-1-yl-5-(trifluoromethyl) pyrimidin-2-yl]amino]benzene sulfonamide

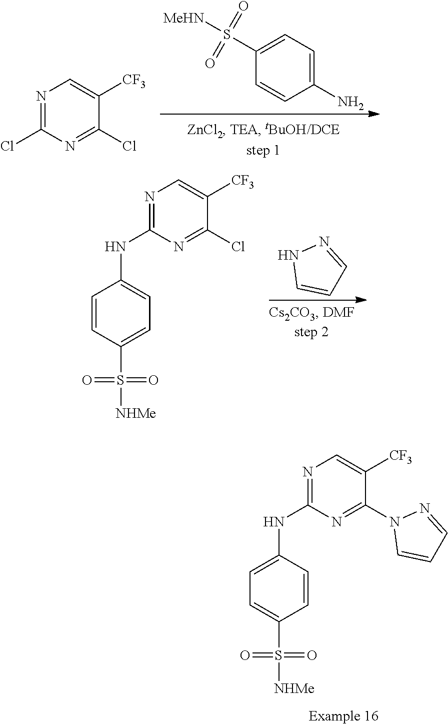 [SMILES: FC(F)(F)c1cnc(Cl)nc1Cl ; CNS(=O)(=O)c1ccc(N)cc1 ; CNS(=O)(=O)c1ccc(Nc2ncc(C(F)(F)F)c(Cl)n2)cc1 ; c1cn[nH]c1 ; CNS(=O)(=O)c1ccc(Nc2ncc(C(F)(F)F)c(-n3cccn3)n2)cc1]

### Step 1: 4-[[4-chloro-5-(trifluoromethyl) pyrimidin-2-yl]amino]-N-methyl-benzene sulfonamide

Same as Example 2.

### Step 2: N-methyl-4-[[4-pyrazol-1-yl-5-(trifluoromethyl) pyrimidin-2-yl]amino]benzene sulfonamide

To a solution of 4-[[4-chloro-5-(trifluoromethyl) pyrimidin-2-yl]amino]-N-methyl-benzene sulfonamide (20.0 g, 54.5 mmol) and 1H-pyrazole (4.5 g, 65.4 mmol) in DMSO (100 mL) was added Cs₂CO₃ (35.5 g, 109.0 mmol). The reaction was stirred at 80° C. for 2 h. The reaction mixture was poured into water (400 mL) and filtered. The solid was purified by prep-HPLC (eluting with 40%-70% acetonitrile in water). Compound N-methyl-4-[[4-pyrazol-1-yl-5-(trifluoromethyl) pyrimidin-2-yl]amino]benzene sulfonamide was obtained as a yellow solid.

MS (ES-API positive): 399.0 (M+H)⁺.

¹H NMR (400 MHz, CD₃OD) δ 8.89 (s, 1H), 8.57 (d, J=2.7 Hz, 1H), 7.96 (d, J=8.7 Hz, 2H), 7.88-7.76 (m, 3H), 6.61 (dd, J=1.6, 2.6 Hz, 1H), 2.54 (s, 3H).

### Example 32: 2-(ethyldioxo-λ6-sulfanyl)-6-{[4-(pyrazol-1-yl)-5-(trifluoromethyl) pyrimidin-2-yl]amino}-2-azaspiro[3.3]heptane

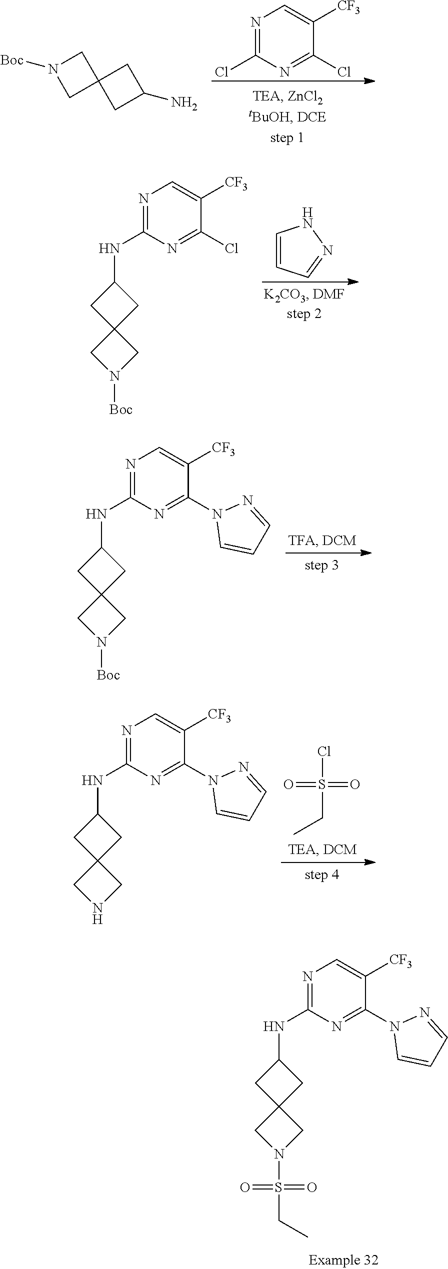

### Example 32

### Step 1: 2-methylpropan-2-yl 6-{[4-chloro-5-(trifluoromethyl) pyrimidin-2-yl]amino}-2-azaspiro[3.3]heptane-2-carboxylate

To a mixture of 2,4-dichloro-5-(trifluoromethyl) pyrimidine (766.5 mg, 3.5 mmol) in ^{t}BuOH:DCE=1:1 (10 mL) was added ZnCl₂ (5.2 mL, 5.2 mmol) at 0° C. for 0.5 h. A mixture of 2-methylpropan-2-yl 6-amino-2-azaspiro[3.3]heptane-2-carboxylate (500.0 mg, 2.4 mmol) in ^{t}BuOH:DCE=1:1 (10 mL) was added, followed by TEA (0.4 mL, 2.6 mmol) at 0° C. The reaction was stirred at rt for 16 h and concentrated in vacuo. The residue was diluted with H₂O and extracted with ethyl acetate. The combined organic layers were washed with brine, dried over Na₂SO₄, and concentrated. The residue was purified by flash chromatography (eluting with 23%-30% ethyl acetate in petroleum ether) to afford 2-methylpropan-2-yl 6-{[4-chloro-5-(trifluoromethyl) pyrimidin-2-yl]amino}-2-azaspiro[3.3]heptane-2-carboxylate as a yellow solid.

MS (ES-API positive): 393.1 (M+H)⁺.

### Step 2: 2-methylpropan-2-yl 6-{[4-(pyrazol-1-yl)-5-(trifluoromethyl) pyrimidin-2-yl]amino}-2-azaspiro[3.3]heptane-2-carboxylate

To a mixture of 2-methylpropan-2-yl 6-{[4-chloro-5-(trifluoromethyl) pyrimidin-2-yl]amino}-2-azaspiro[3.3]heptane-2-carboxylate (630.0 mg, 1.6 mmol) in DMF (7 mL) was added 1H-pyrazole (218.4 mg, 3.2 mmol) and K₂CO₃ (665.0 mg, 4.8 mmol). The reaction mixture was stirred at 80° C. for 16 h and concentrated in vacuo. The residue was diluted with H₂O and extracted with ethyl acetate. The combined organic layers were washed with brine, dried over Na₂SO₄, and concentrated. The residue was purified by flash chromatography (eluting with 50%-55% ethyl acetate in petroleum ether) to afford 2-methylpropan-2-yl 6-{[4-(pyrazol-1-yl)-5-(trifluoromethyl)pyrimidin-2-yl]amino}-2-azaspiro[3.3]heptane-2-carboxylate as a white solid.

MS (ES-API positive): 425.2 (M+H)⁺.

### Step 3: 6-{[4-(pyrazol-1-yl)-5-(trifluoromethyl) pyrimidin-2-yl]amino}-2-azaspiro[3.3]heptane

To a mixture of 2-methylpropan-2-yl 6-{[4-(pyrazol-1-yl)-5-(trifluoromethyl) pyrimidin-2-yl]amino}-2-azaspiro[3.3]heptane-2-carboxylate (50.0 mg, 0.1 mmol) in DCM (0.5 mL) was added TFA (0.5 mL, 6.5 mmol). The reaction mixture was stirred at 35° C. for 8 h and concentrated in vacuo. The residue was directly used for next step without further purification.

MS (ES-API positive): 325.2 (M+H)⁺.

### Step 4: 2-(ethyl dioxo-λ6-sulfanyl)-6-{[4-(pyrazol-1-yl)-5-(trifluoromethyl) pyrimidin-2-yl]amino}-2-azaspiro[3.3]heptane

To a mixture of 6-{[4-(pyrazol-1-yl)-5-(trifluoromethyl)pyrimidin-2-yl]amino}-2-azaspiro[3.3]heptane (38.2 mg, 0.1 mmol) in DCM (0.5 mL) was added TEA (0.3 mL, 2.1 mmol) and stirred for 15 min. Ethane sulfonyl chloride (15.1 mg, 0.1 mmol) was added and stirred for another 2 h. The mixture was diluted with H₂O and extracted with DCM. The combined organic layers were washed with brine, dried over Na₂SO₄, and concentrated in vacuo. The residue was purified by flash chromatography (eluting with 55%-60% ethyl acetate in petroleum ether) to afford 2-(ethyl dioxo-λ6-sulfanyl)-6-{[4-(pyrazol-1-yl)-5-(trifluoromethyl) pyrimidin-2-yl]amino}-2-azaspiro[3.3]heptane as a white solid.

MS (ES-API positive): 417.2 (M+H)⁺.

¹H NMR (400 MHz, DMSO-d₆) δ 8.76-8.36 (m, 3H), 7.90 (d, J=5.6 Hz, 1H), 6.65-6.57 (m, 1H), 4.37-4.24 (m, 1H), 3.95 (d, J=10.2 Hz, 2H), 3.82 (s, 2H), 3.07 (t, J=7.3 Hz, 2H), 2.60 (s, 2H), 2.23 (dd, J=14.7, 6.4 Hz, 2H), 1.20 (t, J=7.4 Hz, 3H).

### Example 36: 4-((4-(1H-pyrazol-1-yl)-5-(trifluoromethyl) pyrimidin-2-yl) amino)-N-(2-hydroxyethyl) benzene sulfonamide

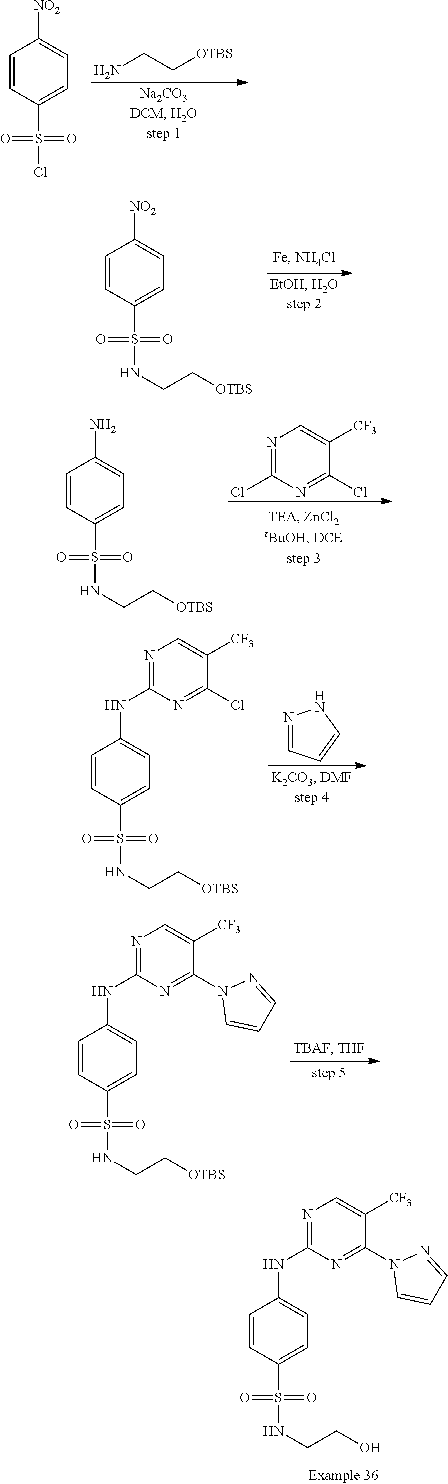

### Step 1: N-(2-((tert-butyldimethylsilyl) oxy) ethyl)-4-nitrobenzenesulfonamide

To a mixture of 2-((tert-butyldimethylsilyl) oxy) ethan-1-amine (9.4 g, 53.8 mmol) and Na₂CO₃ (14.9 g, 140.4 mmol) in H₂O (150 mL) and DCM (150 mL) was added 4-nitrobenzenesulfonyl chloride (10.4 g, 46.8 mmol) at 0° C. The reaction was stirred at 30° C. for 2 h. The mixture was diluted with DCM and saturated NaHCO₃ solution. The organic layer was separated, washed with brine, concentrated in vacuo to afford the crude compound N-(2-((tert-butyldimethylsilyl) oxy) ethyl)-4-nitrobenzenesulfonamide as a white solid without further purification.

MS (ES-API positive): 361.1 (M+H)⁺.

### Step 2: 4-amino-N-(2-((tert-butyldimethylsilyl) oxy) ethyl) benzene sulfonamide

To a solution of N-(2-((tert-butyldimethylsilyl) oxy) ethyl)-4-nitrobenzenesulfonamide in EtOH (150 mL) and H₂O (100 mL) was added Fe (20.9 g, 374.4 mmol) and NH₄Cl (37.6 g, 701.9 mmol). The reaction was stirred at 65° C. for 1 h. The mixture was diluted with ethyl acetate, filtered and concentrated in vacuo. The residue was diluted with ethyl acetate, washed with saturated NaHCO₃, dried and concentrated. The residue was purified by flash chromatography (eluting with 3%-16% methanol in dichloromethane) to afford the compound 4-amino-N-(2-((tert-butyldimethylsilyl) oxy) ethyl) benzene sulfonamide as a white solid.

MS (ES-API positive): 331.2 (M+H)⁺.

### Step 3: N-(2-((tert-butyldimethylsilyl) oxy) ethyl)-4-((4-chloro-5-(trifluoromethyl) pyrimidin-2-yl) amino) benzene sulfonamide

To a solution of 2,4-dichloro-5-(trifluoromethyl) pyrimidine (14.5 g, 66.7 mmol) in ^{t}BuOH (110 mL) and DCE (110 mL) was added ZnCl₂ (73.3 mL, 73.3 mmol) at 0° C. and stirred at 0° C. for 1 h. A solution of 4-amino-N-(2-((tert-butyldimethylsilyl)oxy)ethyl)benzenesulfonamide (11.0 g, 33.3 mmol) in DCE (110 mL) and ^{t}BuOH (110 mL) was added dropwise followed by TEA (7.0 mL, 50.0 mmol) at 0° C. The reaction was stirred at 30° C. for 24 h. Solvents were removed and diluted with ethyl acetate and brine. The organic layer was separated and concentrated in vacuo. The resulting residue was purified by flash chromatography (eluting with 66% ethyl acetate in petroleum ether) to afford N-(2-((tert-butyldimethylsilyl) oxy) ethyl)-4-((4-chloro-5-(trifluoromethyl) pyrimidin-2-yl) amino) benzene sulfonamide as a light-yellow solid.

MS (ES-API positive): 512.1 (M+H)⁺.

### Step 4: 4-((4-(1H-pyrazol-1-yl)-5-(trifluoromethyl) pyrimidin-2-yl) amino)-N-(2-((tert-butyldimethylsilyl) oxy) ethyl) benzene sulfonamide

To a solution of N-(2-((tert-butyldimethylsilyl)oxy)ethyl)-4-((4-chloro-5-(trifluoromethyl)pyrimidin-2-yl)amino)benzenesulfonamide (13.7 g, 26.7 mmol) in DMF (50 mL) was added 1H-pyrazole (3.6 g, 53.5 mmol) and K₂CO₃ (11.1 g, 80.2 mmol) and stirred at 35° C. for 18 h. The reaction was diluted with ethyl acetate and brine. The organic layer was separated and concentrated in vacuo. The residue was purified by flash chromatography (eluting with 80%-100% ethyl acetate in petroleum ether) to afford the compound 4-((4-(1H-pyrazol-1-yl)-5-(trifluoromethyl) pyrimidin-2-yl) amino)-N-(2-((tert-butyldimethylsilyl) oxy) ethyl) benzene sulfonamide as a yellow solid.

MS (ES-API positive): 543.2 (M+H)⁺.

### Step 5: 4-((4-(1H-pyrazol-1-yl)-5-(trifluoromethyl) pyrimidin-2-yl) amino)-N-(2-hydroxyethyl) benzene sulfonamide

To a solution of 4-((4-(1H-pyrazol-1-yl)-5-(trifluoromethyl) pyrimidin-2-yl) amino)-N-(2-((tert-butyldimethylsilyl) oxy) ethyl) benzene sulfonamide (13.6 g, 25.1 mmol) in THF (100 mL) was added TBAF (62.9 mL, 62.9 mmol) and stirred at RT for 2 h. Solvents were removed in vacuo and the residue was diluted with ethyl acetate and brine. The organic layer was separated and concentrated in vacuo. The residue was purified by flash chromatography (eluting with 7% methanol in dichloromethane) to afford the crude compound 4-((4-(1H-pyrazol-1-yl)-5-(trifluoromethyl)pyrimidin-2-yl)amino)-N-(2-hydroxyethyl)benzene sulfonamide as a white solid.

MS (ES-API positive): 429.1 (M+H)⁺.

¹H NMR (400 MHz, DMSO-d₆) δ 10.86 (s, 1H), 9.01 (s, 1H), 8.62 (d, J=2.8 Hz, 1H), 8.13-7.88 (m, 3H), 7.80 (d, J=8.5 Hz, 2H), 7.50 (t, J=6.0 Hz, 1H), 6.71 (s, 1H), 4.70 (t, J=5.6 Hz, 1H), 3.40-3.38 (m, 2H), 2.80 (q, J=6.2 Hz, 2H).

### Example 42: 1,1-dioxo-N-[4-pyrazol-1-yl-5-(trifluoromethyl) pyrimidin-2-yl]-2,3-dihydro-1,2-benzothiazol-5-amine

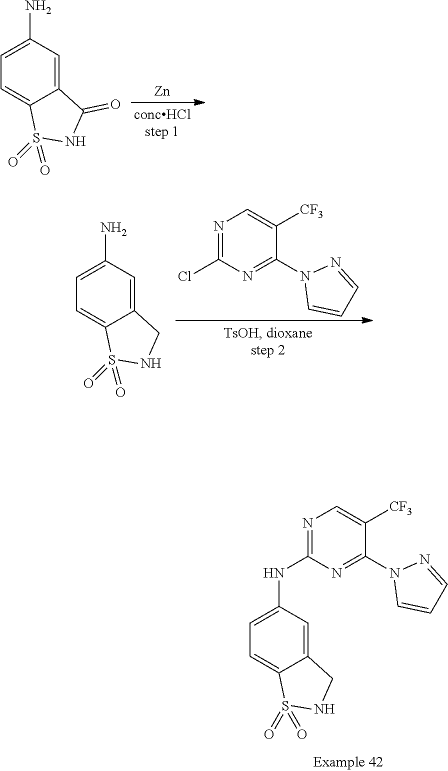

### Step 1: 1,1-dioxo-2,3-dihydro-1,2-benzothiazol-5-amine

To a mixture of 5-amino-1,1-dioxo-1,2-benzothiazol-3-one (500.0 mg, 2.5 mmol) in conc. HCl (5 mL) was added Zn (1.6 g, 25.0 mmol). The mixture was stirred at 25° C. for 2 h. Saturated NaHCO₃ solution was added until the pH to 7-8. The mixture was filtered and extracted with ethyl acetate (3×20 mL). The organic layers were washed with brine (30 mL), dried over Na₂SO₄ and concentrated to afford compound 1,1-dioxo-2,3-dihydro-1,2-benzothiazol-5-amine as a yellow solid without further purification.

MS (ES-API positive): 185.0 (M+H)⁺.

### Step 2: 1,1-dioxo-N-[4-pyrazol-1-yl-5-(trifluoromethyl) pyrimidin-2-yl]-2,3-dihydro-1,2-benzothiazol-5-amine

To a solution of 2-chloro-4-pyrazol-1-yl-5-(trifluoromethyl) pyrimidine (202.4 mg, 814.3 μmol) and 1,1-dioxo-2,3-dihydro-1,2-benzothiazol-5-amine (150.0 mg, 814.3 μmol) in dioxane (2 mL) was added TsOH (140.2 mg, 814.3 μmol). The mixture was stirred at 100° C. for 2 h, filtered and concentrated to give a residue. The residue was purified by prep-HPLC (eluting with 35%-55% acetonitrile in water). The crude product was triturated with DCM and MeOH (10 mL, v/v=5/1) at 25° C. for 20 min. The turbid solution was filtered, washed with DCM and MeOH (5 mL×3, v/v=5/1) to afford compound 1,1-dioxo-N-[4-pyrazol-1-yl-5-(trifluoromethyl) pyrimidin-2-yl]-2,3-dihydro-1,2-benzothiazol-5-amine as a white solid.

MS (ES-API positive): 396.9 (M+H)⁺.

¹H NMR (400 MHz, DMSO-d₆) δ 10.90-10.74 (m, 1H), 9.02 (s, 1H), 8.60-8.45 (m, 1H), 8.34-8.16 (m, 1H), 7.98 (s, 1H), 7.94-7.82 (m, 2H), 7.60-7.50 (m, 1H), 6.70 (s, 1H), 4.38 (d, J=5.0 Hz, 2H).

### Example 49: 4-[[4-[4-(1-cyano-1-methyl-ethyl) pyrazol-1-yl]-5-(trifluoromethyl) pyrimidin-2-yl]amino]-N-methyl-benzene sulfonamide

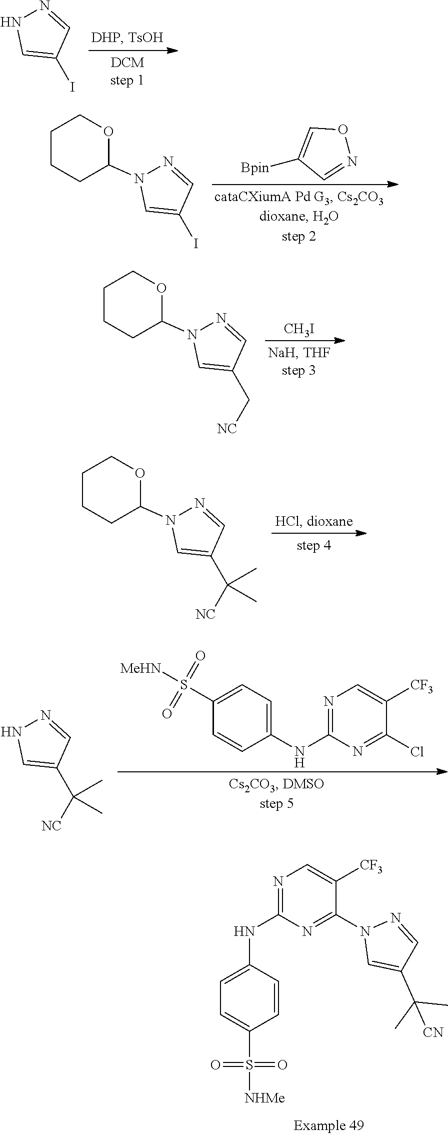

### Step 1: 4-iodo-1-tetrahydropyran-2-yl-pyrazole

To a solution of 4-iodo-1H-pyrazole (5.0 g, 25.8 mmol) and 3,4-dihydro-2H-pyran (4.8 g, 56.7 mmol) DCM (10 mL) was added 4-methylbenzenesulfonic acid (490.3 mg, 2.6 mmol) and stirred at 25° C. for 16 h. The reaction mixture was diluted with H₂O (40 mL) and extracted with ethyl acetate (60 mL×3). The combined organic layers were washed with brine, dried over Na₂SO₄, and concentrated. The residue was purified by flash chromatography (eluting with 0%-25% ethyl acetate in petroleum ether) to afford compound 4-iodo-1-tetrahydropyran-2-yl-pyrazole as a yellow oil.

MS (ES-API positive): 278.9 (M+H)⁺.

### Step 2: 2-(1-tetrahydropyran-2-ylpyrazol-4-yl) acetonitrile

To a mixture of 4-iodo-1-tetrahydropyran-2-yl-pyrazole (1.1 g, 4.0 mmol), 4-(4,4,5,5-tetramethyl-1,3,2-dioxaborolan-2-yl) isoxazole (925.7 mg, 4.8 mmol) and cataCXiumA Pd G3 (288.1 mg, 395.6 μmol) in H₂O (10 mL) and dioxane (30 mL) was added Cs₂CO₃ (3.9 g, 11.9 mmol). The mixture was stirred at 100° C. for 16 h. The reaction mixture was diluted with H₂O (20 mL) and extracted with ethyl acetate (20 mL×3). The combined organic layers were washed with brine, dried over Na₂SO₄ and concentrated. The residue was purified by flash chromatography (eluting with 0%-50% ethyl acetate in petroleum ether). Compound 2-(1-tetrahydropyran-2-ylpyrazol-4-yl) acetonitrile was obtained as a yellow oil.

MS (ES-API positive): 192.4 (M+H)⁺.

### Step 3: 2-methyl-2-(1-tetrahydropyran-2-ylpyrazol-4-yl) propane nitrile

To a solution of 2-(1-tetrahydropyran-2-ylpyrazol-4-yl) acetonitrile (200.0 mg, 1.1 mmol) in THF (3 mL) was added NaH (92.0 mg, 2.3 mmol, 60.0% purity) at 0° C. After completion of gas evolution, CH₃I (296.9 mg, 2.1 mmol) was added dropwise at 0° C. and stirred at 0-25° C. for 2 h. The reaction mixture was diluted with H₂O (20 mL) and extracted with ethyl acetate (20 mL×3). The combined organic layers were washed with brine, dried over Na₂SO₄, and concentrated. The residue was purified by flash chromatography (eluting with 0%-50% ethyl acetate in petroleum ether). Compound 2-methyl-2-(1-tetrahydropyran-2-ylpyrazol-4-yl) propane nitrile was obtained as a yellow oil.

MS (ES-API positive): 220.1 (M+H)⁺.

### Step 4: 2-methyl-2-(1H-pyrazol-4-yl) propane nitrile

To a solution of HCl in dioxane (2 mL) was added 2-methyl-2-(1-tetrahydropyran-2-ylpyrazol-4-yl) propane nitrile (120.0 mg, 547.2 μmol). The mixture was stirred at 50° C. for 2 h. The reaction mixture was concentrated in vacuo to give a residue as yellow oil without further purification.

MS (ES-API positive): 136.3 (M+H)⁺.

### Step 5: 4-[[4-[4-(1-cyano-1-methyl-ethyl) pyrazol-1-yl]-5-(trifluoromethyl) pyrimidin-2-yl]amino]-N-methyl-benzene sulfonamide

To a solution of 2-methyl-2-(1H-pyrazol-4-yl) propane nitrile (50.0 mg, 369.9 μmol) and 4-[[4-chloro-5-(trifluoromethyl) pyrimidin-2-yl]amino]-N-methyl-benzene sulfonamide (135.7 mg, 369.9 μmol) in DMSO (2 mL) was added Cs₂CO₃ (241.0 mg, 739.8 μmol). The mixture was stirred at 40° C. for 2 h. The reaction mixture was diluted with H₂O (20 mL) and extracted with ethyl acetate (20 mL×3). The combined organic layers were washed with brine, dried over Na₂SO₄, filtered, and concentrated. The residue was purified by prep-HPLC (eluting with 60%-80% acetonitrile in water). Compound 4-[[4-[4-(1-cyano-1-methyl-ethyl) pyrazol-1-yl]-5-(trifluoromethyl) pyrimidin-2-yl]amino]-N-methyl-benzene sulfonamide was obtained as a white solid.

MS (ES-API positive): 466.1 (M+H)⁺.

¹H NMR (400 MHz, Methanol-d₄) δ 8.92 (s, 1H), 8.63 (s, 1H), 8.04-7.92 (m, 3H), 7.82 (d, J=8.8 Hz, 2H), 2.54 (s, 3H), 1.77 (s, 6H).

### Example 51: 4-[[4-[4-(1-cyanocyclopropyl) pyrazol-1-yl]-5-(trifluoromethyl) pyrimidin-2-yl]aminol-N-methyl-benzene sulfonamide

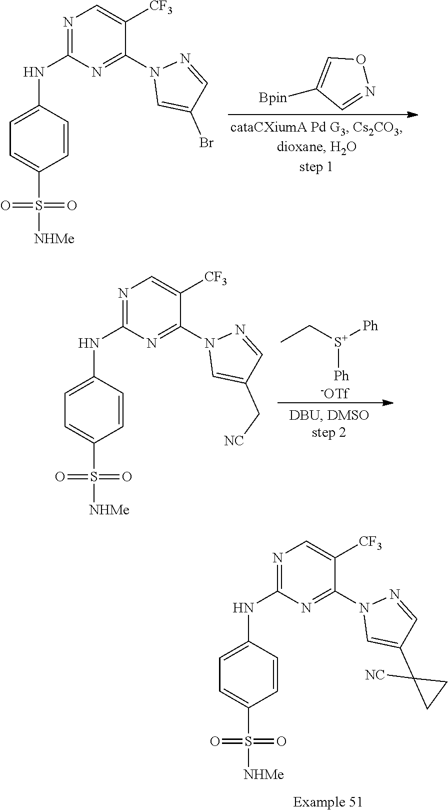

### Step 1: 4-[[4-[4-(cyanomethyl) pyrazol-1-yl]-5-(trifluoromethyl) pyrimidin-2-yl]amino]-N-methyl-benzene sulfonamide

To a mixture of 4-[[4-(4-iodopyrazol-1-yl)-5-(trifluoromethyl)pyrimidin-2-yl]amino]-N-methyl-benzenesulfonamide (1.0 g, 1.9 mmol), Cs₂CO₃ (1.9 g, 5.7 mmol) and 4-(4,4,5,5-tetramethyl-1,3,2-dioxaborolan-2-yl)isoxazole (446.4 mg, 2.3 mmol) in H₂O (4 mL) and dioxane (16 mL) was added cataCXiumA Pd G3 (138.9 mg, 190.8 μmol). The mixture was stirred at 100° C. for 16 h under N₂. The reaction mixture was added to H₂O (30 mL) and extracted with ethyl acetate (20 mL×3). The combined organic layers were washed with brine, dried over Na₂SO₄, filtered, and concentrated. The residue was purified by flash chromatography (eluting with 0%-100% ethyl acetate in petroleum ether). Compound 4-[[4-[4-(cyanomethyl) pyrazol-1-yl]-5-(trifluoromethyl) pyrimidin-2-yl]amino]-N-methyl-benzene sulfonamide was obtained as a brown solid.

MS (ES-API positive): 437.9 (M+H)⁺.

### Step 2: 4-[[4-[4-(1-cyanocyclopropyl) pyrazol-1-yl]-5-(trifluoromethyl) pyrimidin-2-yl]amino]-N-methyl-benzene sulfonamide

To a solution of 4-[[4-[4-(cyanomethyl) pyrazol-1-yl]-5-(trifluoromethyl) pyrimidin-2-yl]amino]-N-methyl-benzene sulfonamide (150.0 mg, 342.9 μmol) in DMSO (1.8 mL) was added diphenyl(vinyl)sulfonium trifluoromethanesulfonate (150.0 mg, 413.9 μmol). The mixture was stirred at 20° C. for 2 min. DBU (157.5 mg, 1.0 mmol) was added and stirred at 20° C. for another 18 h. The reaction mixture was added to H₂O (20 mL) and extracted with ethyl acetate (20 mL×3). The combined organic layers were washed with brine, dried over Na₂SO₄, filtered, and concentrated. The residue was purified by prep-HPLC (eluting with 42%-62% acetonitrile in water). Compound 4-[[4-[4-(1-cyanocyclopropyl) pyrazol-1-yl]-5-(trifluoromethyl) pyrimidin-2-yl]amino]-N-methyl-benzene sulfonamide was obtained as yellow solid.

MS (ES-API positive): 464.1 (M+H)⁺.

¹H NMR (400 MHz, Methanol-d₄) δ 8.93 (s, 1H), 8.65-8.60 (m, 1H), 8.02-7.97 (m, 2H), 7.87-7.83 (m, 2H), 7.82-7.79 (m, 1H), 2.62-2.52 (m, 3H), 1.80-1.72 (m, 2H), 1.58-1.43 (m, 2H).

### Example 52 and Example 53: 4-[[4-[4-(2,2-difluorocyclopropyl)pyrazol-1-yl]-5-(trifluoromethyl)pyrimidin-2-yl]amino]-N-methyl-benzenesulfonamide

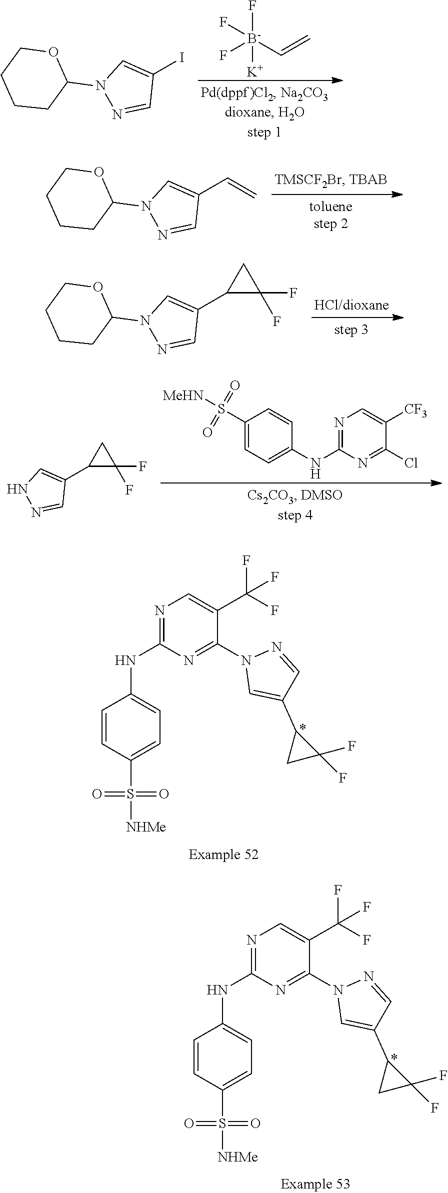

### Step 1: 1-tetrahydropyran-2-yl-4-vinyl-pyrazole

To a mixture of 4-iodo-1-tetrahydropyran-2-yl-pyrazole (3.2 g, 11.5 mmol) and potassium trifluoro(vinyl)boranuide (2.3 g, 17.3 mmol) in dioxane (30 mL) and H₂O (3 mL) was added Na₂CO₃ (3.7 g, 34.5 mmol) and Pd(dppf)Cl₂ (1.3 g, 1.7 mmol). The mixture was stirred at 100° C. for 2 h. The reaction mixture was poured into H₂O (40 ml) and extracted with ethyl acetate (30 mL×3). The combined organic layers were washed with brine, dried over Na₂SO₄, filtered, and concentrated. The residue was purified by flash chromatography (eluting with 0%-35% ethyl acetate in petroleum ether). Compound 1-tetrahydropyran-2-yl-4-vinyl-pyrazole was obtained as yellow oil.

MS (ES-API positive): 178.2 (M+H)⁺.

### Step 2: 4-(2,2-difluorocyclopropyl)-1-tetrahydropyran-2-yl-pyrazole

To a solution of 1-tetrahydropyran-2-yl-4-vinyl-pyrazole (790.0 mg, 4.4 mmol) in toluene (8 mL) was added [bromo(difluoro)methyl]-trimethyl-silane (1.1 g, 5.3 mmol) and TBAB (142.9 mg, 443.2 μmol). The mixture was stirred at 110° C. for 1 h. Then [bromo(difluoro) methyl]-trimethyl-silane (1.1 g, 5.3 mmol) was added. The mixture was stirred at 110° C. for another 1 h. The reaction mixture was poured into H₂O (10 ml) and extracted with ethyl acetate (10 mL×3). The combined organic layers were washed with brine, dried over Na₂SO₄, filtered, and concentrated. The residue was purified by flash chromatography (eluting with 0%-35% ethyl acetate in petroleum ether). Compound 4-(2,2-difluorocyclopropyl)-1-tetrahydropyran-2-yl-pyrazole was obtained as yellow oil.

MS (ES-API positive): 228.2 (M+H)⁺.

### Step 3: 4-(2,2-difluorocyclopropyl)-1H-pyrazole

A mixture of 4-(2,2-difluorocyclopropyl)-1-tetrahydropyran-2-yl-pyrazole (400.0 mg, 1.8 mmol) in HCl (4 M in dioxane, 0.4 mL) was stirred at 25° C. for 1 h. The reaction mixture was concentrated and purified by flash chromatography (eluting with 0%-25% ethyl acetate in petroleum ether). Compound 4-(2,2-difluorocyclopropyl)-1H-pyrazole was obtained as a white solid.

MS (ES-API positive): 144.1 (M+H)⁺.

### Step 4: 4-[[4-[4-(2,2-difluorocyclopropyl)pyrazol-1-yl]-5-(trifluoromethyl)pyrimidin-2-yl]amino]-N-methyl-benzenesulfonamide

To a solution of 4-(2,2-difluorocyclopropyl)-1H-pyrazole (250.0 mg, 1.7 mmol) and 4-[[4-chloro-5-(trifluoromethyl) pyrimidin-2-yl]amino]-N-methyl-benzene sulfonamide (954.3 mg, 2.6 mmol) in DMSO (4 mL) was added Cs₂CO₃ (1.7 g, 5.2 mmol). The mixture was stirred at 80° C. for 2 h. The reaction mixture was poured into water (20 ml) and extracted with ethyl acetate (15 mL×3). The combined organic layers were washed with brine, dried over Na₂SO₄, filtered, and concentrated. The residue was purified by SFC (column: DAICEL CHIRALPAK IG (250 mm×30 mm×10 am); [CO₂-MeOH (0.1% NH₃—H₂O)]; B %: 40%) to give Example 52 (retention time: 1.373 min) and Example 53 (retention time: 1.716 min).

Example 52 and Example 53 were obtained as a white solid after purification by pre-HPLC (eluting with 55%-75% acetonitrile in water).

MS (ES-API positive): 474.4 (M+H)⁺.

Example 52: ¹H NMR (400 MHz, Methanol-d₄) δ 8.91 (s, 1H), 8.50 (s, 1H), 7.98 (d, J=8.8 Hz, 2H), 7.90-7.75 (m, 3H), 2.83 (dt, J=7.7, 12.2 Hz, 1H), 2.56 (s, 3H), 2.04-1.91 (m, 1H), 1.52-1.47 (m, 1H).

Example 53: ¹H NMR (400 MHz, Methanol-d₄) δ 8.91 (s, 1H), 8.50 (s, 1H), 7.98 (d, J=8.8 Hz, 2H), 7.90-7.75 (m, 3H), 2.83 (dt, J=7.7, 12.2 Hz, 1H), 2.56 (s, 3H), 2.04-1.91 (m, 1H), 1.52-1.47 (m, 1H).

### Example 72: 4-[[4-[4-(2-hydroxy-1,1,2-trimethyl-propyl) pyrazol-1-yl]-5-(trifluoromethyl) pyrimidin-2-yl]amino]-N-methyl-benzene sulfonamide

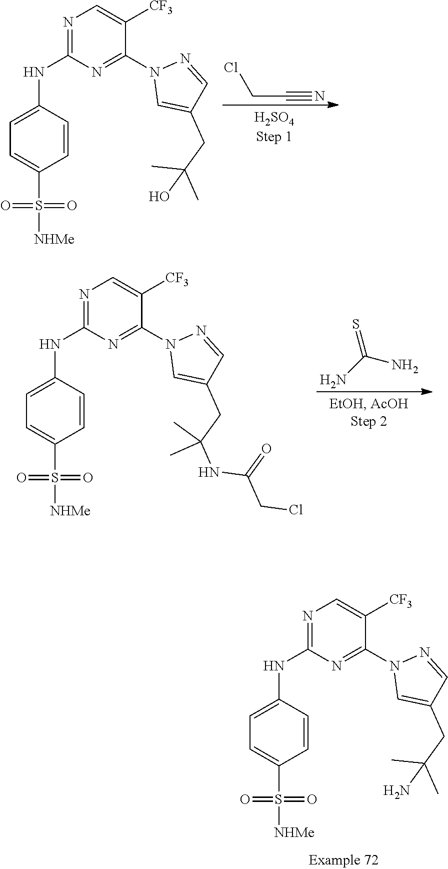

### Step 1: 2-chloro-N-[1,1-dimethyl-2-[1-[2-[4-(methylsulfamoyl) anilino]-5-(trifluoromethyl) pyrimidin-4-yl]pyrazol-4-yl]ethyl]acetamide

To a solution of 4-[[4-[4-(2-hydroxy-2-methyl-propyl) pyrazol-1-yl]-5-(trifluoromethyl) pyrimidin-2-yl]amino]-N-methyl-benzene sulfonamide (85.0 mg, 180.7 μmol) in 2-chloroacetonitrile (0.9 mL) was added H₂SO₄ (8.0 mmol, 0.4 mL) dropwise at 0° C. The mixture was stirred at 20° C. for 1 h. The reaction was treated with ice water (8 mL) and aqueous NaHCO₃ to pH about 8. The mixture was extracted with ethyl acetate (10 mL×3). The combined organic layers were dried over Na₂SO₄, filtered, and concentrated to give the crude compound 2-chloro-N-[1,1-dimethyl-2-[1-[2-[4-(methylsulfamoyl) anilino]-5-(trifluoromethyl) pyrimidin-4-yl]pyrazol-4-yl]ethyl]acetamide as a white solid without further purification.

MS (ES-API positive): 546.2 (M+H)⁺.

### Step 2: 4-[[4-[4-(2-amino-2-methyl-propyl) pyrazol-1-yl]-5-(trifluoromethyl) pyrimidin-2-yl]amino]-N-methyl-benzene sulfonamide

To a solution of 2-chloro-N-[1,1-dimethyl-2-[1-[2-[4-(methylsulfamoyl) anilino]-5-(trifluoromethyl) pyrimidin-4-yl]pyrazol-4-yl]ethyl]acetamide (90.0 mg, 164.8 μmol) in EtOH (6 mL) and AcOH (1.0 mL) was added thiourea (25.1 mg, 329.7 μmol). The mixture was stirred at 85° C. for 1 h. The reaction mixture was filtered and concentrated. The residue was purified by prep-HPLC (eluting with 23%-43% acetonitrile in water). Compound 4-[[4-[4-(2-amino-2-methyl-propyl) pyrazol-1-yl]-5-(trifluoromethyl) pyrimidin-2-yl]amino]-N-methyl-benzene sulfonamide was obtained as a white solid.

MS (ES-API positive): 470.1 (M+H)⁺.

¹H NMR (400 MHz, Methanol-d₄) δ 8.91 (s, 1H), 8.51 (s, 1H), 8.00-7.89 (m, 2H), 7.87-7.76 (m, 3H), 2.91 (s, 2H), 2.54 (s, 3H), 1.40 (s, 6H).

### Example 74 and Example 75: 4-[[4-[4-[(1S,2R)-2-hydroxycyclopentyl]pyrazol-1-yl]-5-(trifluoromethyl)pyrimidin-2-yl]amino]-N-methyl-benzenesulfonamide and 4-[[4-[4-[(1R,2S)-2-hydroxycyclopentyl]pyrazol-1-yl]-5-(trifluoromethyl)pyrimidin-2-yl]amino]-N-methyl-benzenesulfonamide

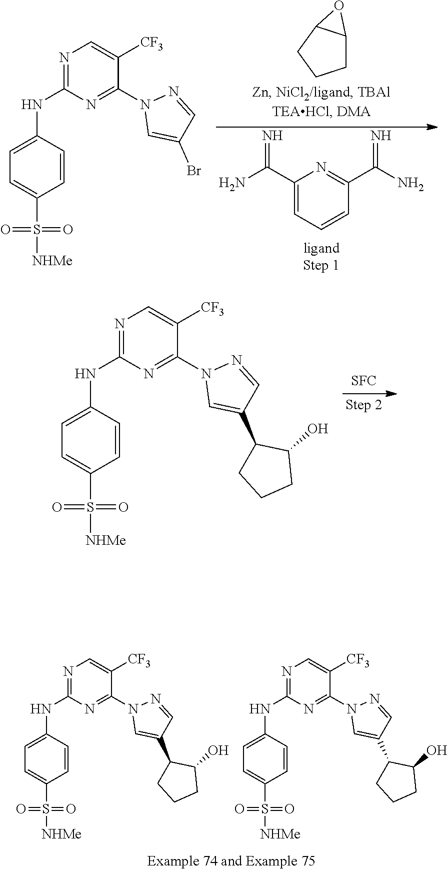

### Step 1: 4-[[4-[4-[trans-2-hydroxycyclopentyl]pyrazol-1-yl]-5-(trifluoromethyl)pyrimidin-2-yl]amino]-N-methyl-benzenesulfonamide

To a mixture of pyridine-2,6-dicarboxamidine (34.2 mg, 209.5 μmol), 4-[[4-(4-bromopyrazol-1-yl)-5-(trifluoromethyl)pyrimidin-2-yl]amino]-N-methyl-benzene sulfonamide (500.0 mg, 1.1 mmol) and 6-oxabicyclo[3.1.0]hexane (176.3 mg, 2.1 mmol) in DMA (10 mL) was added Zn (274.0 mg, 4.2 mmol), TBAI (580.5 mg, 1.6 mmol), diiodonickel (65.5 mg, 209.5 μmol) and N,N-diethylethanamine hydrochloride (144.2 mg, 1.1 mmol). The mixture was stirred at 80° C. for 4 h under N₂. The mixture was poured into water (50 mL) and extracted with ethyl acetate (50 mL×3). The combined organic phases were washed with brine, dried over Na₂SO₄ and concentrated. The residue was purified by flash chromatography (eluting with 0%-70% ethyl acetate in petroleum ether). Compound 4-[[4-[4-[trans-2-hydroxycyclopentyl]pyrazol-1-yl]-5-(trifluoromethyl)pyrimidin-2-yl]amino]-N-methyl-benzenesulfonamide was obtained as a white solid.

MS (ES-API positive): 483.1 (M+H)⁺.

### Step 2: 4-[[4-[4-[(1S,2R)-2-hydroxycyclopentyl]pyrazol-1-yl]-5-(trifluoromethyl)pyrimidin-2-yl]amino]-N-methyl-benzenesulfonamide & 4-[[4-[4-[(1R,2S)-2-hydroxycyclopentyl]pyrazol-1-yl]-5-(trifluoromethyl)pyrimidin-2-yl]amino]-N-methyl-benzenesulfonamide

The residue was purified by SFC (column: DAICEL CHIRALPAK IC (250 mmx 30 mm×10 am); [CO₂-EtOH (0.1% NH₃H₂O)]; B %: 40%). Example 74 (retention time: 2.294 min) and Example 75 (retention time: 3.013 min) were obtained as a white solid.

MS (ES-API positive): 483.1 (M+H)⁺.

Example 74: ¹H NMR (400 MHz, CD₃OD) δ 8.87 (s, 1H), 8.43 (s, 1H), 7.97 (d, J=8.8 Hz, 2H), 7.89-7.76 (m, 3H), 4.07 (q, J=6.8 Hz, 1H), 2.92 (q, J=7.8 Hz, 1H), 2.55 (s, 3H), 2.30-2.17 (m, 1H), 2.13-2.00 (m, 1H), 1.95-1.63 (m, 4H).

Example 75: ¹H NMR (400 MHz, CD₃OD) δ 8.86 (s, 1H), 8.42 (s, 1H), 7.96 (d, J=8.9 Hz, 2H), 7.87-7.74 (m, 3H), 4.07 (q, J=6.8 Hz, 1H), 2.92 (q, J=7.8 Hz, 1H), 2.26-2.14 (m, 1H), 2.13-2.01 (m, 1H), 1.93-1.62 (m, 4H).

### Example 76 and Example 77: 4-((4-(4-((1S,2S)-2-hydroxycyclopentyl)-1H-pyrazol-1-yl)-5-(trifluoromethyl) pyrimidin-2-yl) amino)-N-methylbenzene sulfonamide and 4-((4-(4-((1R,2R)-2-hydroxycyclopentyl)-1H-pyrazol-1-yl)-5-(trifluoromethyl) pyrimidin-2-yl) amino)-N-methylbenzene sulfonamide

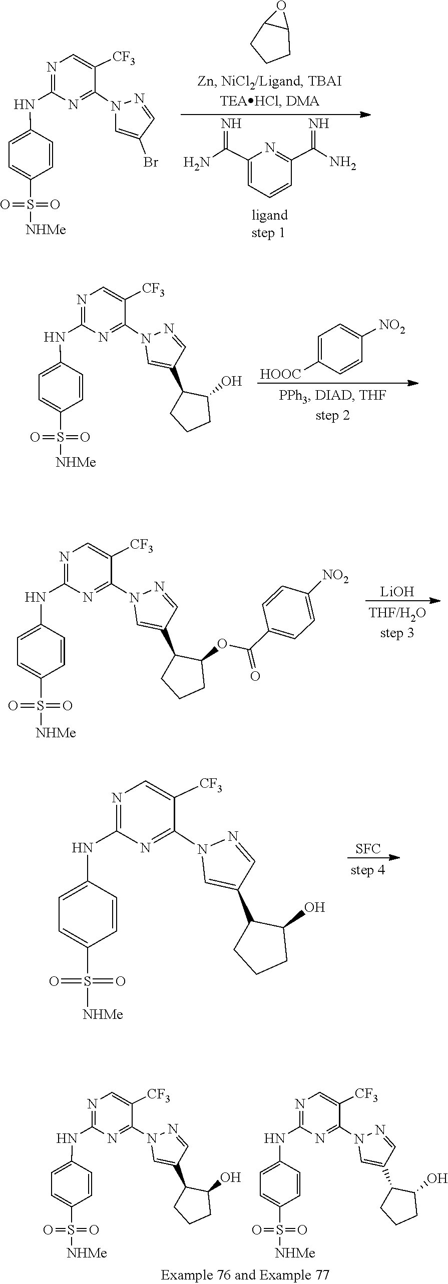

### Step 1: 4-((4-(4-(2-hydroxycyclopentyl)-1H-pyrazol-1-yl)-5-(trifluoromethyl)pyrimidin-2-yl)amino)-N-methylbenzenesulfonamide

Same as step 1 for Examples 74 and 75.

### Step 2: 2-(1-(2-((4-(N-methylsulfamoyl)phenyl)amino)-5-(trifluoromethyl)pyrimidin-4-yl)-1H-pyrazol-4-yl)cyclopentyl 4-nitrobenzoate

To a solution of 4-nitrobenzoic acid (69.3 mg, 414.5 μmol) and 4-[[4-[4-(2-hydroxycyclopentyl) pyrazol-1-yl]-5-(trifluoromethyl) pyrimidin-2-yl]amino]-N-methyl-benzene sulfonamide (200.0 mg, 414.5 μmol) in THF (3 mL) was added PPh₃ (326.2 mg, 1.2 mmol) and DIAD (251.5 mg, 1.2 mmol). The mixture was stirred at 25° C. for 2 h. The mixture was poured into water (2 mL) and extracted with ethyl acetate (2 mL×3). The combined organic phases were washed with brine, dried over Na₂SO₄, and concentrated. The residue was purified by flash chromatography (eluting with 0%-50% ethyl acetate in petroleum ether). Compound [2-[1-[2-[4-(methylsulfamoyl) anilino]-5-(trifluoromethyl) pyrimidin-4-yl]pyrazol-4-yl]cyclopentyl]4-nitrobenzoate was obtained as a white solid.

MS (ES-API positive): 632.2 (M+H)⁺.

### Step 3: 4-((4-(4-(2-hydroxycyclopentyl)-1H-pyrazol-1-yl)-5-(trifluoromethyl) pyrimidin-2-yl) amino)-N-methylbenzene sulfonamide & 4-((4-(4-(2-hydroxycyclopentyl)-1H-pyrazol-1-yl)-5-(trifluoromethyl) pyrimidin-2-yl) amino)-N-methylbenzene sulfonamide

To a solution of [2-[1-[2-[4-(methylsulfamoyl) anilino]-5-(trifluoromethyl) pyrimidin-4-yl]pyrazol-4-yl]cyclopentyl]4-nitrobenzoate (180.0 mg, 285.0 μmol) in THF (2 mL) and H₂O (0.4 mL) was added LiOH·H₂O (59.8 mg, 1.4 mmol). The mixture was stirred at 25° C. for 2 h. The reaction mixture was diluted with H₂O (10 mL) and extracted with ethyl acetate (10 mL×3). The combined organic layers were washed with brine, dried over Na₂SO₄, and concentrated. The residue was purified by prep-HPLC (eluting with 38%-58% acetonitrile in water).

MS (ES-API positive): 483.2 (M+H)⁺.

Step 4: The product was separated by SFC (column: DAICEL CHIRALPAK AD (250 mm×30 mm×10 am); [CO₂-EtOH (0.1% NH₃H₂O)]; B %: 60%). Example 76 (retention time: 1.055 min) and Example 77 (retention time: 2.025 min) were obtained as a white solid.

MS (ES-API positive): 483.2 (M+H)⁺.

Example 76: ¹H NMR (400 MHz, Methanol-d₄) δ 8.76 (s, 1H), 8.35 (s, 1H), 7.87 (d, J=8.8 Hz, 2H), 7.75-7.70 (m, 2H), 7.69 (s, 1H), 4.19-4.12 (m, 1H), 2.95-2.83 (m, 1H), 2.02-1.79 (m, 4H), 1.76-1.59 (m, 2H)

Example 77: ¹H NMR (400 MHz, Methanol-d₄) δ 8.88 (s, 1H), 8.47 (s, 1H), 7.99 (d, J=8.7 Hz, 2H), 7.87-7.83 (m, 2H), 7.81 (s, 1H), 4.27 (s, 1H), 3.07-2.98 (m, 1H), 2.13-1.92 (m, 4H), 1.87-1.73 (m, 2H).

Example 78 and Example 79: 4-((4-(4-((1R,3R)-3-hydroxycyclopentyl)-1H-pyrazol-1-yl)-5-(trifluoromethyl)pyrimidin-2-yl)amino)-N-methylbenzenesulfonamide and 4-((4-(4-((1S,3S)-3-hydroxycyclopentyl)-1H-pyrazol-1-yl)-5-(trifluoromethyl)pyrimidin-2-yl)amino)-N-methylbenzenesulfonamide

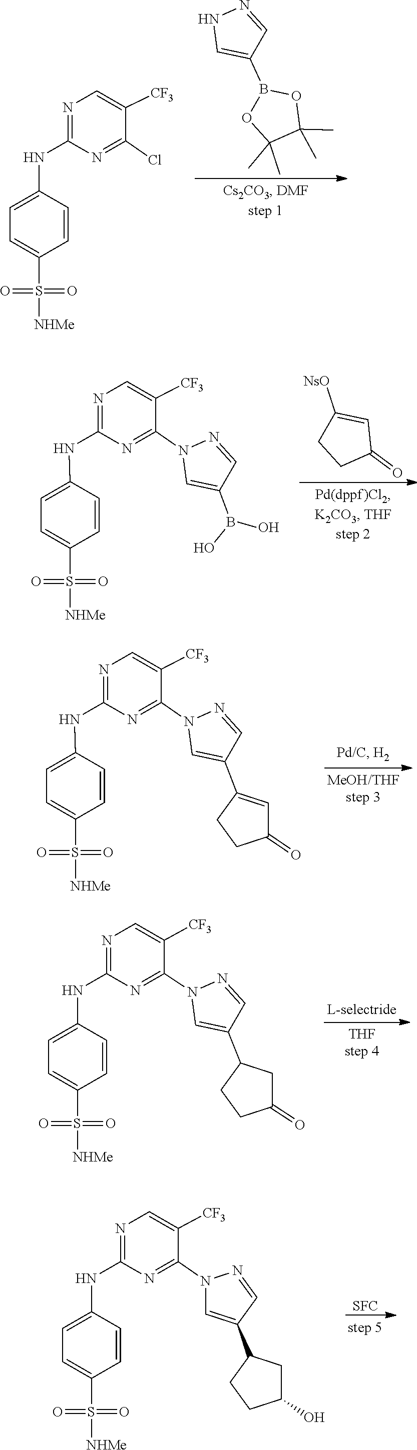

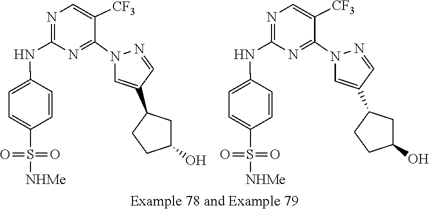

### Step 1: [1-[2-[4-(methylsulfamoyl) anilino]-5-(trifluoromethyl) pyrimidin-4-yl]pyrazol-4-yl]boronic acid

To a solution of 4-(4,4,5,5-tetramethyl-1,3,2-dioxaborolan-2-yl)-1H-pyrazole (1.2 g, 6.0 mmol) in DMF (30 mL) was added Cs₂CO₃ (3.6 g, 10.9 mmol) and 4-[[4-chloro-5-(trifluoromethyl) pyrimidin-2-yl]amino]-N-methyl-benzene sulfonamide (2.0 g, 5.5 mmol) at 20° C. The mixture was stirred at 20° C. for 60 h. The reaction mixture was poured into H₂O (150 mL). The pH was adjusted to 7.0 by 2 N aq. HCl. The crude was filtered to give [1-[2-[4-(methylsulfamoyl) anilino]-5-(trifluoromethyl) pyrimidin-4-yl]pyrazol-4-yl]boronic acid as a light-yellow solid which was used to next step without purification.

MS (ES-API positive): 443.0 (M+H)⁺.

### Step 2: N-methyl-4-[[4-[4-(3-oxocyclopenten-1-yl) pyrazol-1-yl]-5-(trifluoromethyl) pyrimidin-2-yl]amino]benzene sulfonamide

To a mixture of [1-[2-[4-(methylsulfamoyl)anilino]-5-(trifluoromethyl)pyrimidin-4-yl]pyrazol-4-yl]boronic acid (2.5 g, 4.0 mmol) and K₂CO₃ (2 M, 7.1 mL) in THF (21.2 mL) was added (3-oxocyclopenten-1-yl) 4-nitrobenzenesulfonate (1.0 g, 3.5 mmol) and Pd(dppf)Cl₂ (103.3 mg, 141.2 μmol). The mixture was stirred at 30° C. for 16 h under N₂. The reaction mixture was poured into H₂O (30 mL) and extracted with ethyl acetate (20 mL×3). The organic phase was washed with brine, dried over Na₂SO₄, filtered, and concentrated. The residue was purified by flash chromatography (eluting with 0%-65% ethyl acetate in petroleum ether). Compound N-methyl-4-[[4-[4-(3-oxocyclopenten-1-yl) pyrazol-1-yl]-5-(trifluoromethyl) pyrimidin-2-yl]amino]benzene sulfonamide was obtained as a light yellow solid.

MS (ES-API positive): 479.0 (M+H)⁺.

### Step 3: N-methyl-4-[[4-[4-(3-oxocyclopentyl) pyrazol-1-yl]-5-(trifluoromethyl) pyrimidin-2-yl]amino]benzene sulfonamide

To a solution of N-methyl-4-[[4-[4-(3-oxocyclopenten-1-yl)pyrazol-1-yl]-5-(trifluoromethyl)pyrimidin-2-yl]amino]benzenesulfonamide (600.0 mg, 1.2 mmol) in MeOH (30 mL) and THF (30 mL) was added Pd/C (253.6 mg, 238.3 μmol, 10.0% purity) and stirred at 40° C. for 6 h under H₂ (40 psi). The reaction mixture was filtered and concentrated to give N-methyl-4-[[4-[4-(3-oxocyclopentyl) pyrazol-1-yl]-5-(trifluoromethyl) pyrimidin-2-yl]amino]benzene sulfonamide as a light yellow solid which was used directly without further purification.

MS (ES-API positive): 481.0 (M+H)⁺.

### Step 4: 4-[[4-[4-(3-hydroxycyclopentyl)pyrazol-1-yl]-5-(trifluoromethyl)pyrimidin-2-yl]amino]-N-methyl-benzenesulfonamide

To a solution of N-methyl-4-[[4-[4-(3-oxocyclopentyl) pyrazol-1-yl]-5-(trifluoromethyl) pyrimidin-2-yl]amino]benzene sulfonamide (130.0 mg, 270.6 μmol) in THF (4 mL) was added L-Selectride (1 M, 541.2 μL) at −78° C. and stirred for 1 h under N₂. The reaction mixture was quenched with H₂O (8 mL), extracted with ethyl acetate, washed with brine, and concentrated. The residue was purified by flash chromatography (eluting with 60% ethyl acetate in petroleum ether). Compound 4-[[4-[4-(3-hydroxycyclopentyl) pyrazol-1-yl]-5-(trifluoromethyl) pyrimidin-2-yl]amino]-N-methyl-benzene sulfonamide was obtained as a white solid.

MS (ES-API positive): 483.2 (M+H)⁺.

### Step 5: 4-[[4-[4-(3-hydroxycyclopentyl)pyrazol-1-yl]-5-(trifluoromethyl)pyrimidin-2-yl]amino]-N-methyl-benzenesulfonamide

SFC (column: DAICEL CHIRALPAK IG (250 mm×30 mm×10 am); [CO₂-EtOH (0.1% NH₃—H₂O)]; B %: 45%) to give Example 78 (retention time: 1.779 min) and Example 79 (retention time: 2.740 min) as a white solid.

MS (ES-API positive): 483.1 (M+H)*.

Example 78: ¹H NMR (400 MHz, Methanol-d₄) δ 8.86 (s, 1H), 8.36 (s, 1H), 8.01-7.92 (m, 2H), 7.84-7.79 (m, 2H), 7.75 (s, 1H), 4.53-4.41 (m, J=2.6, 5.7 Hz, 1H), 3.40-3.33 (m, 1H), 2.54 (s, 3H), 2.33-2.22 (m, 1H), 2.21-2.06 (m, 2H), 1.84-1.80 (m, 1H), 1.75-1.54 (m, 2H).

Example 79: ¹H NMR (400 MHz, Methanol-d₄) δ 8.86 (s, 1H), 8.35 (s, 1H), 7.96 (d, J=8.8 Hz, 2H), 7.86-7.79 (m, 2H), 7.75 (s, 1H), 4.45-4.41 (m, 1H), 3.41-3.34 (m, 1H), 2.54 (s, 3H), 2.35-2.22 (m, 1H), 2.21-2.06 (m, 2H), 1.84-1.80 (m, 1H), 1.77-1.55 (m, 2H).

### Example 80: N-methyl-4-[[4-[4-(1-methyl-4-piperidyl) pyrazol-1-yl]-5-(trifluoromethyl) pyrimidin-2-yl]amino]benzene sulfonamide

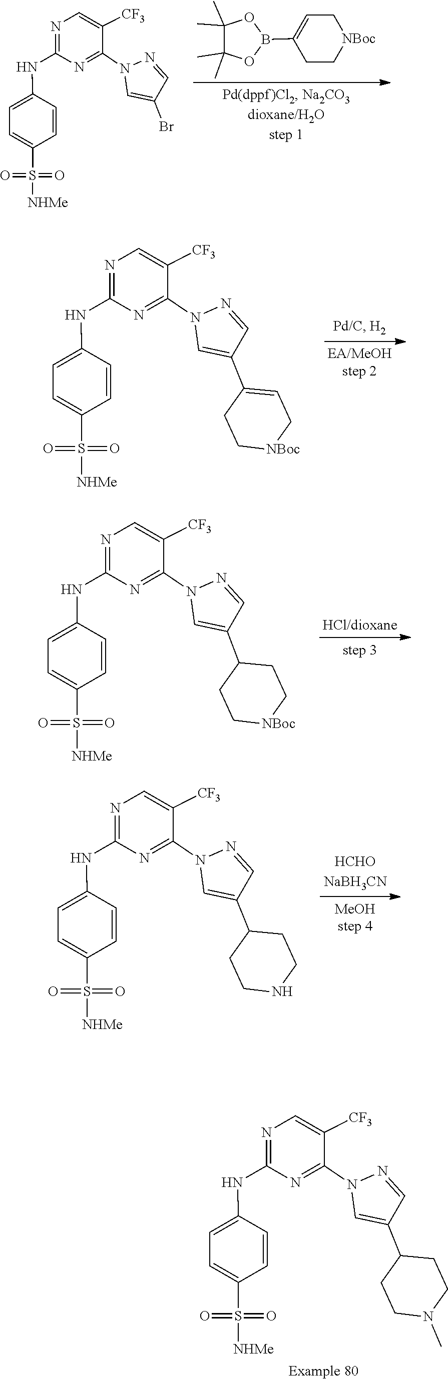

### Step 1: tert-butyl 4-(1-(2-((4-(N-methylsulfamoyl) phenyl) amino)-5-(trifluoromethyl) pyrimidin-4-yl)-1H-pyrazol-4-yl)-3,6-dihydropyridine-1(2H)-carboxylate

To a mixture of tert-butyl 4-(4,4,5,5-tetramethyl-1,3,2-dioxaborolan-2-yl)-3,6-dihydro-2H-pyridine-1-carboxylate (388.7 mg, 1.3 mmol) and 4-[[4-(4-bromopyrazol-1-yl)-5-(trifluoromethyl)pyrimidin-2-yl]amino]-N-methyl-benzenesulfonamide (500.0 mg, 1.0 mmol) in dioxane (10 mL) and H₂O (2 mL) was added Na₂CO₃ (222.1 mg, 2.1 mmol) and Pd(dppf)Cl₂ (76.7 mg, 104.8 μmol). The mixture was degassed and purged with N₂ for 3 times and stirred at 80° C. for 16 h. The reaction mixture was diluted with water and extracted with ethyl acetate. The combined organic layers were dried over Na₂SO₄, filtered, and concentrated. The crude product tert-butyl 4-[1-[2-[4-(methylsulfamoyl)anilino]-5-(trifluoromethyl)pyrimidin-4-yl]pyrazol-4-yl]-3,6-dihydro-2H-pyridine-1-carboxylate as a brown solid was used for the next step without further purification.

MS (ES-API positive): 580.2 (M+H)⁺.

### Step 2: tert-butyl 4-(1-(2-((4-(N-methylsulfamoyl) phenyl) amino)-5-(trifluoromethyl) pyrimidin-4-yl)-1H-pyrazol-4-yl) piperidine-1-carboxylate

To a solution of tert-butyl 4-[1-[2-[4-(methylsulfamoyl)anilino]-5-(trifluoromethyl)pyrimidin-4-yl]pyrazol-4-yl]-3,6-dihydro-2H-pyridine-1-carboxylate (320.0 mg, 552.1 μmol) in MeOH (10 mL) and ethyl acetate (10 mL) was added dihydroxy palladium (387.7 mg, 552.1 μmol, 20.0% purity). The mixture was stirred at 20° C. for 16 h under H₂ (50 psi). The reaction mixture was filtered and concentrated. The crude product tert-butyl 4-[1-[2-[4-(methylsulfamoyl)anilino]-5-(trifluoromethyl)pyrimidin-4-yl]pyrazol-4-yl]piperidine-1-carboxylate as a brown solid was used for the next step without further purification.

MS (ES-API positive): 582.2 (M+H)⁺.

### Step 3: N-methyl-4-[[4-[4-(4-piperidyl) pyrazol-1-yl]-5-(trifluoromethyl) pyrimidin-2-yl]amino]benzene sulfonamide

A solution of tert-butyl 4-[1-[2-[4-(methylsulfamoyl) anilino]-5-(trifluoromethyl) pyrimidin-4-yl]pyrazol-4-yl]piperidine-1-carboxylate (265.0 mg, 455.6 μmol) in dioxane (1 mL) was added HCl (4 M in dioxane, 1.1 mL). The mixture was degassed and purged with N₂ for 3 times, and stirred at 20° C. for 2 h. The reaction mixture was filtered and concentrated. The crude product N-methyl-4-[[4-[4-(4-piperidyl)pyrazol-1-yl]-5-(trifluoromethyl)pyrimidin-2-yl]amino]benzene sulfonamide as a brown solid was used for the next step without further purification.

MS (ES-API positive): 482.2 (M+H)⁺.

### Step 4: N-methyl-4-[[4-[4-(1-methyl-4-piperidyl) pyrazol-1-yl]-5-(trifluoromethyl) pyrimidin-2-yl]amino]benzene sulfonamide

To a solution of N-methyl-4-[[4-[4-(4-piperidyl) pyrazol-1-yl]-5-(trifluoromethyl) pyrimidin-2-yl]amino]benzene sulfonamide (120.0 mg, 249.2 μmol) in MeOH (1 mL) was added NaBH₃CN (47.0 mg, 747.7 μmol) and formaldehyde (60.7 mg, 747.7 μmol, 37.0% purity). The mixture was stirred at 20° C. for 1 h. The reaction mixture was filtered and concentrated. The residue was purified by prep-HPLC (eluting with 33%-63% acetonitrile in water). Compound N-methyl-4-[[4-[4-(1-methyl-4-piperidyl)pyrazol-1-yl]-5-(trifluoromethyl)pyrimidin-2-yl]amino]benzene sulfonamide was obtained as a white solid.

MS (ES-API positive): 496.1 (M+H)⁺.

¹H NMR (400 MHz, Methanol-d₄) δ 8.87 (s, 1H), 8.41 (s, 1H), 7.96 (d, J=8.7 Hz, 2H), 7.83 (d, J=7.4 Hz, 3H), 3.82 (d, J=12.2 Hz, 2H), 2.95-2.85 (m, 5H), 2.83-2.75 (m, 1H), 2.54 (s, 3H), 2.16-2.09 (m, 2H), 1.83-1.67 (m, 2H).

### Example 87: N-methyl-4-[[4-(4-pyrrolidin-1-ylpyrazol-1-yl)-5-(trifluoromethyl) pyrimidin-2-yl]amino]benzene sulfonamide

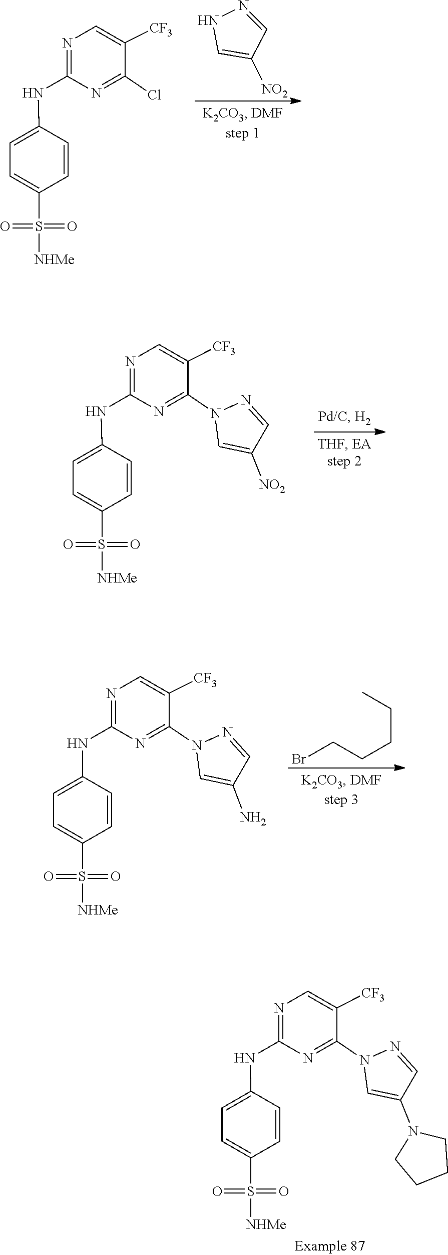

### Step 1: N-methyl-4-[[4-(4-nitropyrazol-1-yl)-5-(trifluoromethyl) pyrimidin-2-yl]amino]benzene sulfonamide

To a mixture of 4-[[4-chloro-5-(trifluoromethyl) pyrimidin-2-yl]amino]-N-methyl-benzene sulfonamide (2.0 g, 5.5 mmol) and 4-nitro-1H-pyrazole (616.6 mg, 5.5 mmol) in DMF (30 mL) was added Cs₂CO₃ (3.6 g, 11.0 mmol). The mixture was stirred at 25° C. for 2 h. The reaction mixture was poured into water (150 mL). The precipitate was filtered and dried. The crude compound N-methyl-4-[[4-(4-nitropyrazol-1-yl)-5-(trifluoromethyl)pyrimidin-2-yl]amino]benzene sulfonamide was obtained as a white solid.

MS (ES-API positive): 444.0 (M+H)⁺.

### Step 2: 4-[[4-(4-aminopyrazol-1-yl)-5-(trifluoromethyl)pyrimidin-2-yl]amino]-N-methyl-benzenesulfonamide

To a solution of N-methyl-4-[[4-(4-nitropyrazol-1-yl)-5-(trifluoromethyl) pyrimidin-2-yl]amino]benzene sulfonamide (1.9 g, 4.3 mmol) in THF (20 mL) and ethyl acetate (20 mL) was added Pd/C (456.1 mg, 428.5 μmol, 10.0% purity). The mixture was stirred at 25° C. for 4 h under 20 psi H₂. The mixture was filtered and concentrated in vacuo. Compound 4-[[4-(4-aminopyrazol-1-yl)-5-(trifluoromethyl)pyrimidin-2-yl]amino]-N-methyl-benzene sulfonamide was obtained as a white solid.

MS (ES-API positive): 414.0 (M+H)⁺.

### Step 3: N-methyl-4-[[4-(4-pyrrolidin-1-ylpyrazol-1-yl)-5-(trifluoromethyl) pyrimidin-2-yl]amino]benzene sulfonamide

To a solution of 4-[[4-(4-aminopyrazol-1-yl)-5-(trifluoromethyl) pyrimidin-2-yl]amino]-N-methyl-benzene sulfonamide (100.0 mg, 241.9 μmol) and 1,4-dibromobutane (78.4 mg, 362.9 μmol) in DMF (2 mL) was added K₂CO₃ (66.9 mg, 483.8 μmol). The mixture was stirred at 80° C. for 2 h. The reaction mixture was diluted with H₂O (10 mL) and extracted with ethyl acetate (10 mL×3). The combined organic layers were washed with brine, dried over Na₂SO₄, filtered, and concentrated in vacuo. The residue was purified by prep-HPLC (eluting with 50%-70% acetonitrile in water). The compound N-methyl-4-[[4-(4-pyrrolidin-1-ylpyrazol-1-yl)-5-(trifluoromethyl) pyrimidin-2-yl]amino]benzene sulfonamide was obtained as a red solid.

MS (ES-API positive): 468.0 (M+H)⁺.

¹H NMR (400 MHz, Methanol-d₄) δ 8.81 (s, 1H), 8.00-7.95 (m, 3H), 7.83 (d, J=8.8 Hz, 2H), 7.69 (s, 1H), 3.29 (t, J=6.4 Hz, 4H), 2.56 (s, 3H), 2.11-2.04 (m, 4H).

### Example 88: N-methyl-4-[[4-[4-(2-oxopyrrolidin-1-yl) pyrazol-1-yl]-5-(trifluoromethyl) pyrimidin-2-yl]amino]benzene sulfonamide

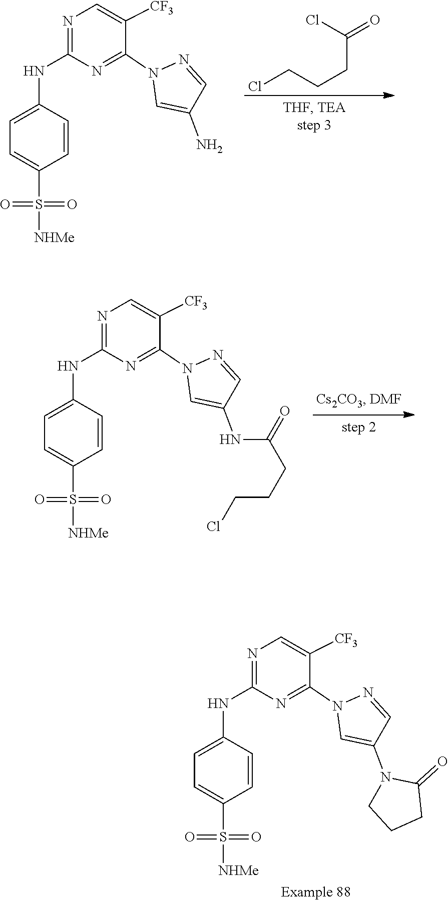 [SMILES: CNS(=O)(=O)c1ccc(Nc2ncc(C(F)(F)F)c(-n3cc(N)cn3)n2)cc1 ; O=C(Cl)CCCCl ; CNS(=O)(=O)c1ccc(Nc2ncc(C(F)(F)F)c(-n3cc(NC(=O)CCCCl)cn3)n2)cc1 ; CNS(=O)(=O)c1ccc(Nc2ncc(C(F)(F)F)c(-n3cc(N4CCCC4=O)cn3)n2)cc1]

### Step 1: 4-chloro-N-[1-[2-[4-(methylsulfamoyl)anilino]-5-(trifluoromethyl)pyrimidin-4-yl]pyrazol-4-yl]butanamide

To a mixture of 4-[[4-(4-aminopyrazol-1-yl)-5-(trifluoromethyl)pyrimidin-2-yl]amino]-N-methyl-benzene sulfonamide (200.0 mg, 483.8 μmol) and 4-chlorobutanoyl chloride (75.0 mg, 532.2 μmol) in THF (4 mL) was added TEA (0.1 mL, 967.6 μmol). The mixture was stirred at 25° C. for 2 h. The reaction mixture was diluted with H₂O (20 mL) and extracted with ethyl acetate (20 mL×3). The combined organic layers were washed with brine, dried over Na₂SO₄, filtered, and concentrated. The residue was purified by flash chromatography (eluting with 0%-40% ethyl acetate in petroleum ether). The compound 4-chloro-N-[1-[2-[4-(methylsulfamoyl)anilino]-5-(trifluoromethyl)pyrimidin-4-yl]pyrazol-4-yl]butanamide was obtained as a yellow oil.

MS (ES-API positive): 518.1 (M+H)⁺.

### Step 2: N-methyl-4-[[4-[4-(2-oxopyrrolidin-1-yl) pyrazol-1-yl]-5-(trifluoromethyl) pyrimidin-2-yl]amino]benzene sulfonamide

To a solution of 4-chloro-N-[1-[2-[4-(methylsulfamoyl) anilino]-5-(trifluoromethyl) pyrimidin-4-yl]pyrazol-4-yl]butanamide (200.0 mg, 386.2 μmol) in DMF (4 mL) was added Cs₂CO₃ (188.7 mg, 579.3 μmol). The mixture was stirred at 60° C. for 2 h. The reaction mixture was diluted with H₂O (20 mL) and extracted with ethyl acetate (20 mL×3). The combined organic layers were washed with brine, dried over Na₂SO₄, filtered, and concentrated. The residue was purified by prep-HPLC (eluting with 43%-63% acetonitrile in water). Compound N-methyl-4-[[4-[4-(2-oxopyrrolidin-1-yl)pyrazol-1-yl]-5-(trifluoromethyl)pyrimidin-2-yl]amino]benzene sulfonamide was obtained as a white solid.

MS (ES-API positive): 482.0 (M+H)⁺.

¹H NMR (400 MHz, Methanol-d₄) δ 10.87 (s, 1H), 8.98 (s, 1H), 8.89 (s, 1H), 8.20 (s, 1H), 8.03-7.94 (m, 2H), 7.76 (d, J=8.7 Hz, 2H), 7.35 (q, J=5.0 Hz, 1H), 3.82 (t, J=7.1 Hz, 2H), 2.49-2.46 (m, 2H), 2.44-2.38 (m, 3H), 2.15 (q, J=7.6 Hz, 2H).

### Example 91 and Example 92: (R)—N-methyl-4-((4-(4-(pyrrolidin-2-yl)-1H-pyrazol-1-yl)-5-(trifluoromethyl)pyrimidin-2-yl)amino)benzenesulfonamide and (S)—N-methyl-4-((4-(4-(pyrrolidin-2-yl)-1H-pyrazol-1-yl)-5-(trifluoromethyl)pyrimidin-2-yl)amino)benzenesulfonamide

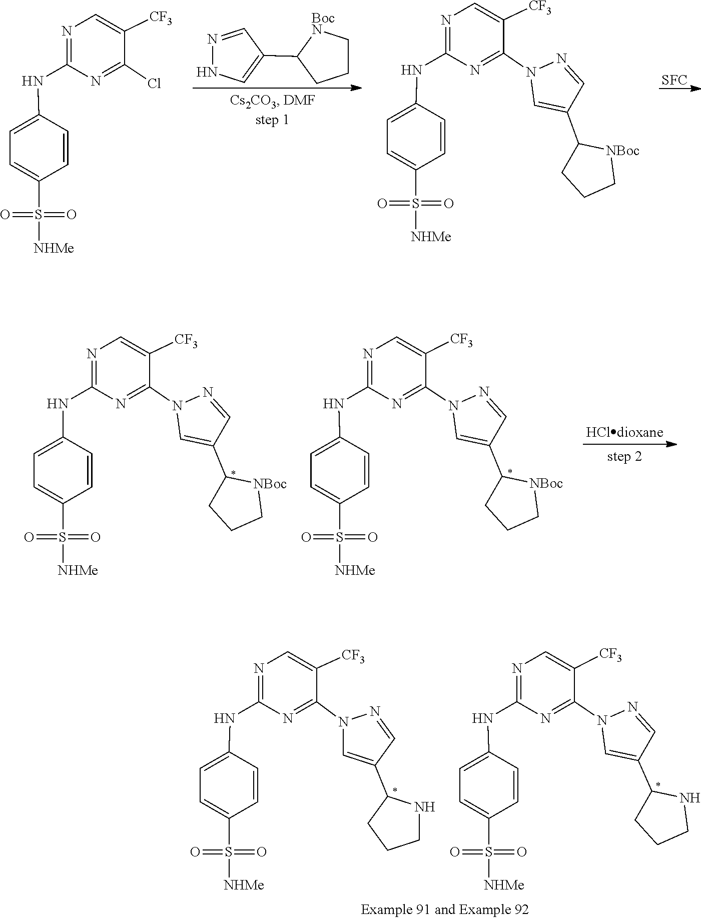

### Step 1: tert-butyl 2-[1-[2-[4-(methylsulfamoyl)anilino]-5-(trifluoromethyl)pyrimidin-4-yl]pyrazol-4-yl]pyrrolidine-1-carboxylate

To a solution of 4-[[4-chloro-5-(trifluoromethyl) pyrimidin-2-yl]amino]-N-methyl-benzene sulfonamide (154.6 mg, 421.4 μmol) in DMSO (1 mL) was added Cs₂CO₃ (274.6 mg, 842.8 μmol) and tert-butyl 2-(1H-pyrazol-4-yl) pyrrolidine-1-carboxylate (100.0 mg, 421.4 μmol). The mixture was stirred at 80° C. for 1 h. The reaction mixture was added to H₂O (20 mL) and extracted with ethyl acetate (20 mL×3). The combined organic layers were washed with brine, dried over Na₂SO₄, filtered, and concentrated. The residue was purified by flash chromatography (eluting with 0%-50% ethyl acetate in petroleum ether). Compound tert-butyl 2-[1-[2-[4-(methylsulfamoyl) anilino]-5-(trifluoromethyl) pyrimidin-4-yl]pyrazol-4-yl]pyrrolidine-1-carboxylate was obtained as a colorless oil. The residue was purified by SFC (column: regis (s,s) whelk-o1 (250 mm×25 mm×10 am); [CO₂-EtOH]; B %: 50%). Compound tert-butyl 2-[1-[2-[4-(methylsulfamoyl)anilino]-5-(trifluoromethyl)pyrimidin-4-yl]pyrazol-4-yl]pyrrolidine-1-carboxylate (Isomer-1) was obtained as colorless oil and tert-butyl 2-[1-[2-[4-(methylsulfamoyl)anilino]-5-(trifluoromethyl)pyrimidin-4-yl]pyrazol-4-yl]pyrrolidine-1-carboxylate (Isomer-2) was obtained as colorless oil. Retention time was 1.527 min and 2.350 min, respectively.

MS (ES-API positive): 568.1 (M+H)⁺.

### Step 2: (R)—N-methyl-4-((4-(4-(pyrrolidin-2-yl)-1H-pyrazol-1-yl)-5-(trifluoromethyl)pyrimidin-2-yl)amino)benzenesulfonamide and (S)—N-methyl-4-((4-(4-(pyrrolidin-2-yl)-1H-pyrazol-1-yl)-5-(trifluoromethyl)pyrimidin-2-yl)amino)benzenesulfonamide

To a solution of tert-butyl 2-[1-[2-[4-(methylsulfamoyl) anilino]-5-(trifluoromethyl) pyrimidin-4-yl]pyrazol-4-yl]pyrrolidine-1-carboxylate (48.0 mg, 84.6 μmol) in dioxane (0.1 mL) was added HCl (4 M in dioxane, 2.0 mL). The mixture was stirred at 25° C. for 1 h. The mixture was concentrated and purified by prep-HPLC (eluting with 22%-42% acetonitrile in water).

Compound N-methyl-4-[[4-(4-pyrrolidin-2-ylpyrazol-1-yl)-5-(trifluoromethyl) pyrimidin-2-yl]amino]benzene sulfonamide was obtained as a white solid.

The other isomer was obtained using the similar conditions.

MS (ES-API positive): 468.1 (M+H)⁺.

Example 91: ¹H NMR (500 MHz, CD₃OD) δ 8.99-8.94 (m, 1H), 8.75-8.72 (m, 1H), 8.05-8.02 (m, 1H), 7.98-7.94 (m, 2H), 7.88-7.83 (m, 2H), 4.81-4.76 (m, 1H), 3.52-3.42 (m, 2H), 2.61-2.52 (m, 4H), 2.39-2.20 (m, 3H).

Example 92: ¹H NMR (400 MHz, Methanol-d₄) δ 8.96 (s, 1H), 8.79-8.70 (m, 1H), 8.03-8.02 (m, 1H), 7.98-7.95 (m, 2H), 7.88-7.83 (m, 2H), 4.82-4.77 (m, 1H), 3.53-3.42 (m, 2H), 2.60-2.53 (m, 4H), 2.37-2.21 (m, 3H).

### Example 93 and Example 94: (R)—N-methyl-4-((4-(4-(pyrrolidin-3-yl)-1H-pyrazol-1-yl)-5-(trifluoromethyl)pyrimidin-2-yl)amino)benzenesulfonamide and (S)—N-methyl-4-((4-(4-(pyrrolidin-3-yl)-1H-pyrazol-1-yl)-5-(trifluoromethyl)pyrimidin-2-yl)amino)benzenesulfonamide

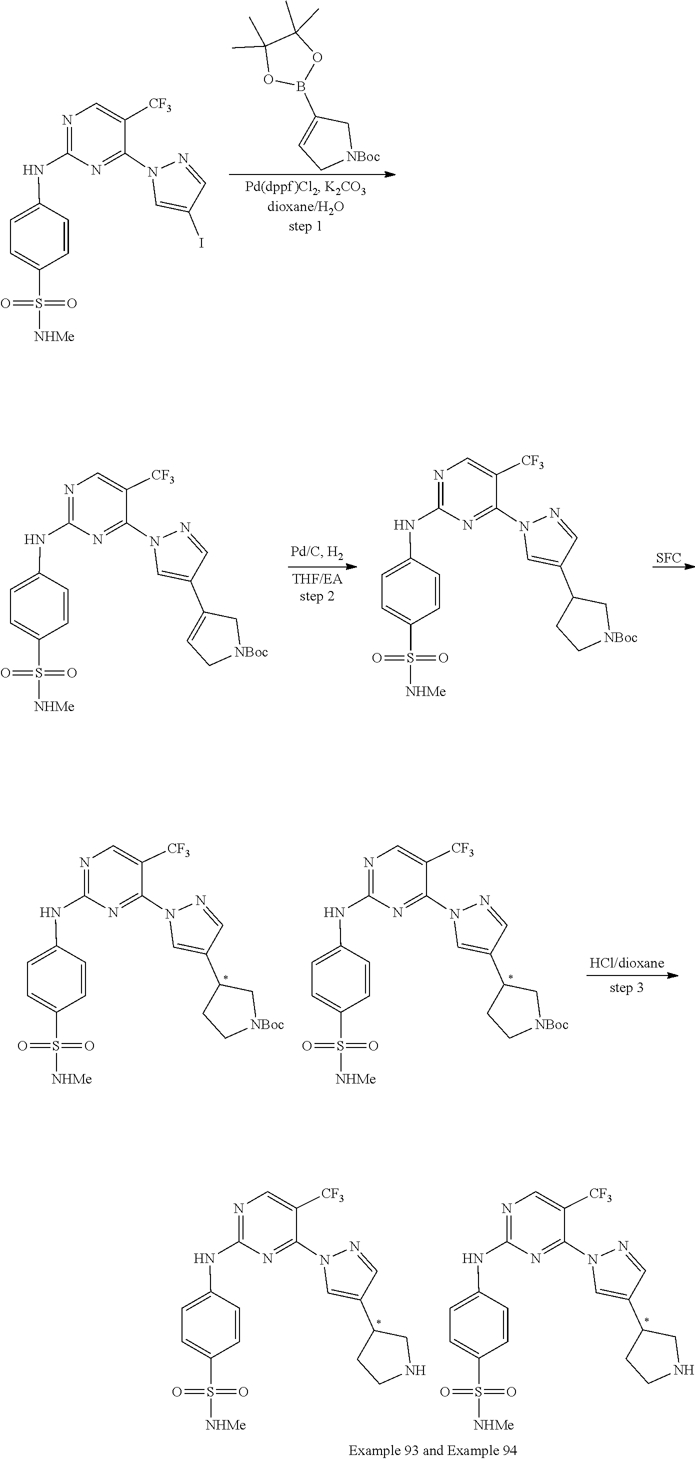

### Step 1: tert-butyl 3-[1-[2-[4-(methylsulfamoyl) anilino]-5-(trifluoromethyl) pyrimidin-4-yl]pyrazol-4-yl]-2,5-dihydropyrrole-1-carboxylate

To a mixture of tert-butyl 3-(4,4,5,5-tetramethyl-1,3,2-dioxaborolan-2-yl)-2,5-dihydropyrrole-1-carboxylate (675.6 mg, 2.3 mmol) and 4-[[4-(4-iodopyrazol-1-yl)-5-(trifluoromethyl)pyrimidin-2-yl]amino]-N-methyl-benzenesulfonamide (1.0 g, 1.9 mmol) in H₂O (5 mL) and dioxane (15 mL) was added Na₂CO₃ (606.5 mg, 5.7 mmol) and Pd(dppf)Cl₂ (139.6 mg, 190.8 μmol). The mixture was stirred at 80° C. for 18 h under N₂ atmosphere. The reaction mixture was poured into water (20 mL) and extracted with ethyl acetate (25 mL×3). The combined organic phases were dried over Na₂SO₄ and concentrated. The residue was purified by flash chromatography (eluting with 0%-45% ethyl acetate in petroleum ether). Compound tert-butyl 3-[1-[2-[4-(methylsulfamoyl) anilino]-5-(trifluoromethyl) pyrimidin-4-yl]pyrazol-4-yl]-2,5-dihydropyrrole-1-carboxylate was obtained as a yellow solid.

MS (ES-API positive): 566.2 (M+H)⁺.

### Step 2: tert-butyl 3-[1-[2-[4-(methylsulfamoyl) anilino]-5-(trifluoromethyl) pyrimidin-4-yl]pyrazol-4-yl]pyrrolidine-1-carboxylate

To a solution of tert-butyl 3-[1-[2-[4-(methylsulfamoyl) anilino]-5-(trifluoromethyl) pyrimidin-4-yl]pyrazol-4-yl]-2,5-dihydropyrrole-1-carboxylate (0.5 g, 937.1 μmol) in THF (5 mL) and ethyl acetate (5 mL) was added Pd/C (99.7 mg, 93.7 μmol, 10.0% purity). The mixture was stirred at 25° C. for 3 h under H₂ (15 psi) atmosphere. The reaction mixture was filtered and concentrated. The residue was further purified by SFC (column: DAICEL CHIRALPAK IG (250 mm×30 mm×10 am); [CO₂-MeOH (0.1% NH₃—H₂O)]; B %: 50%). Compound tert-butyl 3-[1-[2-[4-(methylsulfamoyl) anilino]-5-(trifluoromethyl) pyrimidin-4-yl]pyrazol-4-yl]pyrrolidine-1-carboxylate (Isomer-1) was obtained as a white solid. Compound tert-butyl 3-[1-[2-[4-(methylsulfamoyl) anilino]-5-(trifluoromethyl) pyrimidin-4-yl]pyrazol-4-yl]pyrrolidine-1-carboxylate (Isomer-2) was obtained as a white solid. Retention time was 3.168 min and 3.851 min, respectively.

MS (ES-API positive): 568.2 (M+H)⁺.

### Step 3: (R)—N-methyl-4-((4-(4-(pyrrolidin-3-yl)-1H-pyrazol-1-yl)-5-(trifluoromethyl)pyrimidin-2-yl)amino)benzenesulfonamide and (S)—N-methyl-4-((4-(4-(pyrrolidin-3-yl)-1H-pyrazol-1-yl)-5-(trifluoromethyl)pyrimidin-2-yl)amino)benzenesulfonamide

A solution of tert-butyl 3-[1-[2-[4-(methylsulfamoyl) anilino]-5-(trifluoromethyl) pyrimidin-4-yl]pyrazol-4-yl]pyrrolidine-1-carboxylate (40.0 mg, 70.5 μmol) in dioxane was added HCl (4 M in dioxane, 2 mL). The mixture was stirred at 20° C. for 2 h. The reaction mixture was filtered and concentrated. The residue was purified by prep-HPLC (eluting with 25%-45% acetonitrile in water). Compound N-methyl-4-[[4-(4-pyrrolidin-3-ylpyrazol-1-yl)-5-(trifluoromethyl) pyrimidin-2-yl]amino]benzene sulfonamide was obtained as a white solid. The other isomer was obtained using similar conditions.

MS (ES-API positive): 468.1 (M+H)⁺.

Example 93: ¹H NMR (400 MHz, CD₃OD) δ 8.92 (s, 1H), 8.55 (s, 1H), 7.97 (d, J=8.8 Hz, 2H), 7.91 (s, 1H), 7.85 (d, J=8.8 Hz, 2H), 3.76 (dd, J=7.9, 11.2 Hz, 1H), 3.70-3.53 (m, 2H), 3.48-3.39 (m, 1H), 3.25 (dd, J=9.5, 11.1 Hz, 1H), 2.56 (s, 4H), 2.23-2.11 (m, 1H).

Example 94: ¹H NMR (400 MHz, CD₃OD) δ 8.96-8.83 (m, 1H), 8.55 (s, 1H), 7.97 (d, J=8.8 Hz, 2H), 7.91 (s, 1H), 7.85 (d, J=8.8 Hz, 2H), 3.83-3.71 (m, 1H), 3.68-3.53 (m, 2H), 3.47-3.40 (m, 1H), 3.25 (dd, J=9.7, 11.0 Hz, 1H), 2.56 (s, 4H), 2.25-2.06 (m, 1H).

### Example 103: 4-[[4-[4-(3-hydroxycyclobutyl) pyrazol-1-yl]-5-(trifluoromethyl) pyrimidin-2-yl]amino]-N-methyl-benzene sulfonamide

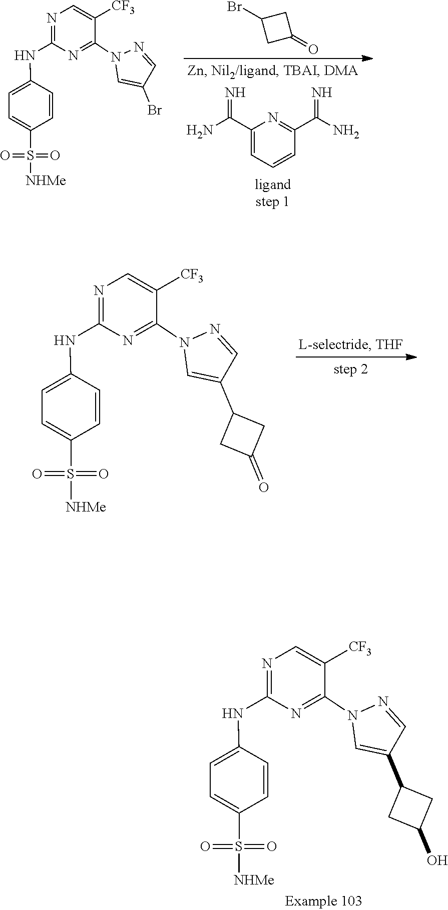

### Step 1: N-methyl-4-[[4-[4-(3-oxocyclobutyl) pyrazol-1-yl]-5-(trifluoromethyl) pyrimidin-2-yl]amino]benzene sulfonamide

To a mixture of 4-[[4-(4-bromopyrazol-1-yl)-5-(trifluoromethyl)pyrimidin-2-yl]amino]-N-methyl-benzenesulfonamide (0.4 g, 838.1 μmol) and 3-bromocyclobutanone (187.3 mg, 1.3 mmol) in DMA (10 mL) was added NiI₂ (52.4 mg, 167.6 μmol), TBAI (464.4 mg, 1.3 mmol), Manganese (184.2 mg, 3.4 mmol) and pyridine-2,6-dicarboxamidine (27.4 mg, 167.6 μmol). The mixture was stirred at 60° C. for 4 h under N₂. The reaction mixture was poured into H₂O (30 mL) and extracted with EA (15 mL×3). The organic phase was washed with brine, dried over Na₂SO₄, filtered, and concentrated. The residue was purified by flash chromatography (eluting with 0%-50% ethyl acetate in petroleum ether). Compound N-methyl-4-[[4-[4-(3-oxocyclobutyl) pyrazol-1-yl]-5-(trifluoromethyl) pyrimidin-2-yl]amino]benzene sulfonamide was obtained as yellow oil.

MS (ES-API positive): 467.2 (M+H)⁺.

### Step 2: 4-[[4-[4-(3-hydroxycyclobutyl) pyrazol-1-yl]-5-(trifluoromethyl) pyrimidin-2-yl]amino]-N-methyl-benzene sulfonamide

To a solution of N-methyl-4-[[4-[4-(3-oxocyclobutyl) pyrazol-1-yl]-5-(trifluoromethyl) pyrimidin-2-yl]amino]benzene sulfonamide (80.0 mg, 171.5 μmol) in THF (2 mL) was added L-Selectride (343.0 μmol, 75.0 μL) at −78° C. and stirred for 1 h under N₂. The reaction mixture was quenched with H₂O (4 mL) at −60° C., then warmed to 20° C. and stirred for 10 min. The mixture was extracted with ethyl acetate (5 mL×3), washed with brine, dried over Na₂SO₄, filtered, and concentrated. The residue was purified by prep-HPLC (eluting with 35%-65% acetonitrile in water). Compound 4-[[4-[4-(3-hydroxycyclobutyl) pyrazol-1-yl]-5-(trifluoromethyl) pyrimidin-2-yl]amino]-N-methyl-benzene sulfonamide was obtained as a white solid.

MS (ES-API positive): 469.0 (M+H)⁺.

¹H NMR (400 MHz, Methanol-d₄) δ 8.87 (s, 1H), 8.36 (s, 1H), 7.96 (d, J=8.7 Hz, 2H), 7.82 (d, J=8.8 Hz, 2H), 7.77 (s, 1H), 4.19 (q, J=7.5 Hz, 1H), 3.02-2.90 (m, 1H), 2.80-2.69 (m, 2H), 2.54 (s, 3H), 2.05-1.94 (m, 2H).

### Example 104 and Example 105: 4-((4-(4-((1s,3s)-3-hydroxy-3-methylcyclobutyl)-1H-pyrazol-1-yl)-5-(trifluoromethyl)pyrimidin-2-yl)amino)-N-methylbenzenesulfonamide and 4-((4-(4-((1r,3r)-3-hydroxy-3-methylcyclobutyl)-1H-pyrazol-1-yl)-5-(trifluoromethyl)pyrimidin-2-yl)amino)-N-methylbenzenesulfonamide

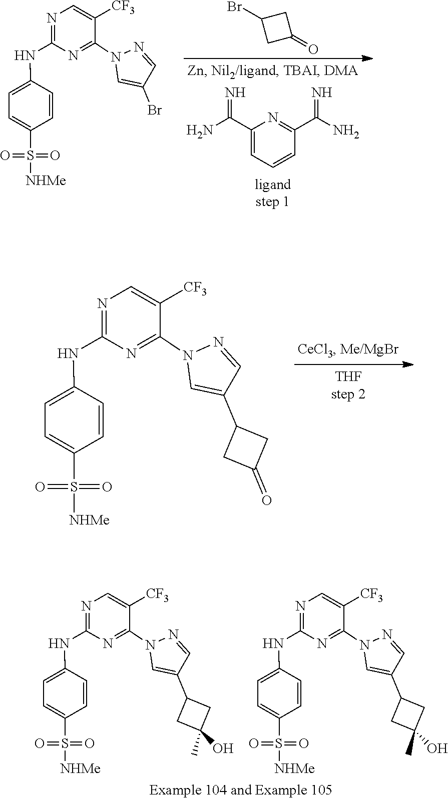

### Step 1: N-methyl-4-[[4-[4-(3-oxocyclobutyl) pyrazol-1-yl]-5-(trifluoromethyl) pyrimidin-2-yl]amino]benzene sulfonamide

Same as the previous example.

### Step 2: 4-[[4-[4-(3-hydroxy-3-methyl-cyclobutyl)pyrazol-1-yl]-5-(trifluoromethyl)pyrimidin-2-yl]amino]-N-methyl-benzenesulfonamide

To a mixture of N-methyl-4-[[4-[4-(3-oxocyclobutyl) pyrazol-1-yl]-5-(trifluoromethyl) pyrimidin-2-yl]amino]benzene sulfonamide (60.0 mg, 128.6 μmol) and CeCl₃ (6.3 mg, 25.7 μmol) in THF (2 mL) was added MeMgBr (3 M, 257.3 μL) at 0° C. The mixture was stirred at 0° C. for 2 h under N₂. The reaction mixture was quenched with sat. NH₄Cl (6 mL) at 0° C. and extracted with ethyl acetate (5 mL×3). The organic phases were washed with brine, dried over Na₂SO₄, filtered, and concentrated. The residue was purified by prep-HPLC (eluting with 50%-70% acetonitrile in water), followed by prep-HPLC (eluting with 40%-60% acetonitrile in water).

4-((4-(4-((1s,3s)-3-hydroxy-3-methylcyclobutyl)-1H-pyrazol-1-yl)-5-(trifluoromethyl)pyrimidin-2-yl)amino)-N-methylbenzenesulfonamide and 4-((4-(4-((1r,3r)-3-hydroxy-3-methylcyclobutyl)-1H-pyrazol-1-yl)-5-(trifluoromethyl)pyrimidin-2-yl)amino)-N-methylbenzenesulfonamide were obtained as a white solid.

MS (ES-API positive): 483.1 (M+H)⁺.

Example 104: ¹H NMR (400 MHz, Methanol-d₄) δ 8.86 (s, 1H), 8.37 (s, 1H), 7.96 (d, J=8.9 Hz, 2H), 7.87-7.79 (m, 2H), 7.75 (s, 1H), 3.58 (q, J=8.2 Hz, 1H), 2.58-2.50 (m, 5H), 2.22-2.13 (m, 2H), 1.35 (s, 3H).

Example 105: ¹H NMR (400 MHz, Methanol-d₄) δ 8.86 (s, 1H), 8.36 (s, 1H), 7.96 (d, J=8.9 Hz, 2H), 7.87-7.81 (m, 2H), 7.79 (s, 1H), 3.06 (q, J=8.9 Hz, 1H), 2.54 (s, 3H), 2.51-2.45 (m, 2H), 2.23-2.14 (m, 2H), 1.44 (s, 3H).

### Example 106: 4-((4-(4-((1r,3r)-3-hydroxycyclobutyl)-1H-pyrazol-1-yl)-5-(trifluoromethyl)pyrimidin-2-yl)amino)-N-methylbenzenesulfonamide

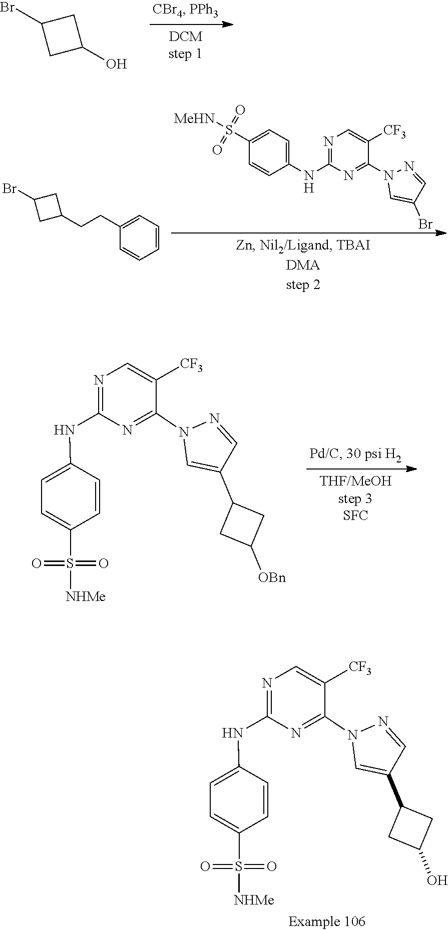

### Step 1: (3-bromocyclobutoxy) methylbenzene

To a solution of 3-benzyloxycyclobutanol (2.0 g, 11.2 mmol) in DCM (20 mL) was added CBr₄ (5.6 g, 16.8 mmol) and PPh₃ (4.4 g, 16.8 mmol). The mixture was stirred at 25° C. for 2 h. The reaction mixture was poured into water (20 mL) and extracted with ethyl acetate (30 mL×3). The combined organic layers were washed with brine, dried over Na₂SO₄, filtered, and concentrated. The residue was purified by flash chromatography (eluting with 0%-5% ethyl acetate in petroleum ether). Compound (3-bromocyclobutoxy) methylbenzene was obtained as a colorless oil.

MS (ES-API positive): 241.1 (M+H)⁺.

### Step 2: 4-[[4-[4-(3-benzyloxycyclobutyl) pyrazol-1-yl]-5-(trifluoromethyl) pyrimidin-2-yl]amino]-N-methyl-benzene sulfonamide

To a solution of pyridine-2,6-dicarboxamidine (44.5 mg, 272.4 μmol), (3-bromocyclobutoxy)methylbenzene (656.8 mg, 2.7 mmol) and 4-[[4-(4-bromopyrazol-1-yl)-5-(trifluoromethyl)pyrimidin-2-yl]amino]-N-methyl-benzenesulfonamide (650.0 mg, 1.4 mmol) in DMA (7 mL) was added TBAI (628.8 mg, 1.7 mmol), NiI₂ (59.8 mg, 272.4 μmol), Manganese (299.3 mg, 5.5 mmol) and TFA (1.4 mmol, 101.2 μL). The mixture was stirred at 80° C. for 4 h and filtered. The filtrate was poured into water (15 mL) and extracted with ethyl acetate (15 mL×3), dried over Na₂SO₄, and concentrated. The crude product was purified by pre-HPLC (eluting with 0%-30% acetonitrile in water). Compound 4-[[4-[4-(3-benzyloxycyclobutyl) pyrazol-1-yl]-5-(trifluoromethyl) pyrimidin-2-yl]amino]-N-methyl-benzene sulfonamide was obtained as a yellow oil.

MS (ES-API positive): 559.3 (M+H)⁺.

### Step 3: 4-((4-(4-((1r,3r)-3-hydroxycyclobutyl)-1H-pyrazol-1-yl)-5-(trifluoromethyl)pyrimidin-2-yl)amino)-N-methylbenzenesulfonamide

To a solution of 4-[[4-[4-(3-benzyloxycyclobutyl) pyrazol-1-yl]-5-(trifluoromethyl) pyrimidin-2-yl]amino]-N-methyl-benzene sulfonamide (300.0 mg, 537.1 μmol) in THF (5 mL) and MeOH (5 mL) was added Pd/C (57.2 mg, 53.7 μmol, 10.0% purity). The mixture was stirred at 25° C. for 5 h under 30 psi H₂. The reaction mixture was filtered and concentrated. The residue was purified by prep-HPLC (eluting with 44%-64% acetonitrile in water) to give desired compound, which was further separated by SFC (column: DAICEL CHIRALPAK IC (250 mm×30 mm×10 am); [CO₂-i-PrOH (0.1% NH₃H₂O)]; B %: 50%) to get the compound 4-((4-(4-((1r,3r)-3-hydroxycyclobutyl)-1H-pyrazol-1-yl)-5-(trifluoromethyl)pyrimidin-2-yl)amino)-N-methylbenzenesulfonamide as a white solid (Retention time: 1.562 min).

MS (ES-API positive): 469.1 (M+H)⁺.

¹H NMR (400 MHz, Methanol-d₄) δ 8.88 (s, 1H), 8.42 (s, 1H), 7.98 (d, J=8.8 Hz, 2H), 7.88-7.72 (m, 3H), 4.62-4.42 (m, 1H), 3.55-3.41 (m, 1H), 2.56 (s, 3H), 2.48-2.37 (m, 4H).

### Example 116: 4-((5-(difluoromethyl)-4-(1H-pyrazol-1-yl)pyrimidin-2-yl)amino)-N-(2-hydroxyethyl)benzenesulfonamide

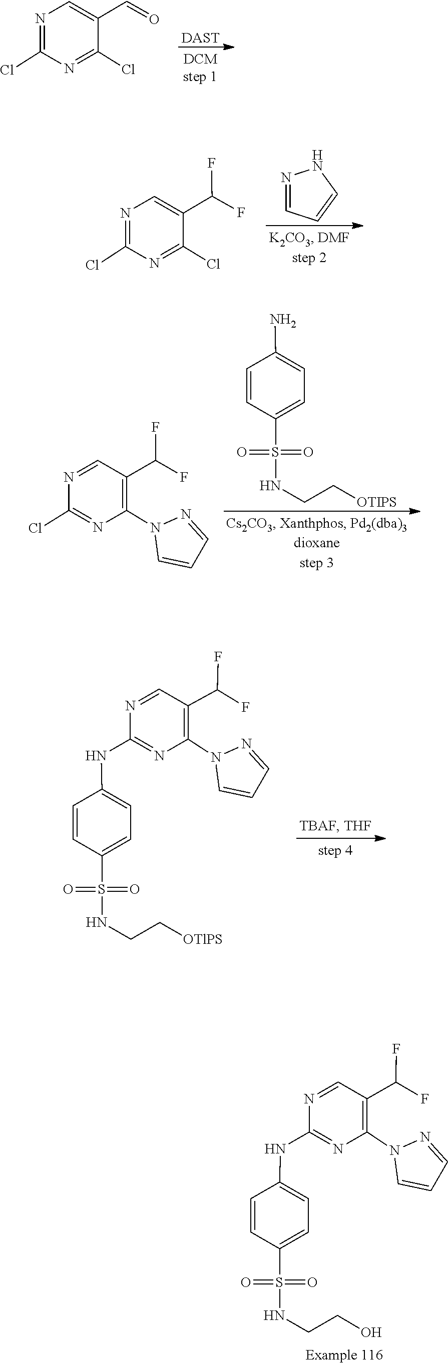

### Example 116

### Step 1: 2,4-dichloro-5-(difluoromethyl)pyrimidine

To a solution of 2,4-dichloropyrimidine-5-carbaldehyde (1.0 g, 5.7 mmol) in DCM (10 mL) was added DAST (1.5 mL, 11.4 mmol) under 0° C. The reaction mixture was stirred at rt for 2 h and concentrated. The residue was purified by flash chromatography (eluting with 10% ethyl acetate in petroleum ether) to afford 2,4-dichloro-5-(difluoromethyl)pyrimidine as a white solid.

¹H NMR (400 MHz, Chloroform-d) δ 8.81 (s, 1H), 6.89 (t, J=53.7 Hz, 1H).

### Step 2: 2-chloro-5-(difluoromethyl)-4-(pyrazol-1-yl)pyrimidine

To a solution of 2,4-dichloro-5-(difluoromethyl)pyrimidine (445.0 mg, 2.2 mmol) in DMF (6 mL) was added 1H-pyrazole (152.2 mg, 2.2 mmol) and K₂CO₃ (309.1 mg, 2.2 mmol) and stirred at rt for 2 h. The reaction mixture was diluted with water, extracted with ethyl acetate, and concentrated. The residue was purified by flash chromatography (eluting with 10%-20% ethyl acetate in petroleum ether) to afford 2-chloro-5-(difluoromethyl)-4-(pyrazol-1-yl)pyrimidine as a white solid.

MS (ES-API positive): 231.1 (M+H)⁺.

### Step 3: 4-{[5-(difluoro methyl)-4-(pyrazol-1-yl) pyrimidin-2-yl]amino}-N-[5-methyl-4,4-di(prop-2-yl)-3-oxa-4-silahex-1-yl]benzene sulfonamide

To a solution of 2-chloro-5-(difluoro methyl)-4-(pyrazol-1-yl)pyrimidine (150.0 mg, 0.7 mmol) in dioxane (6.0 mL) was added 4-amino-N-[5-methyl-4,4-di(prop-2-yl)-3-oxa-4-silahex-1-yl]benzenesulfonamide (290.8 mg, 0.8 mmol), Pd₂(dba)₃ (178.7 mg, 0.2 mmol), Xantphos (150.5 mg, 0.3 mmol) and Cs₂CO₃ (275.5 mg, 0.8 mmol). The reaction mixture was stirred at 90° C. under N₂ for 1 h. The mixture was extracted with ethyl acetate and concentrated. The residue was purified by flash chromatography (eluting with 30% ethyl acetate in petroleum ether) to afford 4-{[5-(difluoro methyl)-4-(pyrazol-1-yl) pyrimidin-2-yl]amino}-N-[5-methyl-4,4-di(prop-2-yl)-3-oxa-4-silahex-1-yl]benzene sulfonamide as a yellow solid.

MS (ES-API positive): 567.2 (M+H)⁺.

### Step 4: 4-{[5-(difluoro methyl)-4-(pyrazol-1-yl) pyrimidin-2-yl]amino}-N-(2-hydroxyethyl) benzene sulfonamide

To a solution of 4-{[5-(difluoro methyl)-4-(pyrazol-1-yl) pyrimidin-2-yl]amino}-N-[5-methyl-4,4-di(prop-2-yl)-3-oxa-4-silahex-1-yl]benzene sulfonamide (226.0 mg, 0.4 mmol) in THF (5.0 mL) was added TBAF (4 M in THF, 0.3 mL). The reaction was stirred at rt for 0.5 h. The reaction mixture was concentrated in vacuo. The residue was diluted with H₂O, extracted with ethyl acetate, and concentrated. The residue was purified by flash chromatography (eluting with 9% methanol in dichloromethane) to afford 4-{[5-(difluoro methyl)-4-(pyrazol-1-yl) pyrimidin-2-yl]amino}-N-(2-hydroxyethyl) benzene sulfonamide as a white solid.

MS (ES-API positive): 411.0 (M+H)⁺.

¹H NMR (400 MHz, DMSO-d₆) δ 10.66 (s, 1H), 8.91 (s, 1H), 8.71 (d, J=2.7 Hz, 1H), 8.03 (d, J=1.6 Hz, 1H), 7.95 (d, J=8.9 Hz, 2H), 7.80 (dd, J=9.3, 7.3 Hz, 3H), 7.46 (t, J=5.9 Hz, 1H), 6.72 (dd, J=2.8, 1.6 Hz, 1H), 4.70 (t, J=5.5 Hz, 1H), 3.43-3.37 (m, 2H), 2.79 (q, J=6.2 Hz, 2H).

### Example 130: 3-[4-[[4-pyrazol-1-yl-5-(trifluoromethyl)pyrimidin-2-yl]amino]phenyl]sulfonylpropan-1-ol

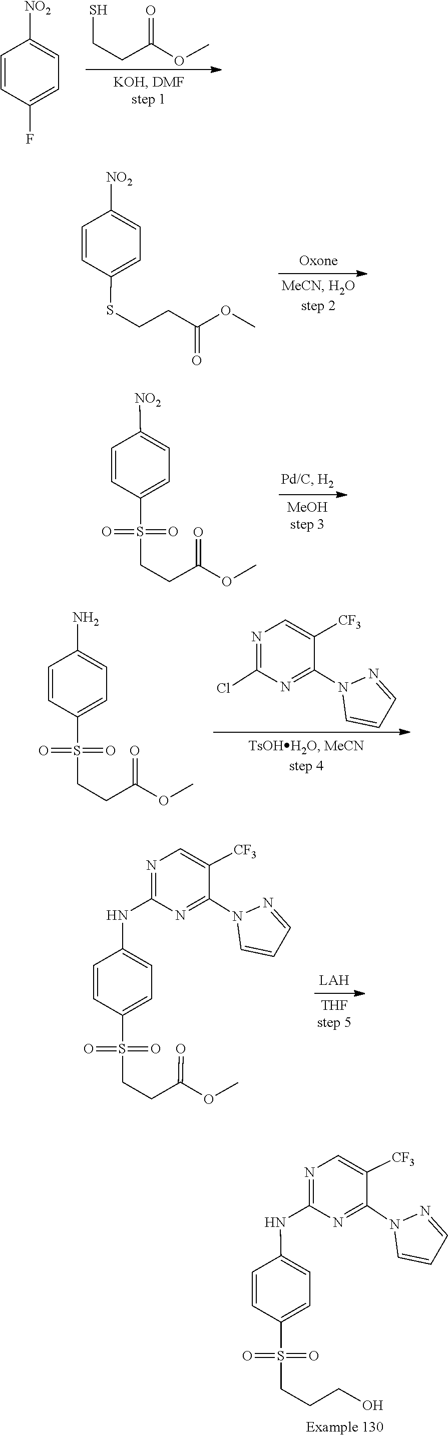

### Step 1: methyl 3-(4-nitrophenyl)sulfanylpropanoate

To a solution of 1-fluoro-4-nitrobenzene (3.4 g, 24.0 mmol) and methyl 3-sulfanylpropanoate (4.9 g, 40.8 mmol) in DMF (50 mL) was added KOH (1.9 g, 33.6 mmol) at 0° C. The mixture was stirred at 80° C. for 1 h under N₂. The reaction mixture was quenched with ice-water (120 mL), filtered and recrystallized (methanol and water). Compound methyl 3-(4-nitrophenyl)sulfanylpropanoate as a pale yellow solid, which was used directly for the next step without further purification.

### Step 2: methyl 3-(4-nitrophenyl)sulfonylpropanoate

A solution of Oxone (22.9 g, 37.3 mmol) in H₂O (600 mL) was added into a solution of methyl 3-(4-nitrophenyl)sulfanylpropanoate (3.0 g, 12.4 mmol) in acetonitrile (300 mL) at 0° C. The mixture was stirred at rt for 3 h. The reaction mixture was quenched with saturated Na₂SO₃ aq. (100 mL) at 0° C., and extracted with ethyl acetate (100 mL×3). The combined organic layers were washed with brine, dried over Na₂SO₄, filtered, and concentrated. The residue was obtained as a pale yellow solid, which was used directly for next step without further purification.

MS (ES-API positive): 296.2 (M+Na)⁺.

### Step 3: methyl 3-(4-aminophenyl)sulfonylpropanoate

To a solution of methyl 3-(4-nitrophenyl)sulfonylpropanoate (2.0 g, 7.3 mmol) in MeOH (100 mL) was added Pd/C (779.0 mg, 0.7 mmol, 10.0% purity) and stirred at rt for 16 h under H₂ (40 psi). The reaction mixture was filtered and concentrated to afford the desired compound as a pale yellow solid, which was used directly for next step without further purification.

MS (ES-API positive): 261.2 (M+H)⁺.

### Step 4: methyl 3-[4-[[4-pyrazol-1-yl-5-(trifluoromethyl)pyrimidin-2-yl]amino]phenyl]sulfonylpropanoate

To a solution of methyl 3-(4-aminophenyl)sulfonylpropanoate (1.0 g, 2.1 mmol) in ACN (16 mL) was added TsOH·H₂O (784.0 mg, 2.1 mmol) and 2-chloro-4-pyrazol-1-yl-5-(trifluoromethyl) pyrimidine (1.0 g, 2.1 mmol). The mixture was stirred at 80° C. for 2 h. The reaction mixture was concentrated and diluted with NaHCO₃ (20 mL). The combined organic layers were washed with brine, dried over Na₂SO₄, filtered, and concentrated. The residue was purified by flash chromatography (eluting with 0%-33% ethyl acetate in petroleum ether). The desired compound was obtained as a yellow solid.

MS (ES-API positive): 456.0 (M+H)⁺.

### Step 5: 3-[4-[[4-pyrazol-1-yl-5-(trifluoromethyl)pyrimidin-2-yl]amino]phenyl]sulfonylpropan-1-ol

To a mixture of methyl 3-[4-[[4-pyrazol-1-yl-5-(trifluoromethyl)pyrimidin-2-yl]amino]phenyl]sulfonyl propanoate (600.0 mg, 1.3 mmol) in THF (5 mL) was added LAH (2.5 M, 0.5 mL) at −78° C. and stirred at −40° C. for 1 h. The reaction mixture was quenched with Na₂SO₄-10H₂O (0.6 g) at 0° C., filtered and concentrated. The residue was purified with by prep-HPLC (eluting with 35%-53% acetonitrile in water) to give 3-[4-[[4-pyrazol-1-yl-5-(trifluoromethyl)pyrimidin-2-yl]amino]phenyl]sulfonylpropan-1-ol as a white solid.

MS (ES-API positive): 428.3 (M+H)⁺.

¹H NMR (400 MHz, DMSO-d₆) δ 10.94 (s, 1H), 9.01 (s, 1H), 8.63 (d, J=2.6 Hz, 1H), 8.07-7.94 (m, 3H), 7.88 (d, J=8.8 Hz, 2H), 6.70 (d, J=2.5 Hz, 1H), 3.41 (t, J=6.2 Hz, 2H), 3.33-3.14 (m, 2H), 1.76-1.61 (m, 2H).

### Example 136: N-(2-hydroxyethyl)-5-[[4-pyrazol-1-yl-5-(trifluoromethyl)pyrimidin-2-yl]amino]pyridine-2-sulfonamide

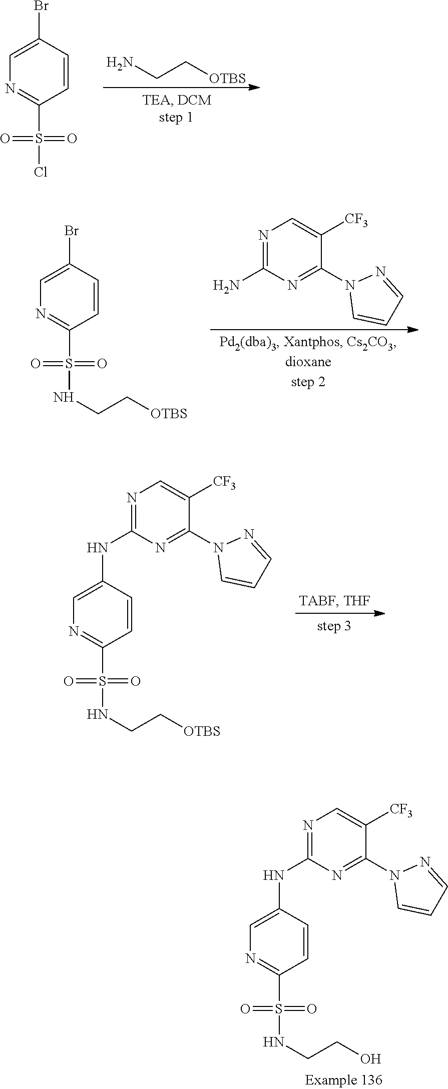 [SMILES: O=S(=O)(Cl)c1ccc(Br)cn1 ; CC(C)(C)[Si](C)(C)OCCN ; CC(C)(C)[Si](C)(C)OCCNS(=O)(=O)c1ccc(Br)cn1 ; Nc1ncc(C(F)(F)F)c(-n2cccn2)n1 ; CC(C)(C)[Si](C)(C)OCCNS(=O)(=O)c1ccc(Nc2ncc(C(F)(F)F)c(-n3cccn3)n2)cn1 ; O=S(=O)(NCCO)c1ccc(Nc2ncc(C(F)(F)F)c(-n3cccn3)n2)cn1]

### Step 1: 5-bromo-N-[2-[tert-butyl(dimethyl)silyl]oxyethyl]pyridine-2-sulfonamide

To a solution of 5-bromopyridine-2-sulfonyl chloride (1.0 g, 3.9 mmol) and 2-[tert-butyl(dimethyl)silyl]oxyethanamine (820.0 mg, 4.7 mmol) in DCM (15 mL) was added TEA (7.8 mmol, 1.0 mL). The reaction was stirred at rt for 2 h. The reaction was extracted with DCM (15 mL×2), dried over Na₂SO₄, filtered, and concentrated. The residue was purified by flash chromatography (eluting with 0%-50% ethyl acetate in petroleum ether). Compound 5-bromo-N-[2-[tert-butyl(dimethyl)silyl]oxyethyl]pyridine-2-sulfonamide was obtained as a white solid.

MS (ES-API positive): 396.8, 394.8 (M+H)⁺.

### Step 2: N-[2-[tert-butyl(dimethyl)silyl]oxyethyl]-5-[[4-pyrazol-1-yl-5-(trifluoromethyl) pyrimidin-2-yl]amino]pyridine-2-sulfonamide

A mixture of 5-bromo-N-[2-[tert-butyl(dimethyl)silyl]oxyethyl]pyridine-2-sulfonamide (400.0 mg, 1.0 mmol), 4-pyrazol-1-yl-5-(trifluoromethyl)pyrimidin-2-amine (232.0 mg, 1.0 mmol), Xantphos (120.0 mg, 0.2 mmol), Pd₂(dba)₃ (93.0 mg, 0.1 mmol) and Cs₂CO₃ (667.0 mg, 2.0 mmol) in dioxane (4 mL) was degassed and purged with N₂ for 3 times. The mixture was stirred at 100° C. for 16 h. The reaction mixture was quenched with water (10 mL) and extracted with ethyl acetate (10 mL×3). The combined organic layers were washed with brine, dried over Na₂SO₄, filtered, and concentrated. The residue was purified by flash chromatography (eluting with 0%-50% ethyl acetate in petroleum ether). Compound N-[2-[tert-butyl(dimethyl)silyl]oxyethyl]-5-[[4-pyrazol-1-yl-5-(trifluoromethyl) pyrimidin-2-yl]amino]pyridine-2-sulfonamide was obtained as a white solid.

MS (ES-API positive): 544.1 (M+H)⁺.

### Step 3: N-(2-hydroxyethyl)-5-[[4-pyrazol-1-yl-5-(trifluoromethyl) pyrimidin-2-yl]amino]pyridine-2-sulfonamide

N-[2-[tert-butyl(dimethyl)silyl]oxyethyl]-5-[[4-pyrazol-1-yl-5-(trifluoromethyl) pyrimidin-2-yl]amino]pyridine-2-sulfonamide (100.0 mg, 184.0 μmol) in THF (1 mL) was added TBAF (1 M, 221.0 μL) at 0° C. and stirred at this temperature for 1 h. The reaction mixture was quenched with water (5 mL) and extracted with ethyl acetate (10 mL×3). The combined organic layers were washed with brine (10 mL), dried over Na₂SO₄, filtered, and concentrated. The residue was triturated with ethyl acetate (5 mL) at rt for 10 min and filtered. The filter cake was purified by prep-HPLC (eluting with 31%-51% acetonitrile in water) to give N-(2-hydroxyethyl)-5-[[4-pyrazol-1-yl-5-(trifluoromethyl)pyrimidin-2-yl]amino]pyridine-2-sulfonamide as a white solid.

MS (ES-API positive): 430.1 (M+H)⁺.

¹H NMR (500 MHz, DMSO-d₆) δ 11.01 (s, 1H), 9.12-8.91 (m, 2H), 8.60 (d, J=2.6 Hz, 1H), 8.43 (d, J=2.4 Hz, 1H), 8.06-7.89 (m, 2H), 7.69 (s, 1H), 6.71 (d, J=2.6 Hz, 1H), 4.66 (t, J=5.6 Hz, 1H), 3.37 (q, J=6.4 Hz, 2H), 2.98-2.92 (m, 2H).

### Example 138: ((4-((4-(1H-pyrazol-1-yl)-5-(trifluoromethyl)pyrimidin-2-yl)amino)phenyl)sulfonyl)glycine

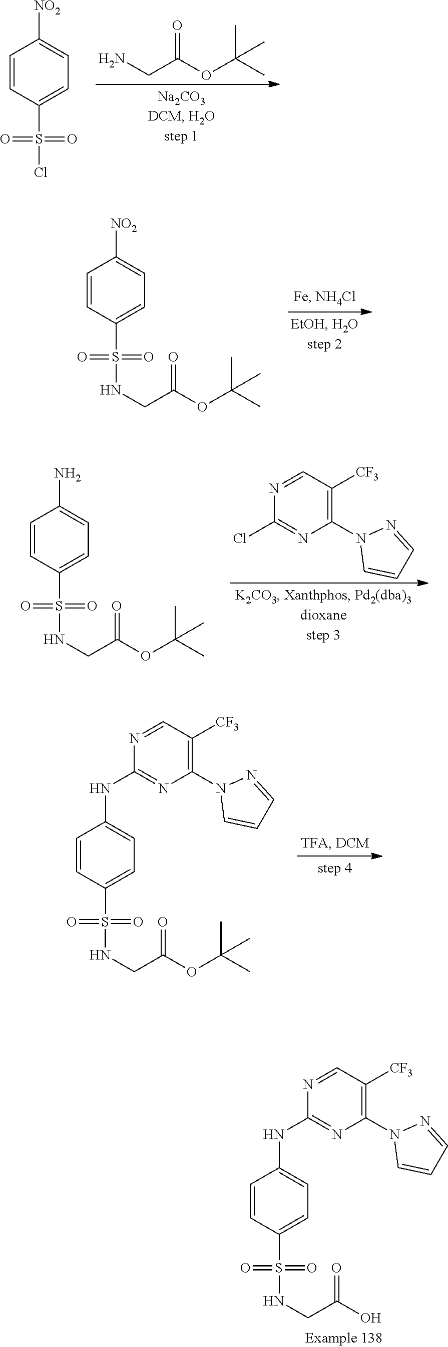

### Example 138

### Step 1: tert-butyl ((4-nitrophenyl)sulfonyl)glycinate

To a mixture of 2-methylpropan-2-yl amino acetate (355.2 mg, 2.7 mmol) in DCM (6 mL) and H₂O (6 mL) was added Na₂CO₃ (358.7 mg, 3.4 mmol) and 4-nitrobenzenesulfonyl chloride (500.0 mg, 2.3 mmol) at 0° C. The reaction mixture was stirred at rt for 1.5 h, extracted with DCM and concentrated to afford a residue which was used for next step without further purification.

### Step 2: tert-butyl ((4-aminophenyl)sulfonyl)glycinate

To a mixture of tert-butyl ((4-nitrophenyl)sulfonyl)glycinate (713.7 mg, 2.3 mmol) and NH₄Cl (1.2 g, 22.6 mmol) in EtOH (9 mL) and H₂O (6 mL) was added Fe (503.9 mg, 9.0 mmol). The reaction mixture was stirred at 65° C. for 8 h. The mixture was diluted with ethyl acetate (30 mL) and filtered. The filtrate was extracted with ethyl acetate and concentrated. The residue was purified by flash chromatography (eluting with 66% ethyl acetate in petroleum ether) to afford tert-butyl ((4-aminophenyl)sulfonyl)glycinate as a yellow solid.

MS (ES-API negative): 285.1 (M+H)⁺.

### Step 3: tert-butyl ((4-((4-(1H-pyrazol-1-yl)-5-(trifluoromethyl)pyrimidin-2-yl)amino)phenyl)sulfonyl)glycinate

To a mixture of 2-chloro-4-(pyrazol-1-yl)-5-(trifluoromethyl) pyrimidine (100.0 mg, 0.4 mmol) in dioxane (2 mL) was added tert-butyl ((4-aminophenyl)sulfonyl)glycinate (115.1 mg, 0.4 mmol), Pd₂(dba)₃ (110.5 mg, 0.1 mmol), Xantphos (93.1 mg, 0.2 mmol) and K₂CO₃ (61.1 mg, 0.4 mmol). The reaction mixture was stirred at 85° C. under N₂ for 1.5 h. The mixture was filtered, extracted with ethyl acetate and concentrated. The residue was purified by flash chromatography (eluting with 50%-66% ethyl acetate in petroleum ether) to afford tert-butyl ((4-((4-(1H-pyrazol-1-yl)-5-(trifluoromethyl)pyrimidin-2-yl)amino)phenyl)sulfonyl)glycinate as a yellow solid.

MS (ES-API positive): 499.2 (M+H)⁺.

### Step 4: ((4-((4-(1H-pyrazol-1-yl)-5-(trifluoromethyl)pyrimidin-2-yl)amino)phenyl)sulfonyl)glycine

To a mixture of ((4-((4-(1H-pyrazol-1-yl)-5-(trifluoromethyl)pyrimidin-2-yl)amino)phenyl)sulfonyl)glycinate (144.0 mg, 0.2 mmol) in DCM (2 mL), TFA (0.5 mL, 6.5 mmol) was added and stirred at rt for 3 h. The reaction mixture was concentrated. The residue was diluted with water, extracted with DCM and IPA (v/v=3:1) and concentrated in vacuo. The residue was purified by C18 chromatography (eluting with 52% acetonitrile in water) to afford ((4-((4-(1H-pyrazol-1-yl)-5-(trifluoromethyl)pyrimidin-2-yl)amino)phenyl)sulfonyl)glycine as a white solid.

MS (ES-API positive): 443.0 (M+H)⁺.

¹H NMR (400 MHz, Methanol-d₄) δ 8.90 (s, 1H), 8.59 (d, J=2.7 Hz, 1H), 7.95 (d, J=8.8 Hz, 2H), 7.85 (dt, J=6.1, 2.2 Hz, 3H), 6.62 (dd, J=2.8, 1.6 Hz, 1H), 3.66 (s, 2H).

### EXAMPLES

Examples

| Example | | |
|---|---|---|
| No. | Structure | Mass and ¹H NMR |
| 1 | 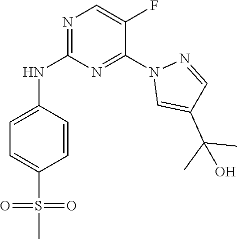 | MS (ES-API positive): 399.3 (M + H)⁺. ¹H NMR (400 MHz, CD₃OD) δ 8.38 (s, 1H), 8.35 (d, J = 4.3 Hz, 1H), 7.86 (s, 1H), 3.99-3.93 (M, 1H), 3.71 (d, J = 12.3 Hz, 2H), 2.97 (td, J = 12.0, 2.1 Hz, 2H), 2.86 (s, 3H), 2.12 (dd, J=12.8, 3.0 Hz, 2H), 1.65 (ddd, J = 12.7, 11.2, 4.0 Hz, 2H), 1.57 (s, 6H). |
| 2 | 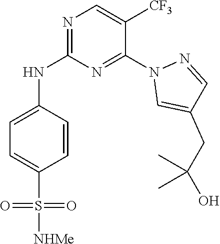 [SMILES: CNS(=O)(=O)c1ccc(Nc2ncc(C(F)(F)F)c(-n3cc(CC(C)(C)O)cn3)n2)cc1] | MS (ES-API positive): 471.0 (M + H)⁺. ¹H NMR (400 MHz, CD₃OD) δ 8.87 (s, 1H), 8.42 (s, 1H), 7.97 (d, J = 8.8 Hz, 2H), 7.82 (d, J = 8.8 Hz, 2H), 7.74 (s, 1H), 2.71 (s, 2H), 2.54 (s, 3H), 1.24 (s, 6H). |
| 3 | 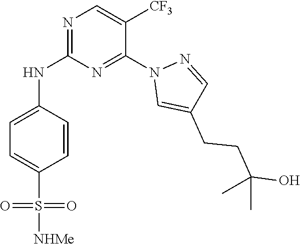 [SMILES: CNS(=O)(=O)c1ccc(Nc2ncc(C(F)(F)F)c(-n3cc(CCC(C)(C)O)cn3)n2)cc1] | MS (ES-API positive): 485.1 (M + H)⁺. ¹H NMR (400 MHz, CD₃OD) δ 8.86 (s, 1H), 8.36 (s, 1H), 7.96 (d, J = 8.8 Hz, 2H), 7.82 (d, J = 8.8 Hz, 2H), 7.73 (s, 1H), 2.70-2.62 (m, 2H), 2.54 (s, 3H), 1.85-1.78 (m, 2H), 1.27 (s, 6H). |
| 4 | 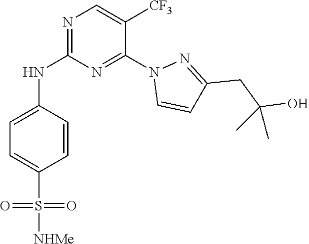 [SMILES: CNS(=O)(=O)c1ccc(Nc2ncc(C(F)(F)F)c(-n3ccc(CC(C)(C)O)n3)n2)cc1] | MS (ES-API positive): 471.3 (M + H)⁺. ¹H NMR (500 MHz, CD₃OD) δ 8.87 (s, 1H), 8.51 (d, J = 2.7 Hz, 1H), 7.96 (d, J = 8.9 Hz, 2H), 7.85-7.79 (m, 2H), 6.51 (d, J = 2.6 Hz, 1H), 2.87 (s, 2H), 2.54 (s, 3H), 1.26 (s, 6H). |
| 5 | 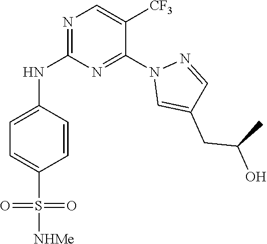 [SMILES: CNS(=O)(=O)c1ccc(Nc2ncc(C(F)(F)F)c(-n3cc(C[C@@H](C)O)cn3)n2)cc1] | MS (ES-API positive): 457.0 (M + H)⁺. ¹H NMR (400 MHz, CD₃OD) δ 8.86 (s, 1H), 8.42 (s, 1H), 7.96 (d, J = 8.7 Hz, 2H), 7.82 (d, J = 8.8 Hz, 2H), 7.75 (s, 1H), 3.97 (t, J = 6.2 Hz, 1H), 2.76-2.64 (m, 2H), 2.54 (s, 3H), 1.22 (d, J = 6.2 Hz, 3H). |
| | Isomer-1 | |
| 6 | 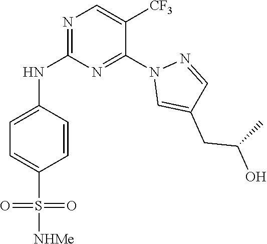 [SMILES: CNS(=O)(=O)c1ccc(Nc2ncc(C(F)(F)F)c(-n3cc(C[C@H](C)O)cn3)n2)cc1] | MS (ES-API positive): 457.0 (M + H)⁺. ¹H NMR (400 MHz, CD₃OD) δ 8.86 (s, 1H), 8.42 (s, 1H), 7.96 (d, J = 8.7 Hz, 2H), 7.82 (d, J = 8.9 Hz, 2H), 7.75 (s, 1H), 3.97 (t, J = 6.1 Hz, 1H), 2.75-2.64 (m, 2H), 2.54 (s, 3H), 1.22 (d, J = 6.2 Hz, 3H). |
| | Isomer-2 | |
| 7 | 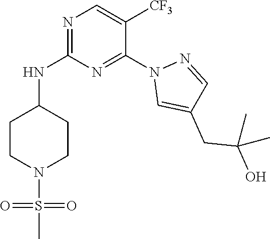 [SMILES: CC(C)(O)Cc1cnn(-c2nc(NC3CCN(S(C)(=O)=O)CC3)ncc2C(F)(F)F)c1] | MS (ES-API positive): 463.1 (M + H)⁺. ¹HNMR (400 MHz, CD₃OD) δ 8.72-8.59 (m, 1H), 8.42- 8.26 (m, 1H), 7.81-7.62 (m, 1H), 4.15-3.97 (m, 1H), 3.79-3.69 (m, 2H), 3.04-2.95 (m, 2H), 2.90-2.82 (m, 3H), 2.76-2.63 (m, 2H), 2.21-2.10 (m, 2H), 1.78-1.58 (m, 2H), 1.28-1.16 (m, 6H). |
| 8 | 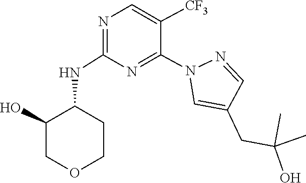 [SMILES: CC(C)(O)Cc1cnn(-c2nc(N[C@@H]3CCOC[C@H]3O)ncc2C(F)(F)F)c1] | MS (ES-API positive): 402.1 (M + H)⁺. ¹H NMR (500 MHz, CD₃OD) δ 8.66-8.56 (m, 1H), 8.36- 8.29 (m, 1H), 7.72-7.64 (m, 1H), 4.13-4.04 (m, 1H), 3.97 (dd, J = 4.8, 11.2 Hz, 1H), 3.94-3.88 (m, 1H), 3.68- 3.57 (m, 1H), 3.55-3.43 (m, 1H), 3.27-3.17 (m, 1H), 2.74-2.64 (m, 2H), 2.12-2.00 (m, 2H), 1.71-1.57 (m, 2H), 1.36-1.28 (m, 3H), 1.21 (s, 6H). |
| | Isomer-1 | |
| 9 | 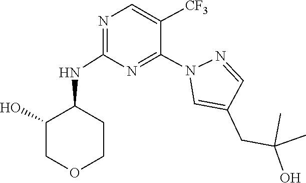 [SMILES: CC(C)(O)Cc1cnn(-c2nc(N[C@H]3CCOC[C@@H]3O)ncc2C(F)(F)F)c1] | MS (ES-API positive): 402.1 (M + H)⁺. ¹H NMR (500 MHz, CD₃OD) δ 8.65-8.54 (m, 1H), 8.37- 8.25 (m, 1H), 7.76-7.62 (m, 1H), 4.11-4.00 (m, 1H), 3.98-3.93 (m, 1H), 3.93-3.87 (m, 1H), 3.63-3.54 (m, 1H), 3.53-3.44 (m, 1H), 3.25-3.16 (m, 1H), 2.70-2.63 (m, 2H), 2.11-1.99 (m, 1H), 1.70-1.55 (m, 1H), 1.35- 1.25 (m, 2H), 1.21-1.18 (m, 6H). |
| | Isomer-2 | |
| 10 | 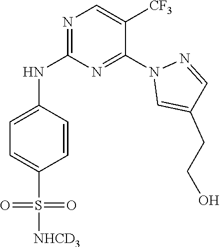 | MS (ES-API positive): 446.2 (M + H)⁺. ¹H NMR (400 MHz, DMSO-d₆) δ 10.82 (s, 1H), 8.96 (s, 1H), 8.44 (s, 1H), 7.96 (d, J = 8.5 Hz, 2H), 7.86 (s, 1H), 7.78 (d, J = 8.5 Hz, 2H), 7.32 (s, 1H), 4.79 (t, J = 5.1 Hz, 1H), 3.63 (q, J = 5.8, 5.1 Hz, 2H), 2.68 (t, J = 6.6 Hz, 2H). MS (ES-API positive): 467.1 (M + H)⁺. |
| 11 | 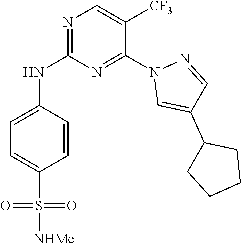 [SMILES: CNS(=O)(=O)c1ccc(Nc2ncc(C(F)(F)F)c(-n3cc(C4CCCC4)cn3)n2)cc1] | MS (ES-API positive): 467.1 (M + H)⁺. ¹H NMR (400 MHz, CD₃OD) δ 8.86 (s, 1H), 8.34 (s, 1H), 7.96 (d, J = 8.8 Hz, 2H), 7.81 (d, J = 8.8 Hz, 2H), 7.74 (s, 1H), 3.04 (t, J = 8.2 Hz, 1H), 2.54 (s, 3H), 2.18-2.02 (m, 2H), 1.92-1.66 (m, 4H), 1.66-1.53 (m, 2H). MS (ES-API positive): 465.2 (M + H)⁺. |
| 12 | 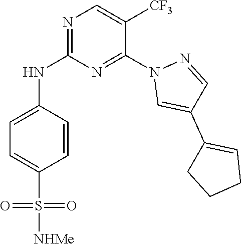 [SMILES: CNS(=O)(=O)c1ccc(Nc2ncc(C(F)(F)F)c(-n3cc(C4=CCCC4)cn3)n2)cc1] | MS (ES-API positive): 465.2 (M + H)⁺. ¹H NMR (500 MHz, CD₃OD) δ 8.87 (s, 1H), 8.44 (s, 1H), 8.00 (s, 1H), 7.97 (d, J = 8.9 Hz, 2H), 7.84-7.80 (m, 2H), 6.14 (t, J = 2.1 Hz, 1H), 2.69-2.65 (m, 2H), 2.55 (d, J = 2.6 Hz, 1H), 2.54 (s, 3H), 2.53-2.49 (m, 1H), 2.08-2.01 (m, 2H). |
| 13 | 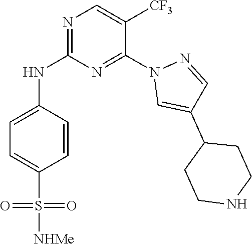 [SMILES: CNS(=O)(=O)c1ccc(Nc2ncc(C(F)(F)F)c(-n3cc(C4CCNCC4)cn3)n2)cc1] | MS (ES-API positive): 482.2 (M + H)⁺. ¹H NMR (400 MHz, CD₃OD) δ 8.89 (s, 1H), 8.43 (s, 1H), 7.94 (s, 2H), 7.84 (s, 3H), 3.58-3.55 (m, 1H), 3.48 (br s, 1H), 3.17 (d, J = 2.5 Hz, 2H), 3.09-2.97 (m, 1H), 2.54 (s, 3H), 2.27 (d, J = 13.0 Hz, 2H), 1.86 (d, J = 11.2 Hz, 2H). |
| 14 | 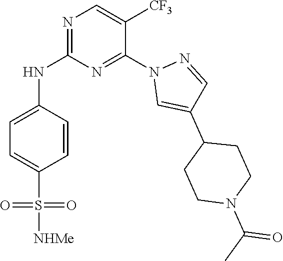 [SMILES: CNS(=O)(=O)c1ccc(Nc2ncc(C(F)(F)F)c(-n3cc(C4CCN(C(C)=O)CC4)cn3)n2)cc1] | MS (ES-API positive): 524.1 (M + H)⁺. ¹H NMR (400 MHz, CD₃OD) δ 8.86 (s, 1H), 8.38 (s, 1H), 7.95 (d, J = 8.8 Hz, 2H), 7.86-7.77 (m, 3H), 4.72-4.52 (m, 1H), 4.15-3.86 (m, 1H), 3.27-3.21 (m, 1H), 2.93 (s, 1H), 2.84-2.71 (m, 1H), 2.55 (s, 3H), 2.14 (s, 3H), 2.11- 1.97 (m, 2H), 1.71-1.47 (m, 2H). |
| 15 | 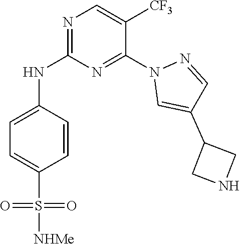 [SMILES: CNS(=O)(=O)c1ccc(Nc2ncc(C(F)(F)F)c(-n3cc(C4CNC4)cn3)n2)cc1] | MS (ES-API positive): 454.1 (M + H)⁺. ¹H NMR (500 MHz, CD₃OD) δ 8.91 (s, 1H), 8.59 (s, 1H), 8.00 (s, 1H), 7.96 (d, J = 8.9 Hz, 2H), 7.87-7.82 (m, 2H), 4.46-4.40 (m, 2H), 4.31 (q, J = 8.2 Hz, 1H), 4.26-4.20 (m, 2H), 2.55 (s, 3H). |
| 16 | 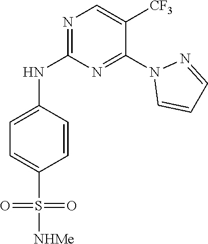 [SMILES: CNS(=O)(=O)c1ccc(Nc2ncc(C(F)(F)F)c(-n3cccn3)n2)cc1] | MS (ES-API positive): 399.0 (M + H)⁺. ¹H NMR (400 MHz, DMSO-d₆) δ 10.85 (s, 1H), 8.99 (s, 1H), 8.61 (s, 1H), 7.96 (d, J = 11.2 Hz, 3H), 7.78 (d, J = 8.5 Hz, 2H), 7.33 (s, 1H), 6.69 (s, 1H), 2.42 (s, 3H). |
| 17 | 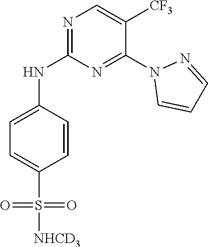 | MS (ES-API positive): 402.2 (M + H)⁺. ¹H NMR (400 MHz, DMSO-d₆) δ 10.86 (s, 1H), 9.00 (s, 1H), 8.62 (d, J = 2.8 Hz, 1H), 8.04-7.88 (m, 3H), 7.88- 7.58 (m, 2H), 7.31 (s, 1H), 6.71-6.69 (m, 1H). |
| 18 | 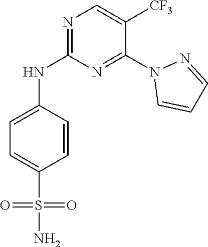 [SMILES: NS(=O)(=O)c1ccc(Nc2ncc(C(F)(F)F)c(-n3cccn3)n2)cc1] | MS (ES-API positive): 385.0 (M + H)⁺. ¹H NMR (400 MHz, DMSO-d₆) δ 10.77 (s, 1H), 8.94 (s, 1H), 8.56 (d, J = 2.7 Hz, 1H), 7.93 (s, 1H), 7.87 (d, J = 8.5 Hz, 2H), 7.79 (d, J = 8.6 Hz, 2H), 7.25 (s, 2H), 6.65 (s, 1H). |
| 19 | 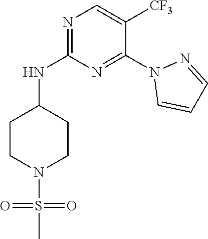 [SMILES: CS(=O)(=O)N1CCC(Nc2ncc(C(F)(F)F)c(-n3cccn3)n2)CC1] | MS (ES-API positive): 391.2 (M + H)⁺. ¹H NMR (400 MHz, DMSO-d₆) δ 8.90 -8.10 (m, 3H), 7.91 (d, J = 6.1 Hz, 1H), 6.63 (d, J = 6.1 Hz, 1H), 4.13-3.89 (m, 1H), 3.55 (d, J = 11.6 Hz, 2H), 3.14-2.68 (m, 5H), 1.99 (d, J = 12.5 Hz, 2H), 1.59 (q, J = 11.9 Hz, 2H). |
| 20 | 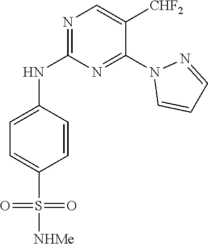 [SMILES: CNS(=O)(=O)c1ccc(Nc2ncc(C(F)F)c(-n3cccn3)n2)cc1] | MS (ES-API positive): 381.0 (M + H)⁺. ¹H NMR (400 MHz, Methanol-d₄) δ 10.67 (s, 1H), 8.92 (s, 1H), 8.72 (d, J = 2.8 Hz, 1H), 8.05-8.01 (m, 1H), 8.00- 7.93 (m, 2H), 7.81-7.76 (m, 2H), 7.36-7.26 (m, 1H), 6.75-6.70 (m, 1H), 2.46 (d, J = 4.8 Hz, 3H). |
| 21 | 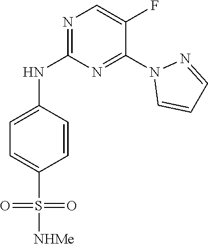 [SMILES: CNS(=O)(=O)c1ccc(Nc2ncc(F)c(-n3cccn3)n2)cc1] | MS (ES-API positive): 349.0 (M + H)⁺. ¹H NMR (400 MHz, DMSO-d₆) δ 10.38 (s, 1H), 9.09- 8.71 (m, 1H), 8.60 (s, 1H), 8.21-7.85 (m, 3H), 7.72 (d, J = 8.4 Hz, 2H), 7.27 (d, 1H), 6.71 (s, 1H), 2.39 (d, J = 4.9 Hz, 3H). |
| 22 | 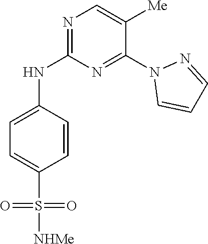 [SMILES: CNS(=O)(=O)c1ccc(Nc2ncc(C)c(-n3cccn3)n2)cc1] | MS (ES-API positive): 345.2 (M + H)⁺. ¹H NMR (400 MHz, DMSO-d₆) δ 10.18 (s, 1H), 8.64 (dd, J = 2.7, 0.7 Hz, 1H), 8.56 (d, J = 0.8 Hz, 1H), 8.20- 7.88 (m, 3H), 7.72 (d, J = 8.9 Hz, 2H), 7.24 (q, J = 5.1 Hz, 1H), 6.66-6.65 (m, 1H), 2.50 (s, 3H), 2.40 (d, J = 5.1 Hz, 3H). |
| 23 | 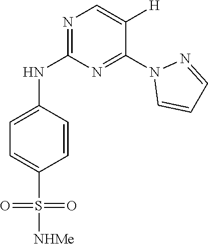 [SMILES: CNS(=O)(=O)c1ccc(Nc2nccc(-n3cccn3)n2)cc1] | MS (ES-API positive): 330.9 (M + H)⁺. ¹H NMR (400 MHz, DMSO-d₆) δ 10.32 (s, 1H), 8.78- 8.50 (m, 2H), 8.03-7.97 (m, 2H), 7.95 (d, J = 4.0 Hz, 1H), 7.75 (d, J = 8.0 Hz, 2H), 7.38 (d, J = 4.0 Hz, 1H), 7.27 (q, J = 16 Hz, 1H), 6.69 (dd, J = 1.0, 4 Hz, 1H), 2.41 (d, J = 8.0 Hz, 3H). |
| 24 | 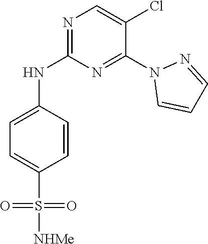 [SMILES: CNS(=O)(=O)c1ccc(Nc2ncc(Cl)c(-n3cccn3)n2)cc1] | MS (ES-API positive): 365.0 (M + H)⁺. ¹H NMR (400 MHz, DMSO-d₆) δ 10.53 (s, 1H), 8.81- 8.76 (m, 1H), 8.61-8.54 (m, 1H), 8.00-7.89 (m, 3H), 7.78-7.70 (m, 2H), 7.32-7.25 (m, 1H), 6.72-6.67 (m, 1H), 2.43-2.39 (m, 3H). |
| 25 | 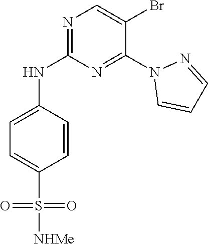 [SMILES: CNS(=O)(=O)c1ccc(Nc2ncc(Br)c(-n3cccn3)n2)cc1] | MS (ES-API positive): 409.0 & 411.0 (M + H)⁺. ¹H NMR (500 MHz, DMSO-d₆) δ 10.51 (s, 1H), 8.86 (s, 1H), 8.54 (d, J = 2.6 Hz, 1H), 7.99-7.86 (m, 3H), 7.73 (d, J = 8.7 Hz, 2H), 7.27 (q, J = 4.9 Hz, 1H), 6.67 (dd, J = 1.7, 2.4 Hz, 1H), 2.40 (d, J = 5.0 Hz, 3H). |
| 26 | 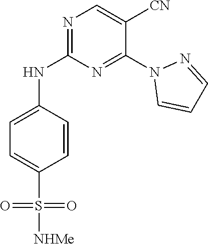 [SMILES: CNS(=O)(=O)c1ccc(Nc2ncc(C#N)c(-n3cccn3)n2)cc1] | MS (ES-API positive): 355.9 (M + H)⁺. ¹H NMR (400 MHz, DMSO-d₆) δ 11.12-10.89 (m, 1H), 9.02 (s, 1H), 8.66 (d, J = 2.4 Hz, 1H), 8.08 (d, J = 1.0 Hz, 1H), 7.98-7.94 (m, 2H), 7.84-7.78 (m, 2H), 7.38-7.33 (m, 1H), 6.78-6.76 (m, 1H), 2.43 (d, J = 5.1 Hz, 3H). |
| 27 | 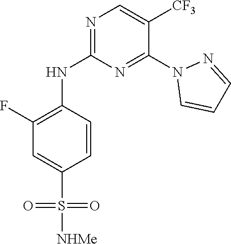 [SMILES: CNS(=O)(=O)c1ccc(Nc2ncc(C(F)(F)F)c(-n3cccn3)n2)c(F)c1] | MS (ES-API positive): 417.0 (M + H)⁺. ¹H NMR (400 MHz, DMSO-d₆) δ 9.07 (s, 1H), 8.54 (d, J = 2.8 Hz, 1H), 7.99 (s, 1H), 7.64 (d, J = 11.2 Hz, 1H), 7.58 (d, J = 8.4 Hz, 1H), 6.78 (t, J = 8.4 Hz, 1H), 6.71 (s, 1H), 6.26 (s, 2H), 3.66 (s, 3H). |
| 28 | 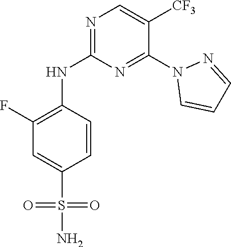 [SMILES: NS(=O)(=O)c1ccc(Nc2ncc(C(F)(F)F)c(-n3cccn3)n2)c(F)c1] | MS (ES-API positive): 403.0 (M + H)⁺. ¹H NMR (400 MHz, DMSO-d₆) δ 10.45 (s, 1H), 8.94 (s, 1H), 8.45 (d, J = 2.8 Hz, 1H), 8.01-7.95 (m, 2H), 7.70 (dt, J = 10.3, 2.2 Hz, 2H), 7.47 (s, 2H), 6.66 (dd, J = 2.8, 1.6 Hz, 1H). |
| 29 | 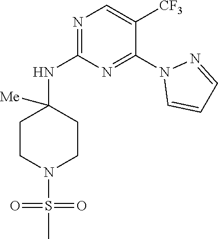 [SMILES: CC1(Nc2ncc(C(F)(F)F)c(-n3cccn3)n2)CCN(S(C)(=O)=O)CC1] | MS (ES-API positive): 405.2 (M + H)⁺. ¹H NMR (400 MHz, DMSO-d₆) δ 8.75 (d, J = 16.4 Hz, 1H), 8.46 (s, 1H), 8.05-7.85 (m, 2H), 6.69-6.60 (m, 1H), 3.06-2.96 (m, 2H), 2.85 (s, 3H), 2.47-2.39 (m, 2H), 1.74-1.62 (m, 2H), 1.45 (s, 3H), 1.26-1.22 (m, 2H). |
| 30 | 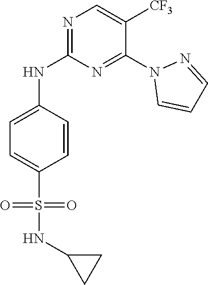 [SMILES: O=S(=O)(NC1CC1)c1ccc(Nc2ncc(C(F)(F)F)c(-n3cccn3)n2)cc1] | MS (ES-API positive): 425.0 (M + H)⁺. ¹H NMR (400 MHz, DMSO-d₆) δ 10.85 (s, 1H), 9.00 (s, 1H), 8.61 (s, 1H), 8.16-7.91 (m, 3H), 7.91-7.71 (m, 3H), 6.69 (s, 1H), 2.22-2.00 (m, 1H), 0.58-0.41 (m, 2H), 0.42-0.29 (m, 2H). |
| 31 | 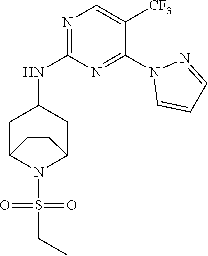 [SMILES: CCS(=O)(=O)N1C2CCC1CC(Nc1ncc(C(F)(F)F)c(-n3cccn3)n1)C2] | MS (ES-API positive): 431.2 (M + H)⁺. ¹H NMR (400 MHz, CDCl₃) δ 8.65 (d, J = 18.7 Hz, 1H), 8.35 (d, J = 19.5 Hz, 1H), 7.85 (s, 1H), 7.30 (d, J = 2.2 Hz, 1H), 6.51 (s, 1H), 4.30 (s, 2H), 3.14-2.98 (m, 2H), 2.39 (s, 1H), 2.29-1.85 (m, 6H), 1.72 (s, 2H), 1.40- 1.34 (m, 3H). |
| 32 | 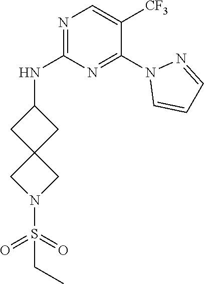 [SMILES: CCS(=O)(=O)N1CC2(CC(Nc3ncc(C(F)(F)F)c(-n4cccn4)n3)C2)C1] | MS (ES-API positive): 417.2 (M + H)⁺. ¹H NMR (400 MHz, DMSO-d₆) δ 8.76-8.36 (m, 3H), 7.90 (d, J = 5.6 Hz, 1H), 6.65-6.57 (m, 1H), 4.37-4.24 (m, 1H), 3.95 (d, J = 10.2 Hz, 2H), 3.82 (s, 2H), 3.07 (t, J = 7.3 Hz, 2H), 2.60 (s, 2H), 2.23 (dd, J = 14.7, 6.4 Hz, 2H), 1.20 (t, J = 7.4 Hz, 3H). |
| 33 | 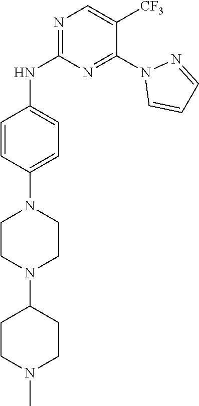 [SMILES: CN1CCC(N2CCN(c3ccc(Nc4ncc(C(F)(F)F)c(-n5cccn5)n4)cc3)CC2)CC1] | MS (ES-API positive): 487.2 (M + H)⁺. ¹H NMR (400 MHz, DMSO-d₆) δ 9.14 (s, 1H), 8.65 (s, 1H), 8.34 (d, J = 2.8 Hz, 1H), 8.20 (s, 1H), 7.83 (s, 1H), 7.44 (s, 1H), 7.42 (s, 1H), 6.98 (s, 1H), 6.95 (s, 1H), 6.58-6.53 (m, 1H), 3.20-3.08 (m, 4H), 2.93-2.84 (m, 2H), 2.67-2.59 (m, 4H), 2.22 (s, 3H), 2.05-1.95 (m, 2H), 1.85-1.73 (m, 2H), 1.56-1.39 (m, 2H). |
| 34 | 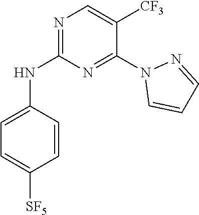 | MS (ES-API positive): 432.0 (M + H)⁺. ¹H NMR (400 MHz, DMSO-d₆) δ 10.90 (s, 1H), 9.01 (d, J = 2.0 Hz, 1H), 8.63 (t, J = 2.4 Hz, 1H), 8.04-7.85 (m, 5H), 6.70 (s, 1H). |
| 35 | 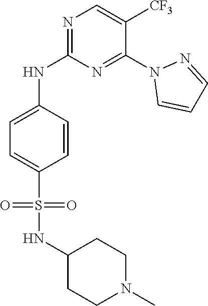 [SMILES: CN1CCC(NS(=O)(=O)c2ccc(Nc3ncc(C(F)(F)F)c(-n4cccn4)n3)cc2)CC1] | MS (ES-API positive): 482.0 (M + H)⁺. ¹H NMR (400 MHz, Methanol-d₄) δ 8.92 (s, 1H), 8.60 (s, 2H), 8.01 (d, J = 6.4 Hz, 2H), 7.09 (d, J = 7.2 Hz, 2H), 6.64 (s, 1H), 3.51-3.35 (m, 3H), 3.29-2.97 (m, 2H), 2.88 (s, 1H), 2.81 (s, 2H), 2.08-1.99 (m, 2H), 1.78-1.64 (m, 2H). |
| 36 | 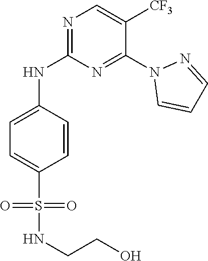 [SMILES: O=S(=O)(NCCO)c1ccc(Nc2ncc(C(F)(F)F)c(-n3cccn3)n2)cc1] | MS (ES-API positive): 429.1 (M + H)⁺. ¹H NMR (400 MHz, DMSO-d₆) δ 10.86 (s, 1H), 9.01 (s, 1H), 8.62 (d, J = 2.8 Hz, 1H), 8.13-7.88 (m, 3H), 7.80 (d, J = 8.5 Hz, 2H), 7.50 (t, J = 6.0 Hz, 1H), 6.71 (s, 1H), 4.70 (t, J = 5.6 Hz, 1H), 3.40-3.38 (m, 2H), 2.80 (q, J = 6.2 Hz, 2H). |
| 37 | 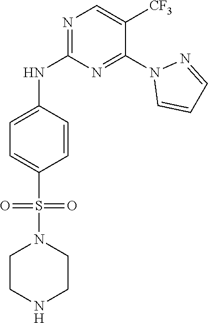 [SMILES: O=S(=O)(c1ccc(Nc2ncc(C(F)(F)F)c(-n3cccn3)n2)cc1)N1CCNCC1] | MS (ES-API positive): 454.0 (M + H)⁺. ¹H NMR (400 MHz, DMSO-d₆) δ 11.00 (s, 1H), 9.03 (s, 1H), 8.64 (d, J = 4.0 Hz, 1H), 8.57 (s, 1H), 8.06 (d, J = 8.0 Hz, 2H), 8.00 (d, J = 1.0 Hz, 1H), 7.81 (d, J = 8.0 Hz, 2H), 6.72 (dd, J = 1.6, 2.4 Hz, 1H), 3.20 (d, J = 4.6 Hz, 4H), 3.09 (s, 4H). |
| 38 | 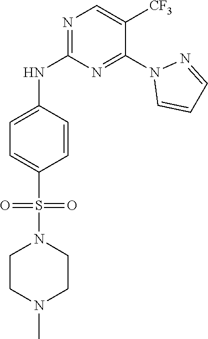 [SMILES: CN1CCN(S(=O)(=O)c2ccc(Nc3ncc(C(F)(F)F)c(-n4cccn4)n3)cc2)CC1] | MS (ES-API positive):468.0 (M + H)⁺. ¹H NMR (400 MHz, DMSO-d₆) δ 11.00 (s, 1H), 9.04 (s, 1H), 8.64 (d, J = 4.0 Hz, 1H), 8.13-7.95 (m, 3H), 7.81 (d, J = 8.0 Hz, 2H), 6.72 (dd, J = 4.0, 1.0 Hz, 1H), 3.56 (s, 4H), 3.32-2.93 (m, 4H), 2.77 (s, 3H). |
| 39 | 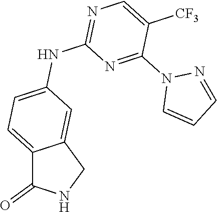 [SMILES: O=C1NCc2cc(Nc3ncc(C(F)(F)F)c(-n4cccn4)n3)ccc21] | MS (ES-API positive): 361.1 (M + H)⁺. ¹H NMR (400 MHz, CDCl₃) δ 8.82-8.75 (m, 1H), 8.43- 8.34 (m, 1H), 7.99-7.90 (m, 1H), 7.88-7.77 (m, 2H), 7.70-7.56 (m, 1H), 7.52-7.47 (m, 1H), 6.55-6.42 (m, 1H), 6.37-6.24 (m, 1H), 4.37-4.37 (m, 1H), 4.48-4.34 (m, 1H). |
| 40 | 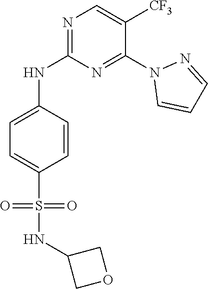 [SMILES: O=S(=O)(NC1COC1)c1ccc(Nc2ncc(C(F)(F)F)c(-n3cccn3)n2)cc1] | MS (ES-API positive): 440.8 (M + H)⁺. ¹H NMR (400 MHz, Methanol-d₄) δ 8.90 (s, 1H), 8.57 (dd, J =2.8, 0.7 Hz, 1H), 7.99-7.92 (m, 2H), 7.85 (dd, J = 1.6, 0.7 Hz, 1H), 7.83-7.79 (m, 2H), 6.61 (dd, J = 2.8, 1.6 Hz, 1H), 4.69-4.61 (m, 2H), 4.55-4.44 (m, 1H), 4.39-4.36 (m, 2H). |
| 41 | 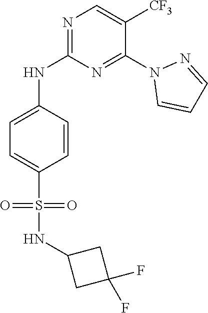 [SMILES: O=S(=O)(NC1CC(F)(F)C1)c1ccc(Nc2ncc(C(F)(F)F)c(-n3cccn3)n2)cc1] | MS (ES-API positive): 475.0 (M + H)⁺. ¹H NMR (400 MHz, Methanol-d₄) δ 8.91 (s, 1H), 8.58 (d, J = 2.6 Hz, 1H), 7.97 (d, J = 8.8 Hz, 2H), 7.90-7.79 (m, 3H), 6.61 (dd, J = 1.6, 2.7 Hz, 1H), 3.74-3.58 (m, 1H), 2.81-2.65 (m, 2H), 2.50-2.31 (m, 2H). |
| 42 | 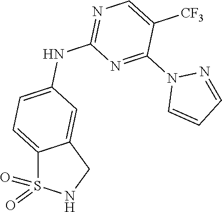 [SMILES: O=S1(=O)NCc2cc(Nc3ncc(C(F)(F)F)c(-n4cccn4)n3)ccc21] | MS (ES-API positive): 396.9 (M + H)⁺. ¹H NMR (400 MHz, DMSO-d₆) δ 10.90-10.74 (m, 1H), 9.02 (s, 1H), 8.60-8.45 (m, 1H), 8.34-8.16 (m, 1H), 7.98 (s, 1H), 7.94-7.82 (m, 2H), 7.60-7.50 (m, 1H), 6.70 (s, 1H), 4.38 (d, J = 5.0 Hz, 2H). |
| 43 | 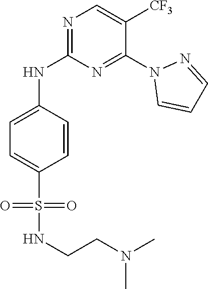 [SMILES: CN(C)CCNS(=O)(=O)c1ccc(Nc2ncc(C(F)(F)F)c(-n3cccn3)n2)cc1] | MS (ES-API positive): 456.0 (M + H)⁺. ¹H NMR (400 MHz, Methanol-d₄) δ 8.93 (s, 1H), 8.60 (d, J = 2.7 Hz, 1H), 8.05-8.01 (m, 2H), 7.93-7.86 (m, 3H), 6.66-6.61 (m, 1H), 3.31-3.28 (m, 2H), 3.25-3.19 (m, 2H), 2.96 (s, 6H). |
| 44 | 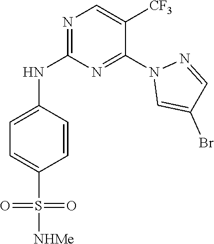 [SMILES: CNS(=O)(=O)c1ccc(Nc2ncc(C(F)(F)F)c(-n3cc(Br)cn3)n2)cc1] | MS (ES-API positive): 477.0 & 479.0 (M + H)⁺. ¹H NMR (400 MHz, Methanol-d₄) δ 8.94 (s, 1H), 8.67 (s, 1H), 7.96 (s, 2H), 7.87 (d, J = 5.8 Hz, 3H), 2.56 (s, 3H). |
| 45 | 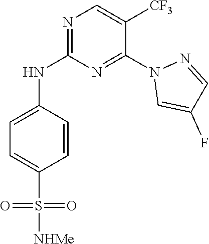 [SMILES: CNS(=O)(=O)c1ccc(Nc2ncc(C(F)(F)F)c(-n3cc(F)cn3)n2)cc1] | MS (ES-API positive): 417.2 (M + H)⁺. ¹H NMR (400 MHz, DMSO-d₆) δ 11.01 (s, 1H), 9.14 (s, 1H), 9.11 (s, 1H), 8.45 (s, 1H), 7.97 (s, 1H), 7.95 (s, 1H), 7.79 (s, 1H), 7.77 (s, 1H), 7.37 (q, J = 5.2 Hz, 1H), 2.41 (d, J = 5.2 Hz, 3H). |
| 46 | 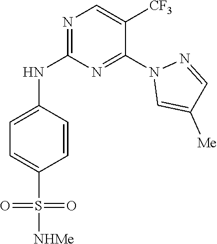 [SMILES: CNS(=O)(=O)c1ccc(Nc2ncc(C(F)(F)F)c(-n3cc(C)cn3)n2)cc1] | MS (ES-API positive): 413.0 (M + H)⁺. ¹H NMR (500 MHz, Methanol-d₄) δ 8.87 (s, 1H), 8.35 (s, 1H), 8.00-7.95 (m, 2H), 7.88-7.82 (m, 2H), 7.70 (s, 1H), 2.56 (s, 3H), 2.21 (d, J = 0.6 Hz, 3H). |
| 47 | 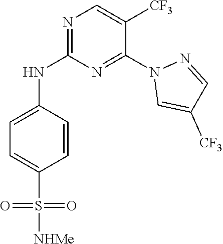 [SMILES: CNS(=O)(=O)c1ccc(Nc2ncc(C(F)(F)F)c(-n3cc(C(F)(F)F)cn3)n2)cc1] | MS (ES-API positive): 467.0 (M + H)⁺. ¹H NMR (400 MHz, DMSO-d₆) δ 11.04 (s, 1H), 9.11 (s, 1H), 8.76 (s, 1H), 7.97 (s, 1H), 7.95 (s, 1H), 7.80 (s, 1H), 7.77 (s, 1H), 7.36 (q, J = 4.8 Hz, 1H), 7.18 (d, J = 2.8 Hz, 1H), 2.41 (d, J = 4.8 Hz, 3H). |
| 48 | 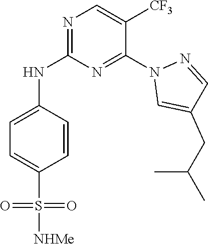 [SMILES: CNS(=O)(=O)c1ccc(Nc2ncc(C(F)(F)F)c(-n3cc(CC(C)C)cn3)n2)cc1] | MS (ES-API positive): 455.0 (M + H)⁺. ¹H NMR (400 MHz, Methanol-d₄) δ 8.86 (s, 1H), 8.32 (s, 1H), 7.96 (d, J = 8.8 Hz, 2H), 7.81 (d, J = 8.8 Hz, 2H), 7.68 (s, 1H), 2.53 (s, 3H), 2.46 (d, J = 6.9 Hz, 2H), 1.87 (m, 1H), 0.97 (d, J = 6.7 Hz, 6H). |
| 49 | 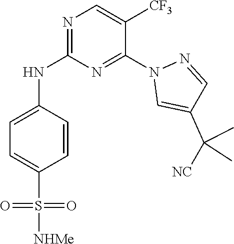 [SMILES: CNS(=O)(=O)c1ccc(Nc2ncc(C(F)(F)F)c(-n3cc(C(C)(C)C#N)cn3)n2)cc1] | MS (ES-API positive): 466.1 (M + H)⁺. ¹H NMR (400 MHz, Methanol-d₄) δ 8.92 (s, 1H), 8.63 (s, 1H), 8.04-7.92 (m, 3H), 7.82 (d, J = 8.8 Hz, 2H), 2.54 (s, 3H), 1.77 (s, 6H) |
| 50 | 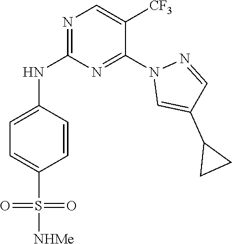 [SMILES: CNS(=O)(=O)c1ccc(Nc2ncc(C(F)(F)F)c(-n3cc(C4CC4)cn3)n2)cc1] | MS (ES-API positive): 439.1 (M + H)⁺. ¹H NMR (400 MHz, Methanol-d₄) δ 8.87 (s, 1H), 8.32 (s, 1H), 7.98 (d, J = 8.7 Hz, 2H), 7.84 (d, J = 8.7 Hz, 2H), 7.67 (s, 1H), 2.56 (s, 3H), 1.90-1.81 (m, 1H), 1.03-0.94 (m, 2H), 0.71-0.62 (m, 2H). |
| 51 | 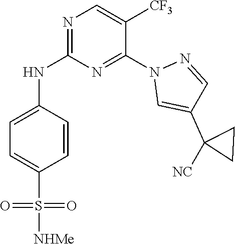 [SMILES: CNS(=O)(=O)c1ccc(Nc2ncc(C(F)(F)F)c(-n3cc(C4(C#N)CC4)cn3)n2)cc1] | MS (ES-API positive): 464.1 (M + H)⁺. ¹H NMR (400 MHz, Methanol-d₄) δ 8.93 (s, 1H), 8.65- 8.60 (m, 1H), 8.02-7.97 (m, 2H), 7.87-7.83 (m, 2H), 7.82-7.79 (m, 1H), 2.62-2.52 (m, 3H), 1.80-1.72 (m, 2H), 1.58-1.43 (m, 2H). |
| 52 | 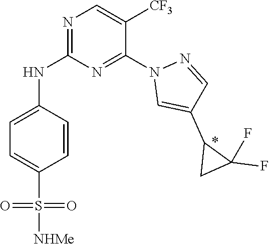 [SMILES: CNS(=O)(=O)c1ccc(Nc2ncc(C(F)(F)F)c(-n3cc(C4CC4(F)F)cn3)n2)cc1] | MS (ES-API positive): 474.4 (M + H)⁺. ¹H NMR (400 MHz, Methanol-d₄) δ 8.91 (s, 1H), 8.50 (s, 1H), 7.98 (d, J = 8.8 Hz, 2H), 7.90-7.75 (m, 3H), 2.83 (dt, J = 7.7, 12.2 Hz, 1H), 2.56 (s, 3H), 2.04-1.91 (m, 1H), 1.52-1.47 (m, 1H). |
| | Isomer-1 | |
| 53 | 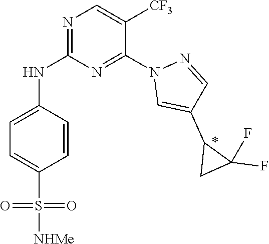 [SMILES: CNS(=O)(=O)c1ccc(Nc2ncc(C(F)(F)F)c(-n3cc(C4CC4(F)F)cn3)n2)cc1] | MS (ES-API positive): 474.4 (M + H)⁺. ¹H NMR (400 MHz, Methanol-d₄) δ 8.91 (s, 1H), 8.50 (s, 1H), 7.98 (d, J = 8.8 Hz, 2H), 7.90-7.75 (m, 3H), 2.83 (dt, J = 7.7, 12.2 Hz, 1H), 2.56 (s, 3H), 2.04-1.91 (m, 1H), 1.52-1.47 (m, 1H). |
| | Isomer-2 | |
| 54 | 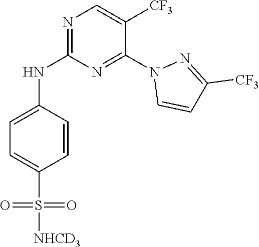 | MS (ES-API positive): 470.0 (M + H)⁺. ¹H NMR (400 MHz, DMSO-d₆) δ 11.02 (s, 1H), 9.10 (s, 1H), 8.75 (s, 1H), 7.96 (s, 1H), 7.94 (s, 1H), 7.79 (s, 1H), 7.77 (s, 1H), 7.32 (s, 1H), 7.18 (d, J = 2.8 Hz, 1H). |
| 55 | 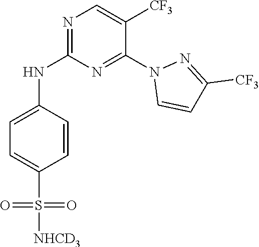 | MS (ES-API positive): 416.2 (M + H)⁺. ¹H NMR (400 MHz, DMSO-d₆) δ 10.34 (s, 1H), 8.79 (d, J = 2.8 Hz, 1H), 8.67 (s, 1H), 7.95 (s, 1H), 7.93 (s, 1H), 7.74 (s, 1H), 7.72 (s, 1H), 7.24 (s, 1H), 7.14 (d, J = 2.8 Hz, 1H), 2.45 (s, 3H). |
| 56 | 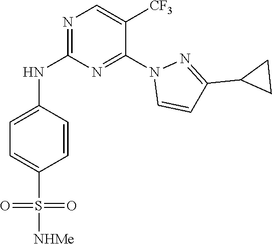 [SMILES: CNS(=O)(=O)c1ccc(Nc2ncc(C(F)(F)F)c(-n3ccc(C4CC4)n3)n2)cc1] | MS (ES-API positive): 439.0 (M + H)⁺. ¹H NMR (400 MHz, Methanol-d₄) δ 10.75 (s, 1H), 8.93 (s, 1H), 8.48 (d, J = 2.7 Hz, 1H), 7.94 (d, J = 8.8 Hz, 2H), 7.77 (d, J = 8.8 Hz, 2H), 7.31 (q, J = 5.1 Hz, 1H), 6.50 (d, J = 2.7 Hz, 1H), 2.43-2.37 (m, 3H), 2.03-1.88 (m, 1H), 1.02-0.91 (m, 2H), 0.85-0.69 (m, 2H). |
| 57 | 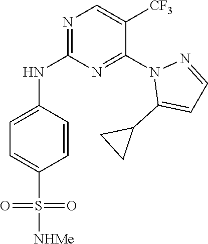 [SMILES: CNS(=O)(=O)c1ccc(Nc2ncc(C(F)(F)F)c(-n3nccc3C3CC3)n2)cc1] | MS (ES-API positive): 439.0 (M + H)⁺. ¹H NMR (400 MHz, Methanol-d₄) δ 9.00 (s, 1H), 8.00 (d, J = 8.8 Hz, 2H), 7.82 (d, J = 8.8 Hz, 2H), 7.63 (d, J = 1.5 Hz, 1H), 6.16 (d, J = 1.3 Hz, 1H), 2.54 (s, 3H), 2.20 (s, 1H), 1.00-0.94 (m, 2H), 0.77-0.71 (m, 2H). |
| 58 | 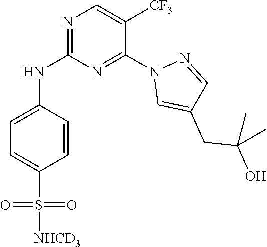 | MS (ES-API positive):474.0 (M + H)⁺. ¹H NMR (400 MHz, Methanol-d₄) δ 8.91-8.86 (m, 1H), 8.47-8.40 (m, 1H), 8.01-7.96 (m, 2H), 7.87-7.81 (m, 2H), 7.77-7.74 (m, 1H), 2.77-2.69 (m, 2H), 1.28-1.23 (m, 6H). |
| 59 | 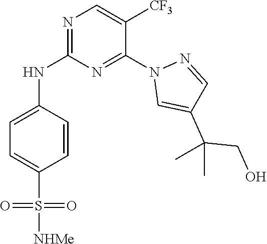 [SMILES: CNS(=O)(=O)c1ccc(Nc2ncc(C(F)(F)F)c(-n3cc(C(C)(C)CO)cn3)n2)cc1] | MS (ES-API positive): 471.1 (M + H)⁺. ¹H NMR (400 MHz, Methanol-d₄) δ 8.85 (s, 1H), 8.42 (s, 1H), 7.96 (d, J=8.8 Hz, 2H), 7.87-7.76 (m, 3H), 3.52 (s, 2H), 2.54 (s, 3H), 1.31 (s, 6H). |
| 60 | 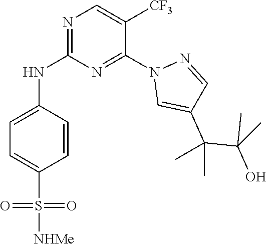 [SMILES: CNS(=O)(=O)c1ccc(Nc2ncc(C(F)(F)F)c(-n3cc(C(C)(C)C(C)(C)O)cn3)n2)cc1] | MS (ES-API positive): 499.1 (M + H)⁺. ¹H NMR (400 MHz, Methanol-d₄) δ 8.86 (s, 1H), 8.40 (s, 1H), 8.02-7.93 (m, 2H), 7.85-7.81 (m, 2H), 7.81- 7.78 (m, 1H), 2.55-2.51 (m, 3H), 1.38 (s, 6H), 1.20 (s, 6H). |
| 61 | 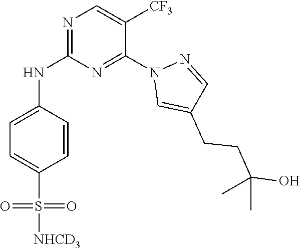 | MS (ES-API positive): 488.0 (M + H)⁺. ¹H NMR (400 MHz, DMSO-d₆) δ 10.82 (s, 1H), 9.00 (s, 1H), 8.42 (s, 1H), 8.01 (s, 1H), 7.99 (s, 1H), 7.90 (s, 1H), 7.83 (d, J = 2.0 Hz, 1H), 7.81 (d, J = 2.0 Hz, 1H), 7.35 (s, 1H), 4.34 (s, 1H), 2.66-2.58 (m, 2H), 1.78-1.68 (m, 2H), 1.19 (s, 6H). |
| 62 | 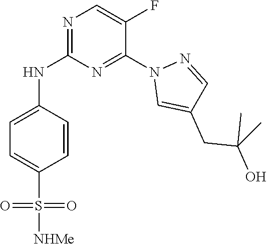 [SMILES: CNS(=O)(=O)c1ccc(Nc2ncc(F)c(-n3cc(CC(C)(C)O)cn3)n2)cc1] | MS (ES-API positive): 421.2 (M + H)⁺. ¹H NMR (400 MHz, CDCl₃) δ 8.48 (d, J = 3.9 Hz, 1H), 8.30 (s, 1H), 7.87-7.76 (m, 5H), 7.50 (s, 1H), 2.73 (s, 2H), 2.68 (d, J = 5.4 Hz, 3H), 1.29 (s, 6H). |
| 63 | 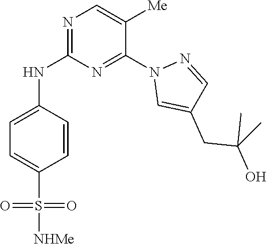 [SMILES: CNS(=O)(=O)c1ccc(Nc2ncc(C)c(-n3cc(CC(C)(C)O)cn3)n2)cc1] | MS (ES-API positive): 417.2 (M + H)⁺. ¹H NMR (400 MHz, DMSO-d₆) δ 10.15 (s, 1H), 8.51 (s, 1H), 8.45 (s, 1H), 7.98-7.93 (m, 2H), 7.76 (s, 1H), 7.73-7.69 (m, 2H), 7.25 (q, J = 5.1 Hz, 1H), 4.44 (d, J = 6.8 Hz, 1H), 2.62 (s, 2H), 2.51 (d, J = 2.0 Hz, 3H), 2.40 (d, J = 5.1 Hz, 3H), 1.12 (s, 6H). |
| 64 | 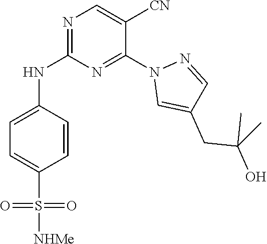 [SMILES: CNS(=O)(=O)c1ccc(Nc2ncc(C#N)c(-n3cc(CC(C)(C)O)cn3)n2)cc1] | MS (ES-API positive): 428.1 (M + H)⁺. ¹H NMR (400 MHz, DMSO-d₆) δ 8.96-8.80 (m, 1H), 8.58 (d, J = 2.7 Hz, 1H), 7.97 (d, J = 8.7 Hz, 2H), 7.88-7.75 (m, 4H), 7.54-7.39 (m, 1H), 4.06 (t, J = 7.6 Hz, 1H), 4.00- 3.93 (m, 1H), 3.92-3.84 (m, 1H), 3.59 (t, J = 7.6 Hz, 1H), 3.44-3.37 (m, 1H), 2.54 (s, 3H), 0.95-0.83 (m, 2H). |
| 65 | 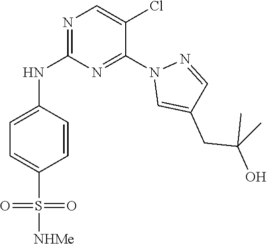 [SMILES: CNS(=O)(=O)c1ccc(Nc2ncc(Cl)c(-n3cc(CC(C)(C)O)cn3)n2)cc1] | MS (ES-API positive): 437.1 (M + H)⁺. ¹H NMR (400 MHz, DMSO-d₆) δ 10.48 (s, 1H), 8.73 (s, 1H), 8.36 (s, 1H), 7.96-7.91 (m, 2H), 7.79 (s, 1H), 7.72 (d, J = 8.7 Hz, 2H), 7.29 (d, J = 5.1 Hz, 1H), 2.61 (s, 2H), 2.40-2.37 (m, 3H), 1.11 (s, 6H). |
| 66 | 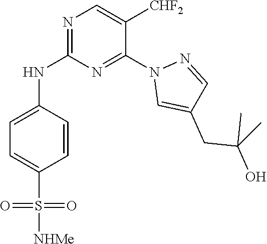 [SMILES: CNS(=O)(=O)c1ccc(Nc2ncc(C(F)F)c(-n3cc(CC(C)(C)O)cn3)n2)cc1] | MS (ES-API positive): 453.14 (M + H)⁺. ¹H NMR (500 MHz, Methanol-d₄) δ 8.84 (s, 1H), 8.53 (s, 1H), 7.96 (d, J = 8.9 Hz, 2H), 7.84-7.79 (m, 2H), 7.77 (s, 1H), 2.71 (s, 2H), 2.54 (s, 3H), 1.24 (s, 6H). |
| 67 | 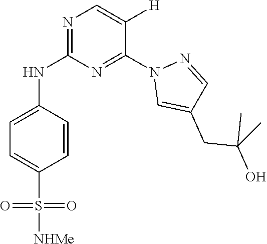 [SMILES: CNS(=O)(=O)c1ccc(Nc2nccc(-n3cc(CC(C)(C)O)cn3)n2)cc1] | MS (ES-API positive): 403.1 (M + H)⁺. ¹H NMR (400 MHz, Methanol-d₄) δ 8.54 (d, J = 5.6 Hz, 1H), 8.49 (s, 1H), 7.99-7.94 (m, 2H), 7.85-7.81 (m, 2H), 7.76 (s, 1H), 7.40 (d, J = 5.6 Hz, 1H), 2.73 (s, 2H), 2.56 (s, 3H), 1.25 (s, 6H). |
| 68 | 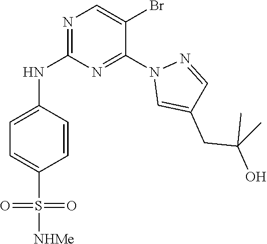 [SMILES: CNS(=O)(=O)c1ccc(Nc2ncc(Br)c(-n3cc(CC(C)(C)O)cn3)n2)cc1] | MS (ES-API positive): 481 & 483.0 (M + H)⁺. ¹H NMR (400 MHz, Methanol-d₄) δ 8.84-8.60 (m, 1H), 8.36 (s, 1H), 7.97-7.88 (m, 2H), 7.84-7.73 (m, 3H), 2.76-2.69 (m, 2H), 2.53 (s, 3H), 1.29-1.19 (m, 7H). |
| 69 | 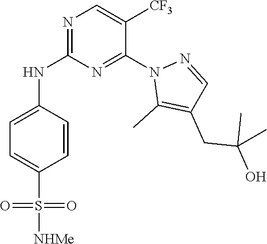 [SMILES: CNS(=O)(=O)c1ccc(Nc2ncc(C(F)(F)F)c(-n3ncc(CC(C)(C)O)c3C)n2)cc1] | MS (ES-API positive): 485.2 (M + H)⁺. ¹H NMR (400 MHz, Methanol-d₄) δ 8.94 (s, 1H), 7.97- 7.92 (m, 2H), 7.79 (d, J = 8.8 Hz, 2H), 7.61 (s, 1H), 2.64 (s, 2H), 2.52 (s, 3H), 2.40 (s, 3H), 1.22 (s, 6H). |
| 70 | 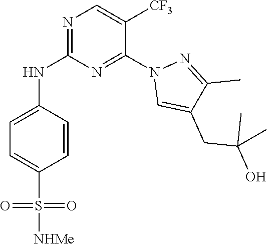 [SMILES: CNS(=O)(=O)c1ccc(Nc2ncc(C(F)(F)F)c(-n3cc(CC(C)(C)O)c(C)n3)n2)cc1] | MS (ES-API positive): 485.0 (M + H)⁺. ¹H NMR (400 MHz, Methanol-d₄) δ 8.83 (s, 1H), 8.38 (s, 1H), 7.97 (d, J = 8.8 Hz, 2H), 7.82 (d, J = 8.9 Hz, 2H), 2.66 (s, 2H), 2.54 (s, 3H), 2.30 (s, 3H), 1.26 (s, 6H). |
| 71 | 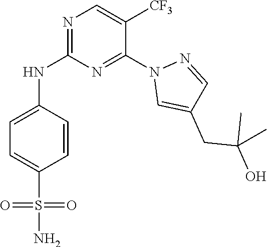 [SMILES: CC(C)(O)Cc1cnn(-c2nc(Nc3ccc(S(N)(=O)=O)cc3)ncc2C(F)(F)F)c1] | MS (ES-API positive): 457.2 (M + H)⁺. ¹H NMR (400 MHz, DMSO-d₆) δ 10.76 (s, 1H), 8.95 (s, 1H), 8.39 (s, 1H), 7.93 (s, 1H), 7.91 (s, 1H), 7.85-7.80 (m, 2H), 7.80-7.78 (m, 1H), 7.27 (s, 2H), 4.47 (s, 1H), 2.61 (s, 2H), 1.11 (s, 6H). |
| 72 | 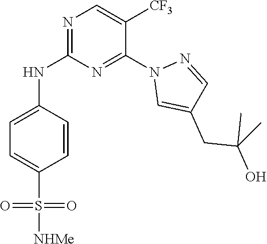 [SMILES: CNS(=O)(=O)c1ccc(Nc2ncc(C(F)(F)F)c(-n3cc(CC(C)(C)O)cn3)n2)cc1] | MS (ES-API positive): 470.1 (M + H)⁺. ¹H NMR (400 MHz, Methanol-d₄) δ 8.91 (s, 1H), 8.51 (s, 1H), 8.00-7.89 (m, 2H), 7.87-7.76 (m, 3H), 2.91 (s, 2H), 2.54 (s, 3H), 1.40 (s, 6H). |
| 73 | 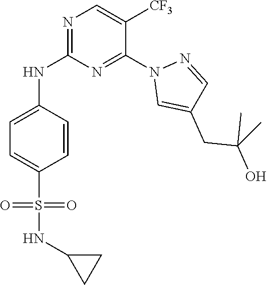 [SMILES: CC(C)(O)Cc1cnn(-c2nc(Nc3ccc(S(=O)(=O)NC4CC4)cc3)ncc2C(F)(F)F)c1] | MS (ES-API positive): 497.2 (M + H)⁺. ¹H NMR (400 MHz, DMSO-d₆) δ 8.96 (s, 1H), 8.40 (s, 1H), 7.98 (s, 1H), 7.96 (s, 1H), 7.85-7.73 (m, 4H), 4.46 (s, 1H), 2.61 (s, 2H), 2.15-2.06 (m, 1H), 1.11 (s, 6H), 0.55-0.44 (m, 2H), 0.41-0.33 (m, 2H). |
| 74 | 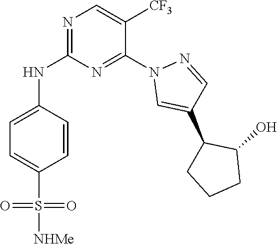 [SMILES: CNS(=O)(=O)c1ccc(Nc2ncc(C(F)(F)F)c(-n3cc([C@@H]4CCC[C@H]4O)cn3)n2)cc1] | MS (ES-API positive): 483.1 (M + H)⁺. ¹H NMR (400 MHz, Methanol-d₄) δ 8.87 (s, 1H), 8.43 (s, 1H), 7.97 (d, J = 8.8 Hz, 2H), 7.89-7.76 (m, 3H), 4.07 (q, J = 6.8 Hz, 1H), 2.92 (q, J = 7.8 Hz, 1H), 2.55 (s, 3H), 2.30-2.17 (m, 1H), 2.13-2.00 (m, 1H), 1.95-1.63 (m, 4H). |
| | Isomer-1 | |
| 75 | 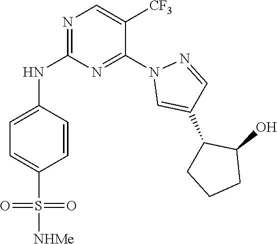 [SMILES: CNS(=O)(=O)c1ccc(Nc2ncc(C(F)(F)F)c(-n3cc([C@H]4CCC[C@@H]4O)cn3)n2)cc1] | MS (ES-API positive): 483.1 (M + H)⁺. ¹H NMR (400 MHz, Methanol-d₄) δ 8.86 (s, 1H), 8.42 (s, 1H), 7.96 (d, J = 8.9 Hz, 2H), 7.87-7.74 (m, 3H), 4.07 (q, J = 6.8 Hz, 1H), 2.92 (q, J = 7.8 Hz, 1H), 2.26-2.14 (m, 1H), 2.13-2.01 (m, 1H), 1.93-1.62 (m, 4H). |
| | Isomer-2 | |
| 76 | 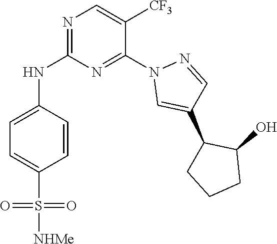 [SMILES: CNS(=O)(=O)c1ccc(Nc2ncc(C(F)(F)F)c(-n3cc([C@@H]4CCC[C@@H]4O)cn3)n2)cc1] | MS (ES-API positive): 483.2 (M + H)⁺. ¹H NMR (400 MHz, Methanol-d₄) δ 8.76 (s, 1H), 8.35 (s, 1H), 7.87 (d, J = 8.8 Hz, 2H), 7.75-7.70 (m, 2H), 7.69 (s, 1H), 4.19-4.12 (m, 1H), 2.95-2.83 (m, 1H), 2.02- 1.79 (m, 4H), 1.76-1.59 (m, 2H). |
| | Isomer-1 | |
| 77 | 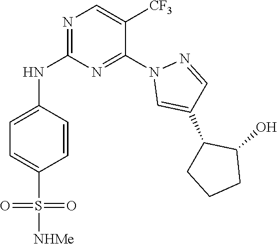 [SMILES: CNS(=O)(=O)c1ccc(Nc2ncc(C(F)(F)F)c(-n3cc([C@H]4CCC[C@H]4O)cn3)n2)cc1] | MS (ES-API positive): 483.2 (M + H)⁺. ¹H NMR (400 MHz, Methanol-d₄) δ 8.88 (s, 1H), 8.47 (s, 1H), 7.99 (d, J = 8.7 Hz, 2H), 7.87-7.83 (m, 2H), 7.81 (s, 1H), 4.27 (s, 1H), 3.07-2.98 (m, 1H), 2.13-1.92 (m, 4H), 1.87-1.73 (m, 2H). |
| | Isomer-2 | |
| 78 | 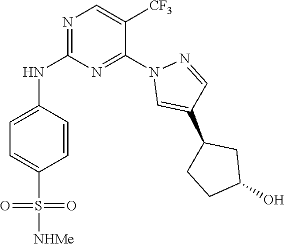 [SMILES: CNS(=O)(=O)c1ccc(Nc2ncc(C(F)(F)F)c(-n3cc([C@@H]4CC[C@@H](O)C4)cn3)n2)cc1] | MS (ES-API positive): 483.1 (M + H)⁺. ¹H NMR (400 MHz, Methanol-d₄) δ 8.86 (s, 1H), 8.36 (s, 1H), 8.01-7.92 (m, 2H), 7.84-7.79 (m, 2H), 7.75 (s, 1H), 4.53-4.41 (m, 1H), 3.40-3.33 (m, 1H), 2.54 (s, 3H), 2.33-2.22 (m, 1H), 2.21-2.06 (m, 2H), 1.84-1.80 (m, 1H), 1.75-1.54 (m, 2H). |
| | Isomer-1 | |
| 79 | 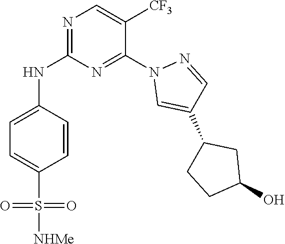 [SMILES: CNS(=O)(=O)c1ccc(Nc2ncc(C(F)(F)F)c(-n3cc([C@H]4CC[C@H](O)C4)cn3)n2)cc1] | MS (ES-API positive): 483.1 (M + H)⁺. ¹H NMR (400 MHz, Methanol-d₄) δ 8.86 (s, 1H), 8.35 (s, 1H), 7.96 (d, J = 8.8 Hz, 2H), 7.86-7.79 (m, 2H), 7.75 (s, 1H), 4.45-4.41 (m, 1H), 3.41-3.34 (m, 1H), 2.54 (s, 3H), 2.35-2.22 (m, 1H), 2.21-2.06 (m, 2H), 1.84-1.80 (m, J = 5.7, 10.4, 13.2 Hz, 1H), 1.77-1.55 (m, 2H). |
| | Isomer-2 | |
| 80 | 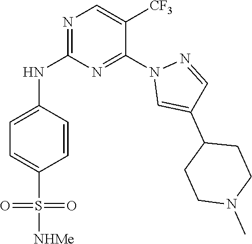 [SMILES: CNS(=O)(=O)c1ccc(Nc2ncc(C(F)(F)F)c(-n3cc(C4CCN(C)CC4)cn3)n2)cc1] | MS (ES-API positive): 496.1 (M + H)⁺. ¹H NMR (400 MHz, Methanol-d₄) δ 8.87 (s, 1H), 8.41 (s, 1H), 7.96 (d, J = 8.7 Hz, 2H), 7.83 (d, J = 7.4 Hz, 3H), 3.82 (d, J = 12.2 Hz, 2H), 2.95-2.85 (m, 5H), 2.83-2.75 (m, 1H), 2.54 (s, 3H), 2.16-2.09 (m, 2H), 1.83-1.67 (m, 2H). |
| 81 | 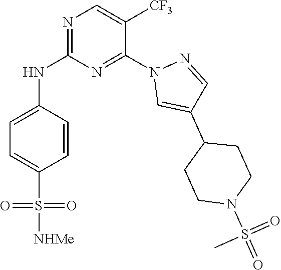 [SMILES: CNS(=O)(=O)c1ccc(Nc2ncc(C(F)(F)F)c(-n3cc(C4CCN(S(C)(=O)=O)CC4)cn3)n2)cc1] | MS (ES-API positive): 560.1 (M + H)⁺. ¹H NMR (400 MHz, Methanol-d₄) δ 8.89 (s, 1H), 8.43 (s, 1H), 8.00-7.90 (m, 2H), 7.87-7.77 (m, 3H), 3.63 (d, J = 12.6 Hz, 2H), 3.22-3.12 (m, 2H), 3.06-2.96 (m, 1H), 2.93 (s, 3H), 2.54 (s, 3H), 2.33 (d, J = 13.6 Hz, 2H), 1.96- 1.80 (m, 2H). |
| 82 | 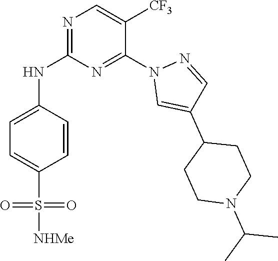 [SMILES: CNS(=O)(=O)c1ccc(Nc2ncc(C(F)(F)F)c(-n3cc(C4CCN(C(C)C)CC4)cn3)n2)cc1] | MS (ES-API positive): 524.2 (M + H)⁺. ¹H NMR (400 MHz, Methanol-d₄) δ 8.89 (s, 1H), 8.43 (s, 1H), 8.00-7.90 (m, 2H), 7.87-7.77 (m, 3H), 3.63 (d, J = 12.6 Hz, 2H), 3.22-3.12 (m, 2H), 3.06-2.96 (m, 1H), 2.93 (s, 3H), 2.54 (s, 3H), 2.33 (d, J = 13.6 Hz, 2H), 1.96- 1.80 (m, 2H). |
| 83 | 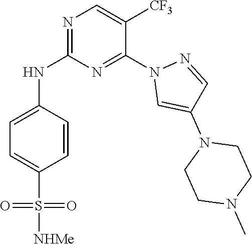 [SMILES: CNS(=O)(=O)c1ccc(Nc2ncc(C(F)(F)F)c(-n3cc(N4CCN(C)CC4)cn3)n2)cc1] | MS (ES-API positive): 497.1 (M + H)⁺. ¹H NMR (400 MHz, Methanol-d₄) δ 8.86 (s, 1H), 8.15 (s, 1H), 7.97 (d, J = 8.9 Hz, 2H), 7.87-7.81 (m, 3H), 3.37 (s, 8H), 3.00 (s, 4H), 2.56 (s, 3H). |
| 84 | 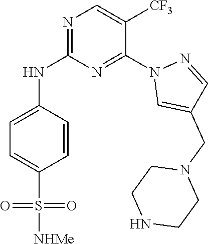 [SMILES: CNS(=O)(=O)c1ccc(Nc2ncc(C(F)(F)F)c(-n3cc(CN4CCNCC4)cn3)n2)cc1] | MS (ES-API positive): 497.1 (M + H)⁺. ¹H NMR (400 MHz, Methanol-d₄) δ 9.00 (s, 1 H), 8.94 (s, 1 H), 8.08 (s, 1 H), 8.00-7.94 (m, 2 H), 7.92-7.82 (m, 2 H), 4.58 (s, 2 H), 3.67 (s, 8 H), 2.58-2.52 (m, 3 H). |
| 85 | 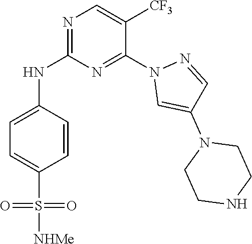 [SMILES: CNS(=O)(=O)c1ccc(Nc2ncc(C(F)(F)F)c(-n3cc(N4CCNCC4)cn3)n2)cc1] | MS (ES-API positive): 483.1 (M + H)⁺. ¹H NMR (400 MHz, Methanol-d₄) δ 8.86 (s, 1H), 8.14 (s, 1H), 7.99-7.95 (m, 2H), 7.86-7.82 (m, 3H), 3.44- 3.41 (m, 4H), 3.38-3.34 (m, 4H), 2.56 (s, 3H). |
| 86 | 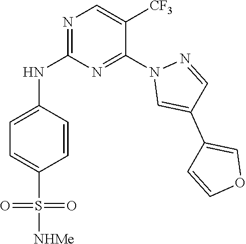 [SMILES: CNS(=O)(=O)c1ccc(Nc2ncc(C(F)(F)F)c(-n3cc(-c4ccoc4)cn3)n2)cc1] | MS (ES-API positive): 465.1 (M + H)⁺. ¹H NMR (500 MHz, Methanol-d₄) δ 8.90 (s, 1H), 8.70 (s, 1H), 8.06 (s, 1H), 7.98 (d, J = 8.9 Hz, 2H), 7.90 (s, 1H), 7.84 (d, J = 8.7 Hz, 2H), 7.60-7.57 (m, 1H), 6.75 (d, J = 0.9 Hz, 1H), 2.54 (s, 3H). |
| 87 | 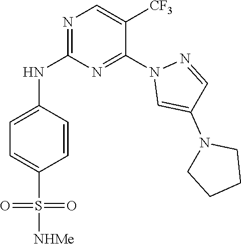 [SMILES: CNS(=O)(=O)c1ccc(Nc2ncc(C(F)(F)F)c(-n3cc(N4CCCC4)cn3)n2)cc1] | MS (ES-API positive): 468.0 (M + H)⁺. ¹H NMR (400 MHz, Methanol-d₄) δ 8.81 (s, 1H), 8.00- 7.95 (m, 3H), 7.83 (d, J=8.8 Hz, 2H), 7.69 (s, 1H), 3.29 (t, J = 6.4 Hz, 4H), 2.56 (s, 3H), 2.11-2.04 (m, 4H). |
| 88 | 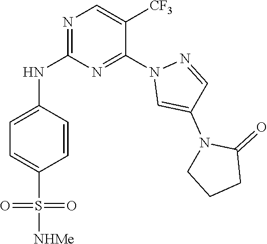 [SMILES: CNS(=O)(=O)c1ccc(Nc2ncc(C(F)(F)F)c(-n3cc(N4CCCC4=O)cn3)n2)cc1] | MS (ES-API positive): 482.0 (M + H)⁺. ¹H NMR (400 MHz, Methanol-d₄) δ 10.87 (s, 1H), 8.98 (s, 1H), 8.89 (s, 1H), 8.20 (s, 1H), 8.03-7.94 (m, 2H), 7.76 (d, J = 8.7 Hz, 2H), 7.35 (q, J = 5.0 Hz, 1H), 3.82 (t, J = 7.1 Hz, 2H), 2.49-2.46 (m, 2H), 2.44-2.38 (m, 3H), 2.15 (q, J = 7.6 Hz, 2H). |
| 89 | 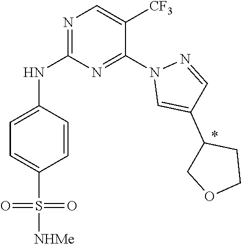 [SMILES: CNS(=O)(=O)c1ccc(Nc2ncc(C(F)(F)F)c(-n3cc(C4CCOC4)cn3)n2)cc1] | MS (ES-API positive): 469.1 (M + H)⁺. ¹H NMR (400 MHz, Methanol-d₄) δ 8.88 (s, 1H), 8.45 (s, 1H), 7.97 (d, J = 8.8 Hz, 2H), 7.85-7.66 (m, 3H), 6.47- 6.40 (m, 1H), 4.11 (dd, J = 7.1, 8.0 Hz, 1H), 4.06-3.98 (m, 1H), 3.93 (q, J = 7.6 Hz, 1H), 3.71 (dd, J = 6.7, 8.1 Hz, 1H), 3.49 (t, J = 7.2 Hz, 1H), 2.54 (s, 3H), 2.49-2.34 (m, 1H), 2.08-1.96 (m, 1H). |
| | Isomer-1 | |
| 90 | 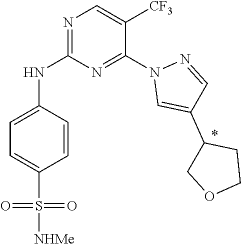 [SMILES: CNS(=O)(=O)c1ccc(Nc2ncc(C(F)(F)F)c(-n3cc(C4CCOC4)cn3)n2)cc1] | MS (ES-API positive): 469.1 (M + H)⁺. ¹H NMR (400 MHz, Methanol-d₄) δ 8.88 (s, 1H), 8.44 (s, 1H), 7.96 (d, J = 8.8 Hz, 2H), 7.89-7.66 (m, 3H), 4.10 (dd, J = 7.0, 8.1 Hz, 1H), 4.03 (d, J = 5.4 Hz, 1H), 3.94 (s, 1H), 3.71 (dd, J = 6.7, 8.2 Hz, 1H), 3.49 (t, J = 7.2 Hz, 1H), 2.54 (s, 3H), 2.47-2.34 (m, 1H), 2.07-1.94 (m, 1H). |
| | Isomer-2 | |
| 91 | 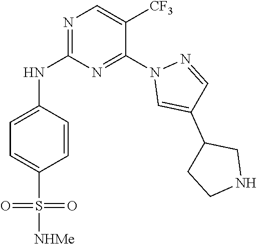 [SMILES: CNS(=O)(=O)c1ccc(Nc2ncc(C(F)(F)F)c(-n3cc(C4CCNC4)cn3)n2)cc1] | MS (ES-API positive): 468.1 (M + H)⁺. ¹H NMR (400 MHz, Methanol-d₄) δ 8.99-8.94 (m, 1H), 8.75-8.72 (m, 1H), 8.05-8.02 (m, 1H), 7.98-7.94 (m, 2H), 7.88-7.83 (m, 2H), 4.81-4.76 (m, 1H), 3.52-3.42 (m, 2H), 2.61-2.52 (m, 4H), 2.39-2.20 (m, 3H). |
| | Isomer-1 | |
| 92 | 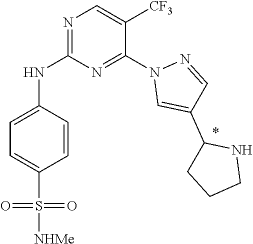 [SMILES: CNS(=O)(=O)c1ccc(Nc2ncc(C(F)(F)F)c(-n3cc(C4CCCN4)cn3)n2)cc1] | MS (ES-API positive): 468.1 (M + H)⁺. ¹H NMR (400 MHz, Methanol-d₄) δ 8.96 (s, 1H), 8.79- 8.70 (m, 1H), 8.03-8.02 (m, 1H), 7.98-7.95 (m, 2H), 7.88-7.83 (m, 2H), 4.82-4.77 (m, 1H), 3.53-3.42 (m, 2H), 2.60-2.53 (m, 4H), 2.37-2.21 (m, 3H). |
| | Isomer-2 | |
| 93 | 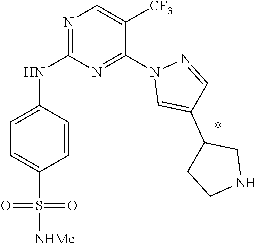 [SMILES: CNS(=O)(=O)c1ccc(Nc2ncc(C(F)(F)F)c(-n3cc(C4CCNC4)cn3)n2)cc1] | MS (ES-API positive): 468.1 (M + H)⁺. ¹H NMR (400 MHz, Methanol-d₄) δ 8.92 (s, 1H), 8.55 (s, 1H), 7.97 (d, J = 8.8 Hz, 2H), 7.91 (s, 1H), 7.85 (d, J = 8.8 Hz, 2H), 3.76 (dd, J = 7.9, 11.2 Hz, 1H), 3.70-3.53 (m, 2H), 3.48-3.39 (m, 1H), 3.25 (dd, J = 9.5, 11.1 Hz, 1H), 2.56 (s, 4H), 2.23-2.11 (m, 1H). |
| | Isomer-1 | |
| 94 | 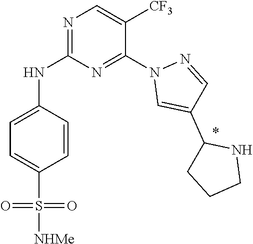 [SMILES: CNS(=O)(=O)c1ccc(Nc2ncc(C(F)(F)F)c(-n3cc(C4CCCN4)cn3)n2)cc1] | MS (ES-API positive): 468.1 (M + H)⁺. ¹H NMR (400 MHz, Methanol-d₄) δ 8.96-8.83 (m, 1H), 8.55 (s, 1H), 7.97 (d, J = 8.8 Hz, 2H), 7.91 (s, 1H), 7.85 (d, J = 8.8 Hz, 2H), 3.83-3.71 (m, 1H), 3.68-3.53 (m, 2H), 3.47-3.40 (m, 1H), 3.25 (dd, J = 9.7, 11.0 Hz, 1H), 2.56 (s, 4H), 2.25-2.06 (m, 1H). |
| | Isomer-2 | |
| 95 | 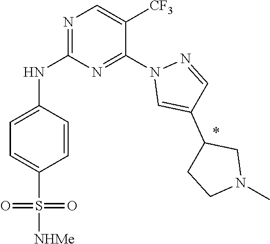 [SMILES: CNS(=O)(=O)c1ccc(Nc2ncc(C(F)(F)F)c(-n3cc(C4CCN(C)C4)cn3)n2)cc1] | MS (ES-API positive): 482.2 (M + H)⁺. ¹H NMR (400 MHz, Methanol-d₄) δ 8.92 (s, 1H), 8.55 (s, 1H), 7.97 (d, J = 8.8 Hz, 2H), 7.94-7.89 (m, 1H), 7.85 (d, J = 8.8 Hz, 2H), 3.99 (d, J = 3.0 Hz, 1H), 3.97-3.75 (m, 2H), 3.73-3.52 (m, 2H), 3.28-3.12 (m, 1H), 3.05 (s, 3H), 2.56 (s, 3H), 2.39-2.13 (m, 1H). |
| | Isomer-1 | |
| 96 | 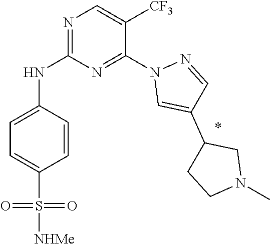 [SMILES: CNS(=O)(=O)c1ccc(Nc2ncc(C(F)(F)F)c(-n3cc(C4CCN(C)C4)cn3)n2)cc1] | MS (ES-API positive): 482.2 (M + H)⁺. ¹H NMR (400 MHz, Methanol-d₄) δ 8.92 (s, 1H), 8.55 (s, 1H), 7.97 (d, J=8.8 Hz, 2H), 7.93-7.88 (m, 1H), 7.85 (d, J = 8.8 Hz, 2H), 4.12-3.97 (m, 1H), 3.94-3.78 (m, 1H), 3.75-3.55 (m, 2H), 3.05 (s, 3H), 2.56 (s, 3H), 2.44-2.13 (m, 1H). |
| | Isomer-2 | |
| 97 | 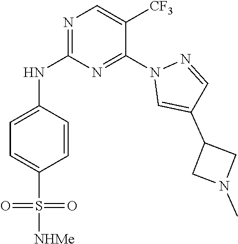 [SMILES: CNS(=O)(=O)c1ccc(Nc2ncc(C(F)(F)F)c(-n3cc(C4CN(C)C4)cn3)n2)cc1] | MS (ES-API positive): 468.0 (M + H)⁺. ¹H NMR (400 MHz, Methanol-d₄) δ 8.93 (s, 1H), 8.62 (s, 1H), 8.03 (s, 1H), 7.96 (s, 2H), 7.85 (d, J = 8.8 Hz, 2H), 4.69-4.59 (m, 1H), 4.54-4.33 (m, 2H), 4.30-4.11 (m, 2H), 3.05 (s, 3H), 2.56 (s, 3H). |
| 98 | 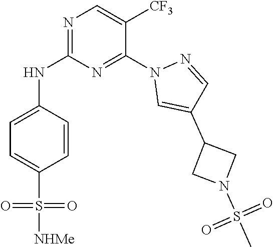 [SMILES: CNS(=O)(=O)c1ccc(Nc2ncc(C(F)(F)F)c(-n3cc(C4CN(S(C)(=O)=O)C4)cn3)n2)cc1] | MS (ES-API positive): 532.0 (M + H)⁺. ¹H NMR (400 MHz, Methanol-d₄) δ 8.91 (s, 1H), 8.63 (s, 1H), 7.98 (d, J = 8.9 Hz, 2H), 7.93 (s, 1H), 7.85 (d, J = 8.9 Hz, 2H), 4.37-4.24 (m, 2H), 4.05-3.96 (m, 2H), 3.96-3.88 (m, 1H), 3.03 (s, 3H), 2.56 (s, 3H) |
| 99 | 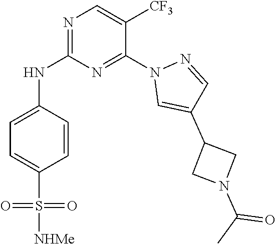 [SMILES: CNS(=O)(=O)c1ccc(Nc2ncc(C(F)(F)F)c(-n3cc(C4CN(C(C)=O)C4)cn3)n2)cc1] | MS (ES-API positive): 496.1 (M + H)⁺. ¹H NMR (400 MHz, Methanol-d₄) δ 8.91 (s, 1H), 8.63 (s, 1H), 7.98 (d, J = 8.9 Hz, 2H), 7.93 (s, 1H), 7.85 (d, J = 8.9 Hz, 2H), 4.37-4.24 (m, 2H), 4.05-3.96 (m, 2H), 3.96-3.88 (m, 1H), 3.03 (s, 3H), 2.56 (s, 3H). |
| 100 | 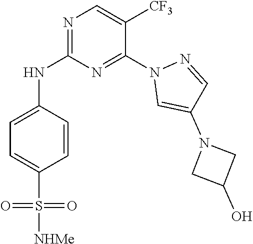 [SMILES: CNS(=O)(=O)c1ccc(Nc2ncc(C(F)(F)F)c(-n3cc(N4CC(O)C4)cn3)n2)cc1] | MS (ES-API positive): 470.1 (M + H)⁺. ¹H NMR (400 MHz, Methanol-d₄) δ 8.83 (s, 1H), 7.98 (d, J = 8.8 Hz, 2H), 7.93 (s, 1H), 7.86-7.80 (m, 2H), 7.54 (d, J = 0.7 Hz, 1H), 4.72-4.64 (m, 1H), 4.15 (dd, J = 6.5, 8.0 Hz, 2H), 3.63-3.56 (m, 2H), 2.56 (s, 3H). |
| 101 | 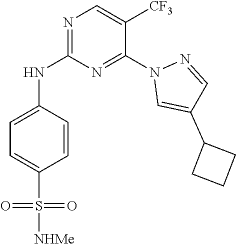 [SMILES: CNS(=O)(=O)c1ccc(Nc2ncc(C(F)(F)F)c(-n3cc(C4CCC4)cn3)n2)cc1] | MS (ES-API positive): 453.1 (M + H)⁺. ¹H NMR (400 MHz, Methanol-d₄) δ 8.87 (s, 1H), 8.36 (s, 1H), 7.97 (d, J = 8.8 Hz, 2H), 7.82 (d, J = 8.7 Hz, 2H), 7.77 (s, 1H), 3.54 (t, J = 8.3 Hz, 1H), 2.54 (s, 3H), 2.46- 2.36 (m, 2H), 2.19-2.10 (m, 2H), 2.09-1.93 (m, 2H). |
| 102 | 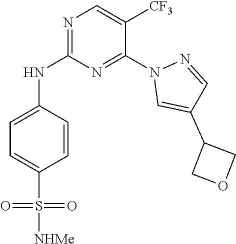 [SMILES: CNS(=O)(=O)c1ccc(Nc2ncc(C(F)(F)F)c(-n3cc(C4COC4)cn3)n2)cc1] | MS (ES-API positive): 455.0 (M + H)⁺. ¹H NMR (400 MHz, Methanol-d₄) δ 8.89 (s, 1H), 8.57 (s, 1H), 7.99-7.93 (m, 3H), 7.83 (d, J = 8.8 Hz, 2H), 5.07 (dd, J = 5.9, 8.3 Hz, 2H), 4.75 (t, J = 6.4 Hz, 2H), 4.34 (s, 1H), 2.54 (s, 3H). |
| 103 | 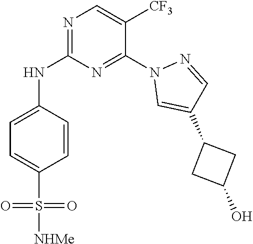 [SMILES: CNS(=O)(=O)c1ccc(Nc2ncc(C(F)(F)F)c(-n3cc([C@H]4C[C@@H](O)C4)cn3)n2)cc1] | MS (ES-API positive): 469.0 (M + H)⁺ ¹H NMR (400 MHz, Methanol-d₄) δ 8.87 (s, 1H), 8.36 (s, 1H), 7.96 (d, J = 8.7 Hz, 2H), 7.82 (d, J = 8.8 Hz, 2H), 7.77 (s, 1H), 4.19 (q, J = 7.5 Hz, 1H), 3.02-2.90 (m, 1H), 2.80-2.69 (m, 2H), 2.54 (s, 3H), 2.05-1.94 (m, 2H). |
| 104 | 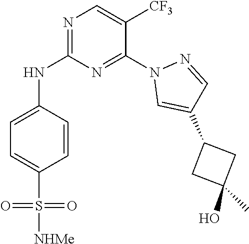 [SMILES: CNS(=O)(=O)c1ccc(Nc2ncc(C(F)(F)F)c(-n3cc([C@H]4C[C@](C)(O)C4)cn3)n2)cc1] | MS (ES-API positive): 483.1 (M + H)⁺. ¹H NMR (400 MHz, Methanol-d₄) δ 8.86 (s, 1H), 8.37 (s, 1H), 7.96 (d, J = 8.9 Hz, 2H), 7.87-7.79 (m, 2H), 7.75 (s, 1H), 3.58 (q, J = 8.2 Hz, 1H), 2.58-2.50 (m, 5H), 2.22- 2.13 (m, 2H), 1.35 (s, 3H). |
| | Isomer-1 | |
| 105 | 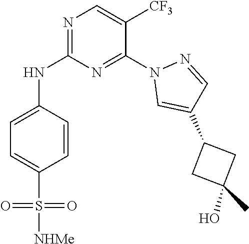 [SMILES: CNS(=O)(=O)c1ccc(Nc2ncc(C(F)(F)F)c(-n3cc([C@H]4C[C@@](C)(O)C4)cn3)n2)cc1] | MS (ES-API positive): 483.1 (M + H)⁺. ¹H NMR (400 MHz, Methanol-d₄) δ 8.86 (s, 1H), 8.36 (s, 1H), 7.96 (d, J = 8.9 Hz, 2H), 7.87-7.81 (m, 2H), 7.79 (s, 1H), 3.06 (q, J = 8.9 Hz, 1H), 2.54 (s, 3H), 2.51-2.45 (m, 2H), 2.23-2.14 (m, 2H), 1.44 (s, 3H). |
| | Isomer-2 | |
| 106 | 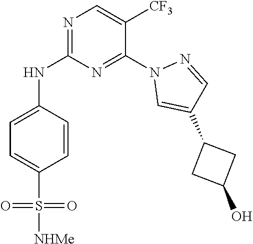 [SMILES: CNS(=O)(=O)c1ccc(Nc2ncc(C(F)(F)F)c(-n3cc([C@H]4C[C@H](O)C4)cn3)n2)cc1] | MS (ES-API positive):469.1 (M + H)⁺. ¹H NMR (400 MHz, Methanol-d₄) δ 8.88 (s, 1H), 8.42 (s, 1H), 7.98 (d, J = 8.8 Hz, 2H), 7.88-7.72 (m, 3H), 4.62- 4.42 (m, 1H), 3.55-3.41 (m, 1H), 2.56 (s, 3H), 2.48-2.37 (m, 4H). |
| 107 | 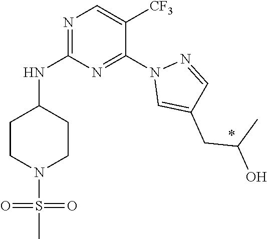 [SMILES: CC(O)Cc1cnn(-c2nc(NC3CCN(S(C)(=O)=O)CC3)ncc2C(F)(F)F)c1] | MS (ES-API positive): 449.0 (M + H)⁺. ¹H NMR (400 MHz, Methanol-d₄) δ 8.63 (d, J = 19.4 Hz, 1H), 8.35 (d, J = 7.5 Hz, 1H), 7.71 (d, J = 6.4 Hz, 1H), 4.13-4.04 (m, 1H), 3.97 (s, 1H), 3.75 (d, J = 12.4 Hz, 2H), 2.99 (dd, J = 7.3, 9.4 Hz, 2H), 2.89 (s, 3H), 2.68 (d, J = 6.0 Hz, 2H), 2.19-2.09 (m, 2H), 1.72 (d, J = 9.9 Hz, 2H), 1.22 (s, 3H). |
| | Isomer-1 | |
| 108 | 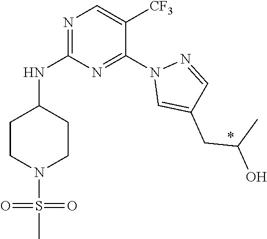 [SMILES: CC(O)Cc1cnn(-c2nc(NC3CCN(S(C)(=O)=O)CC3)ncc2C(F)(F)F)c1] | MS (ES-API positive): 449.0 (M + H)⁺. ¹H NMR (400 MHz, Methanol-d₄) δ 8.63 (d, J = 19.4 Hz, 1H), 8.35 (d, J = 7.5 Hz, 1H), 7.71 (d, J = 6.4 Hz, 1H), 4.13-4.04 (m, 1H), 3.97 (s, 1H), 3.75 (d, J = 12.4 Hz, 2H), 2.99 (dd, J = 7.3, 9.4 Hz, 2H), 2.89 (s, 3H), 2.68 (d, J = 6.0 Hz, 2H), 2.19-2.09 (m, 2H), 1.72 (d, J = 9.9 Hz, 2H), 1.22 (s, 3H). |
| | Isomer-2 | |
| 109 | 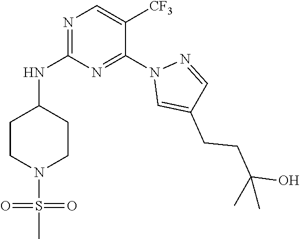 [SMILES: CC(C)(O)CCc1cnn(-c2nc(NC3CCN(S(C)(=O)=O)CC3)ncc2C(F)(F)F)c1] | MS (ES-API positive): 477.2 (M + H)⁺. ¹H NMR (400 MHz, Methanol-d₄) δ 8.63 (dd, J = 3.3, 15.9 Hz, 1H), 8.29 (s, 1H), 7.70 (d, J = 3.3 Hz, 1H), 4.17- 4.00 (m, 1H), 3.75 (d, J = 12.2 Hz, 2H), 3.07-2.94 (m, 2H), 2.89 (s, 3H), 2.71-2.60 (m, 2H), 2.14 (d, J = 10.1 Hz, 2H), 1.84-1.64 (m, 4H), 1.28 (s, 6H). |
| 110 | 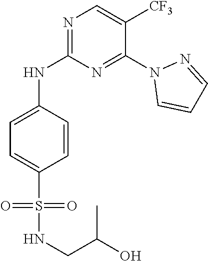 [SMILES: CC(O)CNS(=O)(=O)c1ccc(Nc2ncc(C(F)(F)F)c(-n3cccn3)n2)cc1] | MS (ES-API positive): 443.0 (M + H)⁺. ¹H NMR (400 MHz, DMSO-d₆) δ 10.85 (s, 1H), 9.00 (s, 1H), 8.63-8.59 (m, 1H), 7.98 (dd, J = 1.6, 0.7 Hz, 1H), 7.94 (d, J = 8.8 Hz, 2H), 7.79 (d, J = 8.9 Hz, 2H), 7.45 (s, 1H), 6.70 (dd, J = 2.8, 1.6 Hz, 1H), 4.68 (d, J = 4.7 Hz, 1H), 3.59 (dt, J = 10.9, 6.0 Hz, 1H), 2.64 (dd, J = 15.7, 6.0 Hz, 2H), 0.99 (d, J = 6.2 Hz, 3H). |
| 111 | 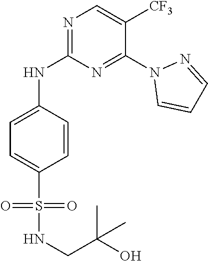 [SMILES: CC(C)(O)CNS(=O)(=O)c1ccc(Nc2ncc(C(F)(F)F)c(-n3cccn3)n2)cc1] | MS (ES-API positive): 456.8 (M + H)⁺. ¹H NMR (400 MHz, DMSO-d₆) δ 10.83 (s, 1H), 9.00 (s, 1H), 8.61 (d, J = 2.8 Hz, 1H), 7.98 (d, J = 1.7 Hz, 1H), 7.93 (d, J = 8.5 Hz, 2H), 7.80 (d, J = 8.5 Hz, 2H), 7.37 (t, J = 6.3 Hz, 1H), 6.69 (dd, J = 2.8, 1.4 Hz, 1H), 4.41 (d, J = 1.0 Hz, 1H), 2.61 (d, J = 6.0 Hz, 2H), 1.05 (s, 6H). |
| 112 | 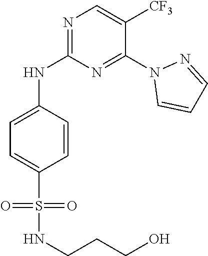 [SMILES: O=S(=O)(NCCCO)c1ccc(Nc2ncc(C(F)(F)F)c(-n3cccn3)n2)cc1] | MS (ES-API positive): 443.2 (M + H)⁺. ¹H NMR (400 MHz, DMSO-d₆) δ 10.86 (s, 1H), 9.00 (s, 1H), 8.62 (d, J = 2.8 Hz, 1H), 7.98 (d, J = 1.6 Hz, 1H), 7.94 (d, J = 8.8 Hz, 2H), 7.78 (d, J = 8.8 Hz, 2H), 7.43 (t, J = 5.9 Hz, 1H), 6.70 (dd, J = 2.8, 1.6 Hz, 1H), 4.44 (t, J = 5.1 Hz, 1H), 3.34 (s, 2H), 2.84-2.74 (m, 2H), 1.55-1.49 (m, 2H). |
| 113 | 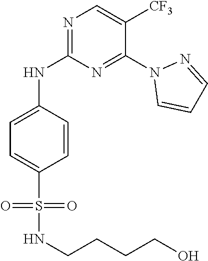 [SMILES: O=S(=O)(NCCCCO)c1ccc(Nc2ncc(C(F)(F)F)c(-n3cccn3)n2)cc1] | MS (ES-API positive): 457.0 (M + H)⁺. ¹H NMR (400 MHz, DMSO-d₆) δ 10.85 (s, 1H), 9.00 (s, 1H), 8.61 (d, J = 2.7 Hz, 1H), 7.98 (d, J = 1.6 Hz, 1H), 7.94 (d, J = 8.5 Hz, 2H), 7.78 (d, J = 8.4 Hz, 2H), 7.47 (t, J = 5.9 Hz, 1H), 6.69 (t, J = 2.1 Hz, 1H), 4.39 (t, J = 5.1 Hz, 1H), 3.32 (d, J = 5.4 Hz, 2H), 2.75-2.70 (m, 2H), 1.44-1.32 (m, 4H). |
| 114 | 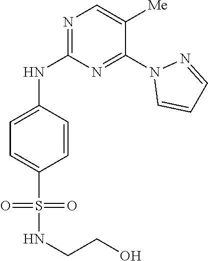 [SMILES: Cc1cnc(Nc2ccc(S(=O)(=O)NCCO)cc2)nc1-n1cccn1] | MS (ES-API positive): 375.2 (M + H)⁺. ¹H NMR (400 MHz, Methanol-d₄/CDCl₃) δ 8.60 (d, J = 2.7 Hz, 1H), 8.42 (s, 1H), 7.92-7.89 (m, 2 H), 7.82-7.81 (m, 2H), 7.80-7.79 (m, 1 H), 6.56 (t, 1H), 3.58 (t, J = 5.9 Hz, 2H), 2.99 (t, J = 5.9 Hz, 2H), 2.56 (s, 3H). |
| 115 | 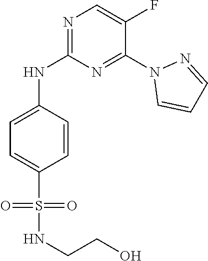 [SMILES: O=S(=O)(NCCO)c1ccc(Nc2ncc(F)c(-n3cccn3)n2)cc1] | MS (ES-API positive): 379.0 (M + H)⁺. ¹H NMR (400 MHz, DMSO-d₆) δ 10.38 (s, 1H), 8.79 (d, J = 4.0 Hz, 1H), 8.60 (d, J = 2.7 Hz, 1H), 8.00 (d, J = 1.6 Hz, 1H), 7.97-7.86 (m, 2H), 7.81-7.65 (m, 2H), 7.42 (t, J = 6.0 Hz, 1H), 6.72 (dd, J = 2.7, 1.7 Hz, 1H), 4.69 (t, J = 5.5 Hz, 1H), 3.45 (s, 2H), 2.78 (q, J = 6.3 Hz, 2H). |
| 116 | 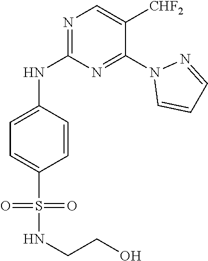 [SMILES: O=S(=O)(NCCO)c1ccc(Nc2ncc(C(F)F)c(-n3cccn3)n2)cc1] | MS (ES-API positive): 411.0 (M + H)⁺. ¹H NMR (400 MHz, DMSO-d₆) δ 10.66 (s, 1H), 8.91 (s, 1H), 8.71 (d, J = 2.7 Hz, 1H), 8.03 (d, J = 1.6 Hz, 1H), 7.95 (d, J = 8.9 Hz, 2H), 7.80 (dd, J = 9.3, 7.3 Hz, 3H), 7.46 (t, J = 5.9 Hz, 1H), 6.72 (dd, J = 2.8, 1.6 Hz, 1H), 4.70 (t, J = 5.5 Hz, 1H), 3.43-3.37 (m, 2H), 2.79 (q, J = 6.2 Hz, 2H). |
| 117 | 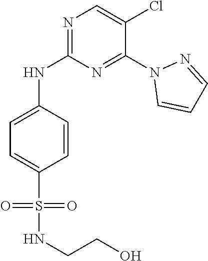 [SMILES: O=S(=O)(NCCO)c1ccc(Nc2ncc(Cl)c(-n3cccn3)n2)cc1] | MS (ES-API positive): 395.2 (M + H)⁺. ¹H NMR (400 MHz, Methanol-d₄) δ 8.62 (s, 1H), 8.54 (d, J = 2.7 Hz, 1H), 7.98-7.86 (m, 3H), 7.85- 7.78 (m, 2H), 6.62 (dd, J = 2.8, 1.7 Hz, 1H), 3.58 (t, J = 5.9 Hz, 2H), 2.98 (t, J = 5.9 Hz, 2H). |
| 118 | 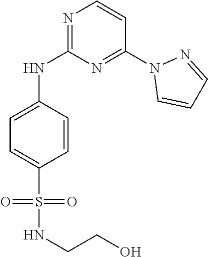 [SMILES: O=S(=O)(NCCO)c1ccc(Nc2nccc(-n3cccn3)n2)cc1] | MS (ES-API positive): 361.2 (M + H)⁺. ¹H NMR (400 MHz, Methanol-d₄) δ 8.63 (d, J = 2.8 Hz, 1H), 8.54 (d, J = 5.6 Hz, 1H), 8.06-7.89 (m, 2H), 7.86- 7.79 (m, 2H), 7.39 (d, J = 5.4 Hz, 1H), 6.59 (dd, J = 2.8, 1.6 Hz, 1H), 3.60 (t, J = 5.8 Hz, 2H), 3.01 (t, J = 5.8 Hz, 2H). |
| 119 | 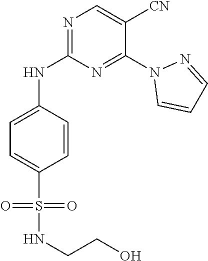 [SMILES: N#Cc1cnc(Nc2ccc(S(=O)(=O)NCCO)cc2)nc1-n1cccn1] | MS (ES-API positive): 386.2 (M + H)⁺. ¹H NMR (400 MHz, DMSO-d₆) δ 10.98 (s, 1H), 9.01 (s, 1H), 8.65 (d, J = 2.7 Hz, 1H), 8.07 (d, J = 1.6 Hz, 1H), 7.94 (d, J = 8.6 Hz, 2H), 7.82 (d, J = 8.5 Hz, 2H), 7.53 (t, J = 6.0 Hz, 1H), 6.78-6.73 (m, 1H), 4.72 (t, J = 5.6 Hz, 1H), 3.51-3.36 (m, 2H), 2.79 (q, J = 6.3 Hz, 2H). |
| 120 | 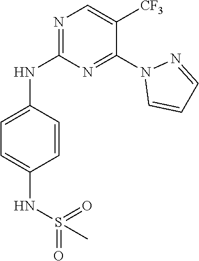 [SMILES: CS(=O)(=O)Nc1ccc(Nc2ncc(C(F)(F)F)c(-n3cccn3)n2)cc1] | MS (ES-API positive): 399.2 (M + H)⁺. ¹H NMR (400 MHz, DMSO-d₆) δ 10.54 (s, 1H), 9.87 (s, 1H), 8.92 (s, 1H), 8.64 (s, 1H), 7.96 (s, 1H), 7.72 (s, 1H), 7.39 (s, 1H), 7.31 (t, J = 8.0 Hz, 1H), 6.92 (d, J = 7.9 Hz, 1H), 6.70-6.69 (m, 1H), 3.02 (s, 3H). |
| 121 | 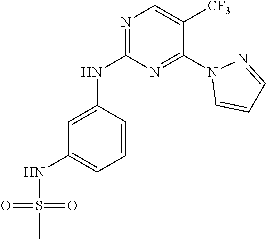 [SMILES: CS(=O)(=O)Nc1cccc(Nc2ncc(C(F)(F)F)c(-n3cccn3)n2)c1] | MS (ES-API positive): 399.2 (M + H)⁺. ¹H NMR (400 MHz, DMSO-d₆) δ 10.54 (s, 1H), 9.87 (s, 1H), 8.92 (s, 1H), 8.64 (s, 1H), 7.96 (s, 1H), 7.72 (s, 1H), 7.39 (s, 1H), 7.31 (t, J = 8.0 Hz, 1H), 6.92 (d, J = 7.9 Hz, 1H), 6.70-6.69 (m, 1H), 3.02 (s, 3H). |
| 122 | 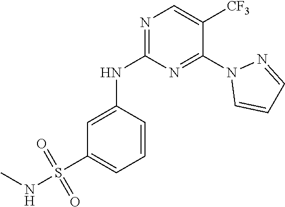 [SMILES: CNS(=O)(=O)c1cccc(Nc2ncc(C(F)(F)F)c(-n3cccn3)n2)c1] | MS (ES-API positive): 399.2 (M + H)⁺. ¹H NMR (400 MHz, DMSO-d₆) δ 10.83 (s, 1H), 8.98 (d, J = 4.5 Hz, 1H), 8.63 (s, 1H), 8.44 (s, 1H), 7.98 (d, J = 4.3 Hz, 1H), 7.84 (s, 1H), 7.63-7.58 (m, 1H), 7.51-7.46 (m, 2H), 6.71-6.69 (m, 1H), 2.44 (t, 3H). |
| 123 | 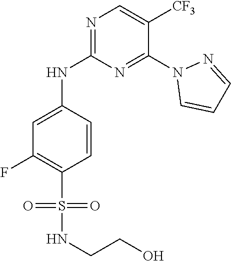 [SMILES: O=S(=O)(NCCO)c1ccc(Nc2ncc(C(F)(F)F)c(-n3cccn3)n2)cc1F] | MS (ES-API positive): 447.2 (M + H)⁺. ¹H NMR (400 MHz, DMSO-d₆) δ 11.02 (s, 1H), 9.05 (s, 1H), 8.60 (d, J = 2.8 Hz, 1H), 8.00 (d, J = 1.5 Hz, 1H), 7.90 (d, J = 13.0 Hz, 1H), 7.84-7.71 (m, 2H), 7.67 (dd, J = 8.7, 2.0 Hz, 1H), 6.72 (t, J = 2.2 Hz, 1H), 4.71 (t, J = 5.6 Hz, 1H), 3.49-3.41 (m, 2H), 2.89 (q, J = 5.9 Hz, 2H). |
| 124 | 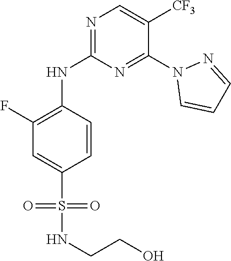 [SMILES: O=S(=O)(NCCO)c1ccc(Nc2ncc(C(F)(F)F)c(-n3cccn3)n2)c(F)c1] | MS (ES-API positive): 447.2 (M + H)⁺. ¹H NMR (400 MHz, DMSO-d₆) δ 10.50 (s, 1H), 8.96 (s, 1H), 8.47 (d, J = 2.8 Hz, 1H), 8.04 (t, J = 8.0 Hz, 1H), 7.96 (d, J = 1.6 Hz, 1H), 7.76-7.65 (m, 3H), 6.68-6.65 (m, 1H), 4.76 (s, 1H), 3.52-3.38 (m, 2H), 2.84 (q, J = 6.0 Hz, 2H). |
| 125 | 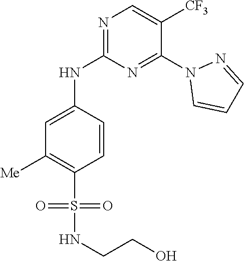 [SMILES: Cc1cc(Nc2ncc(C(F)(F)F)c(-n3cccn3)n2)ccc1S(=O)(=O)NCCO] | MS (ES-API positive): 443.2 (M + H)⁺. ¹H NMR (400 MHz, DMSO-d₆) δ 10.77 (s, 1H), 8.99 (s, 1H), 8.59 (d, J = 2.8 Hz, 1H), 7.98 (d, J = 1.6 Hz, 1H), 7.89-7.75 (m, 2H), 7.73 (d, J = 2.1 Hz, 1H), 7.55 (t, J = 5.8 Hz, 1H), 6.70 (dd, J = 2.7, 1.6 Hz, 1H), 4.69 (t, J = 5.6 Hz, 1H), 3.38-3.32 (m, 2H), 2.81 (q, J = 6.2 Hz, 2H), 2.57 (s, 3H). |
| 126 | 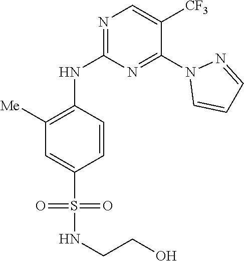 [SMILES: Cc1cc(S(=O)(=O)NCCO)ccc1Nc1ncc(C(F)(F)F)c(-n2cccn2)n1] | MS (ES-API positive): 443.2 (M + H)⁺. ¹H NMR (400 MHz, DMSO-d₆) δ 10.06 (s, 1H), 8.89 (s, 1H), 8.38 (s, 1H), 7.94 (dd, J = 1.7, 0.7 Hz, 1H), 7.78 (d, J = 8.4 Hz, 1H), 7.70 (d, J = 2.2 Hz, 1H), 7.65 (dd, J = 8.3, 2.2 Hz, 1H), 7.55 (d, J = 5.7 Hz, 1H), 6.64 (dd, J = 2.8, 1.6 Hz, 1H), 4.73 (t, J = 5.6 Hz, 1H), 3.41-3.37 (m, 2H), 2.81 (q, J = 5.9 Hz, 2H), 2.34 (s, 3H). |
| 127 | 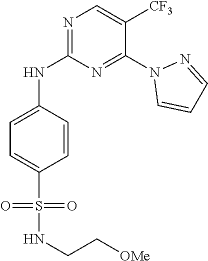 [SMILES: COCCNS(=O)(=O)c1ccc(Nc2ncc(C(F)(F)F)c(-n3cccn3)n2)cc1] | MS (ES-API positive): 443.0 (M + H)⁺. ¹H NMR (400 MHz, DMSO-d₆) δ 10.85 (s, 1H), 9.00 (s, 1H), 8.61 (d, J = 2.6 Hz, 1H), 7.98 (s, 1H), 7.94 (d, J = 8.7 Hz, 2H), 7.80 (d, J = 8.7 Hz, 2H), 7.61 (t, J = 6.0 Hz, 1H), 6.73-6.67 (m, 1H), 3.30 (t, J = 5.8 Hz, 2H), 3.17 (s, 3H), 2.90 (q, J = 5.8 Hz, 2H). |
| 128 | 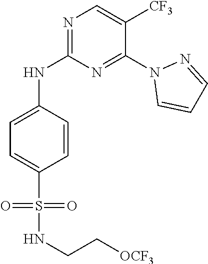 [SMILES: O=S(=O)(NCCOC(F)(F)F)c1ccc(Nc2ncc(C(F)(F)F)c(-n3cccn3)n2)cc1] | MS (ES-API positive): 497.0 (M + H)⁺. ¹H NMR (400 MHz, DMSO-d₆) δ 10.87 (s, 1H), 9.01 (s, 1H), 8.62 (d, J = 2.6 Hz, 1H), 8.01-7.93 (m, 3H), 7.90 (s, 1H), 7.82 (d, J = 8.8 Hz, 2H), 6.71 (d, J = 1.7, 2.6 Hz, 1H), 4.05 (t, J = 5.2 Hz, 2H), 3.09 (t, J = 5.1 Hz, 2H). |
| 129 | 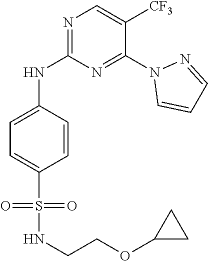 [SMILES: O=S(=O)(NCCOC1CC1)c1ccc(Nc2ncc(C(F)(F)F)c(-n3cccn3)n2)cc1] | MS (ES-API positive): 469.1 (M + H)⁺. ¹H NMR (400 MHz, DMSO-d₆) δ 10.84 (s, 1 H), 9.00 (s, 1 H), 8.61 (d, J = 2.62 Hz, 1 H), 7.98 (d, J = 0.95 Hz, 1 H), 7.94 (d, J = 8.82 Hz, 2 H), 7.79 (d, J = 8.82 Hz, 2 H), 7.58 (t, J = 5.90 Hz, 1 H), 6.70 (dd, J = 2.62, 1.67 Hz, 1 H), 3.39 (t, J = 5.84 Hz, 2 H), 3.20-3.19 (m, 1 H), 2.90 (q, J = 5.88 Hz, 2 H), 0.44-0.30 (m, 4 H). |
| 130 | 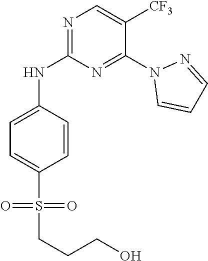 [SMILES: O=S(=O)(CCCO)c1ccc(Nc2ncc(C(F)(F)F)c(-n3cccn3)n2)cc1] | MS (ES-API positive): 428.3 (M + H)⁺. ¹H NMR (400 MHz, DMSO-d₆) δ 10.94 (s, 1H), 9.01 (s, 1H), 8.63 (d, J = 2.6 Hz, 1H), 8.07-7.94 (m, 3H), 7.88 (d, J = 8.8 Hz, 2H), 6.70 (d, J = 2.5 Hz, 1H), 3.41 (t, J = 6.2 Hz, 2H), 3.33-3.14 (m, 2H), 1.76-1.61 (m, 2H). |
| 131 | 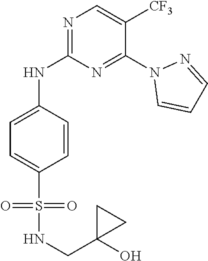 [SMILES: O=S(=O)(NCC1(O)CC1)c1ccc(Nc2ncc(C(F)(F)F)c(-n3cccn3)n2)cc1] | MS (ES-API positive): 455.2 (M + H)⁺. ¹H NMR (400 MHz, DMSO-d₆) δ 10.85 (s, 1H), 9.00 (s, 1H), 8.61 (d, J = 2.8 Hz, 1H), 7.99 (s, 1H), 7.94 (s, 1H), 7.92 (s, 1H), 7.81 (s, 1H), 7.79 (s, 1H), 7.55 (t, J = 6.4 Hz, 1H), 6.70 (dd, J = 2.8, 1.6 Hz, 1H), 5.35 (s, 1H), 2.85 (d, J = 5.6 Hz, 2H), 0.57-0.37 (m, 4H). |
| 132 | 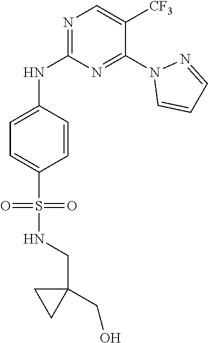 [SMILES: O=S(=O)(NCC1(CO)CC1)c1ccc(Nc2ncc(C(F)(F)F)c(-n3cccn3)n2)cc1] | MS (ES-API positive): 469.2 (M + H)⁺. ¹H NMR (400 MHz, Methanol-d₄) δ 8.91 (s, 1H), 8.60 (dd, J = 2.8, 0.8 Hz, 1H), 8.00-7.93 (m, 2H), 7.89- 7.83 (m, 3H), 6.63 (dd, J = 2.8, 1.6 Hz, 1H), 3.44 (s, 2H), 2.91 (s, 2H), 0.47-0.39 (m, 4H). |
| 133 | 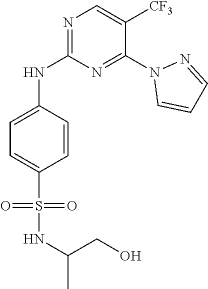 [SMILES: CC(CO)NS(=O)(=O)c1ccc(Nc2ncc(C(F)(F)F)c(-n3cccn3)n2)cc1] | MS (ES-API positive): 443.1 (M + H)⁺. ¹H NMR (400 MHz, DMSO-d₆) δ 10.85 (s, 1H), 9.00 (s, 1H), 8.61 (d, J = 2.6 Hz, 1H), 7.98 (d, J = 1.1 Hz, 1H), 7.94 (d, J = 8.8 Hz, 2H), 7.82 (d, J = 8.8 Hz, 2H), 7.41 (d, J = 6.8 Hz, 1H), 6.70 (d, J = 1.7, 2.6 Hz, 1H), 3.36-3.23 (m, 1H), 3.15-3.03 (m, 2H), 0.90 (d, J = 6.1 Hz, 3H). |
| 134 | 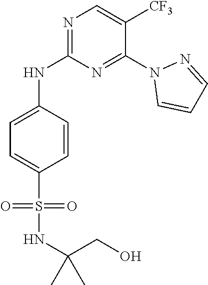 [SMILES: CC(C)(CO)NS(=O)(=O)c1ccc(Nc2ncc(C(F)(F)F)c(-n3cccn3)n2)cc1] | MS (ES-API positive): 457.1 (M + H)⁺. ¹H NMR (400 MHz, DMSO-d₆) δ 10.82 (s, 1H), 9.00 (s, 1H), 8.61 (d, J = 2.6 Hz, 1H), 7.98 (d, J = 1.1 Hz, 1H), 7.95-7.87 (m, 2H), 7.82 (d, J = 8.9 Hz, 2H), 7.20 (s, 1H), 6.70 (d, J = 2.7 Hz, 1H), 3.19 (s, 2H), 1.02 (s, 6H). |
| 135 | 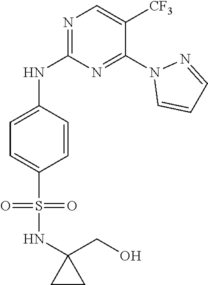 [SMILES: O=S(=O)(NC1(CO)CC1)c1ccc(Nc2ncc(C(F)(F)F)c(-n3cccn3)n2)cc1] | MS (ES-API positive): 455.0 (M + H)⁺. ¹H NMR (400 MHz, DMSO-d₆) δ 10.85 (s, 1H), 9.00 (s, 1H), 8.61 (d, J = 2.5 Hz, 1H), 8.02 (s, 1H), 7.98 (d, J = 1.0 Hz, 1H), 7.93 (d, J = 8.8 Hz, 2H), 7.77 (d, J = 8.8 Hz, 2H), 6.70 (d, J = 1.7, 2.6 Hz, 1H), 3.28 (s, 2H), 0.60-0.52 (m, 2H), 0.52-0.45 (m, 2H). |
| 136 | 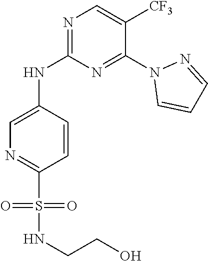 [SMILES: O=S(=O)(NCCO)c1ccc(Nc2ncc(C(F)(F)F)c(-n3cccn3)n2)cn1] | MS (ES-API positive): 430.1 (M + H)⁺. ¹H NMR (500 MHz, DMSO-d₆) δ 11.01 ( s, 1H), 9.12- 8.91 (m, 2H), 8.60 (d, J = 2.6 Hz, 1H), 8.43 (d, J = 2.4 Hz, 1H), 4.66 (t, J = 5.6 Hz, 1H), 3.37 (q, J = 6.4 Hz, 2H), 2.98- 2.92 (m, 2H). |
| 137 | 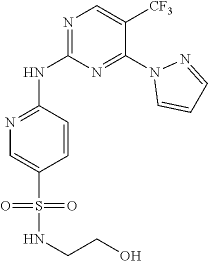 [SMILES: O=S(=O)(NCCO)c1ccc(Nc2ncc(C(F)(F)F)c(-n3cccn3)n2)nc1] | MS (ES-API positive): 430.1 (M + H)⁺. ¹H NMR (400 MHz, DMSO-d₆) δ 11.30 (s, 1H), 9.06 (s, 1H), 8.69 (d, J = 2.5, 2H), 8.37 (d, J = 8.9 Hz, 1H), 8.22 (d, J = 2.4 Hz, 1H), 8.00 (d, J = 1.1 Hz, 1H), 7.72 (t, J = 5.9 Hz, 1H), 6.71 (d, J = 2.5 Hz, 1H), 4.83-4.56 (m, 1H), 3.41- 3.37 (m, 2H), 2.86 (q, J = 6.1 Hz, 2H). |
| 138 | 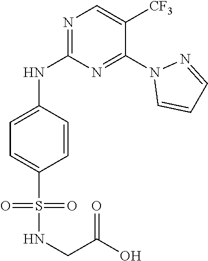 [SMILES: O=C(O)CNS(=O)(=O)c1ccc(Nc2ncc(C(F)(F)F)c(-n3cccn3)n2)cc1] | MS (ES-API positive): 443.0 (M + H)⁺. ¹H NMR (400 MHz, Methanol-d₄) δ 8.90 (s, 1H), 8.59 (d, J = 2.7 Hz, 1H), 7.95 (d, J = 8.8 Hz, 2H), 7.85 (dt, J = 6.1, 2.2 Hz, 3H), 6.62 (dd, J = 2.8, 1.6 Hz, 1H), 3.66 (s, 2H). |

### Example A: Cyclin-Dependent Kinase Activity Assay

Cyclin-dependent kinase activity assay was measured by continuous ATP coupling assay in which ADP generated by kinase catalyzed phosphorylation was converted back to ATP by consuming phosphoenolpyruvate (PEP) and NADH catalyzed by pyruvate kinase/lactate dehydrogenase (PK/LDH). The assay compositions (see, Table 1) consist of one of the kinases, including CDK1/cyclinB1, CDK2/cyclinE1, CDK4/cyclinD3, and CDK6/cyclinD3 at the designated concentration, ATP at the corresponding Kₘ of the kinase, copeptide and the ATP coupling system of PEP, NADH and PK/LDH. The assay was conducted in a reaction buffer of 20 mM Tris (pH 7.5) with 50 mM NaCl, 0.5 mM DTT and 0.04% BSA, and results were monitored continuously by fluorescence decrease of NADH with excitation at 340 nm and emission at 460 nm. Linear regression of the resulting reaction time traces gave reaction velocities.

For inhibition assay, compounds were first serial diluted in a 1:3 ratio from 10 μM to 0.05 nM using 10× reaction buffer. One of the CDK kinase was then introduced to compounds solution and incubated for 15 min. Finally, the compositions of ATP coupling system were introduced to the resulting solution to initiate the reaction. The final reaction mixture was in 1× reaction assay buffer with 1% DMSO. The half-maximal inhibition concentrations (IC₅₀) for compounds were determined by fitting the plots of reaction velocity against compound concentration with 4-variable IC₅₀ equation. The inhibition constants (Kᵢ) were determined by fitting the same plots with quadratic/Morrison equation. Data were analyzed using commercially available software Graphpad Prism.

TABLE 1
CDK Activity Assay Compositions and Conditions

| | | CDK1- | CDK2- | CDK4- | CDK6- |
|---|---|---|---|---|---|
| | | cyclinB1 | cyclinE1 | cyclinD3 | cyclinD3 |
| Kinase | Source | Carna Biosciences | | | |
| | CAS No. | 04-102 | 04-165 | 04-105 | 04-107 |
| | Conc. (nM) | 10 | 5 | 60 | 25 |
| ATP conc. (μM) | | 200 | 300 | 500 | 500 |
| Copeptide | Name | Histone1 | Rb | Rb | Rb |
| | Sequence | GGGPATPKKAKKL | INGSPRTPRRGQNR | INGSPRTPRRGQNR | INGSPRTPRRGQNR |
| | | (SEQ ID NO: 1) | (SEQ ID NO: 2) | (SEQ ID NO 2:) | (SEQ ID NO: 2) |
| | Conc. (μM) | 100 | 100 | 500 | 500 |

| Buffer | 20 mM Tris, 50 mM NaCl, 0.5 mM DTT, 0.04% BSA | |
|---|---|---|
| Coupling | MgCl₂ | 2 mM |
| enzyme | PEP | 2 mM |
| system | NADH | 0.14 mM |
| | PK/LDH | 0.25 U |

Table 2 below provides CDK inhibitory activities of representative compounds. Exemplary results are presented as calculated IC₅₀ values.
A: IC₅₀≤10 nM
B: IC₅₀>10 nM and IC₅₀<100 nM
C: IC₅₀≥100 nM

TABLE 2
Representative CDK Inhibitory Activities

| Example | CDK1 | CDK2 | CDK4 | CDK6 |
|---|---|---|---|---|
| No. | IC₅₀ (nM) | IC₅₀ (nM) | IC₅₀ (nM) | IC₅₀ (nM) |
| 1 | C | B | B | B |
| 2 | B | A | B | B |
| 3 | B | B | B | C |
| 4 | C | B | C | C |
| 5 | B | A | B | B |
| 6 | B | A | B | B |
| 7 | B | A | B | B |
| 8 | C | C | B | B |
| 9 | C | C | C | C |
| 10 | B | A | B | B |
| 11 | B | A | A | B |
| 12 | C | B | B | C |
| 13 | B | A | B | B |
| 14 | C | B | B | C |
| 15 | B | A | B | B |
| 16 | C | A | C | C |
| 17 | C | A | C | C |
| 18 | C | A | C | C |
| 19 | C | A | B | B |
| 20 | C | B | C | C |
| 21 | C | B | C | C |
| 22 | C | B | C | C |
| 23 | C | C | C | C |
| 24 | C | B | C | C |
| 25 | C | A | C | C |
| 26 | C | B | C | C |
| 27 | C | C | C | C |
| 28 | C | B | C | C |
| 29 | C | C | C | C |
| 30 | C | B | C | C |
| 31 | C | C | C | C |
| 32 | C | C | C | C |
| 33 | C | C | C | C |
| 34 | C | C | C | C |
| 35 | C | A | B | C |
| 36 | B | A | C | C |
| 37 | C | A | B | B |
| 38 | C | B | C | C |
| 39 | C | C | C | C |
| 40 | C | A | C | C |
| 41 | C | B | C | C |
| 42 | C | C | C | C |
| 43 | B | A | B | B |
| 44 | C | B | C | C |
| 45 | C | B | C | C |
| 46 | C | A | B | C |
| 47 | B | B | C | C |
| 48 | C | B | A | B |
| 49 | B | B | B | B |
| 50 | C | A | B | B |
| 51 | C | B | C | C |
| 52 | C | B | C | B |
| 53 | C | B | C | C |
| 54 | C | B | C | C |
| 55 | C | C | C | C |
| 56 | C | C | C | C |
| 57 | C | B | C | C |
| 58 | B | A | B | B |
| 59 | B | A | B | B |
| 60 | B | B | B | C |
| 61 | C | A | B | C |
| 62 | C | B | B | C |
| 63 | C | B | B | B |
| 64 | C | A | B | B |
| 65 | B | A | B | B |
| 66 | C | B | B | B |
| 67 | C | B | C | C |
| 68 | B | A | B | B |
| 69 | C | B | C | C |
| 70 | C | B | C | C |
| 71 | B | A | A | B |
| 72 | B | A | B | C |
| 73 | C | B | B | B |
| 74 | C | B | B | C |
| 75 | C | B | B | C |
| 76 | C | B | B | C |
| 77 | B | B | B | C |
| 78 | C | A | B | B |
| 79 | B | A | B | B |
| 80 | B | A | B | B |
| 81 | B | B | B | B |
| 82 | B | A | B | C |
| 83 | C | A | C | C |
| 84 | C | B | C | C |
| 85 | B | A | B | C |
| 86 | C | B | C | C |
| 87 | C | B | C | C |
| 88 | C | B | C | C |
| 89 | C | A | B | B |
| 90 | C | B | B | B |
| 91 | B | A | B | B |
| 92 | B | A | A | B |
| 93 | B | A | B | C |
| 94 | B | A | B | C |
| 95 | B | A | B | C |
| 96 | B | A | B | B |
| 97 | B | A | B | C |
| 98 | C | B | C | C |
| 99 | C | B | C | C |
| 100 | B | A | C | C |
| 101 | C | B | C | C |
| 102 | C | A | B | B |
| 103 | B | A | B | B |
| 104 | C | B | B | C |
| 105 | B | B | B | B |
| 106 | C | B | C | C |
| 107 | B | A | B | B |
| 108 | C | B | B | B |
| 109 | C | B | B | B |
| 110 | C | A | C | C |
| 111 | C | A | C | C |
| 112 | C | A | C | C |
| 113 | C | A | C | C |
| 114 | C | B | C | C |
| 115 | C | B | C | C |
| 116 | C | B | C | C |
| 117 | B | A | C | C |
| 118 | C | C | C | C |
| 119 | C | B | C | B |
| 120 | C | B | C | C |
| 121 | C | B | C | C |
| 122 | C | C | C | C |
| 123 | C | B | C | C |
| 124 | C | A | C | C |
| 125 | C | B | C | C |
| 126 | C | A | C | C |
| 127 | C | B | C | C |
| 128 | C | C | C | C |
| 129 | C | C | C | C |
| 130 | C | B | C | C |
| 131 | C | A | C | C |
| 132 | C | B | C | C |
| 133 | B | A | C | C |
| 134 | C | A | C | C |
| 135 | C | A | C | C |
| 136 | C | A | C | C |
| 137 | C | C | C | C |
| 138 | C | A | C | C |

1. A compound of Formula (I), 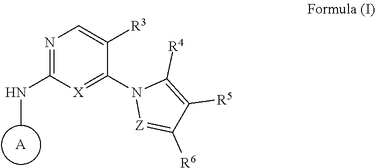
wherein
X is CH or N;
Z is CH or N;
ring A is aryl, heteroaryl, cycloalkyl or heterocyclyl, or aryl-fused heterocyclyl, each of which is optionally and independently substituted with one R¹ and one to four R²; wherein a carbon atom of the aryl-fused heterocyclyl is optionally substituted with one oxo, and a heteroatom of the aryl-fused heterocyclyl is optionally substituted with one or two oxo;
R¹ is —S(O)₂—R¹⁰, —NH—S(O)₂—R¹⁰ or —SF₅;
each R² is independently H, alkyl, —CN, cyanoalkyl, amino, alkylamino, dialkylamino, —NO₂, halo, haloalkyl, hydroxyl, hydroxyalkyl, alkoxyl, haloalkoxy, (haloalkoxy)alkyl, alkoxyalkyl, cycloalkyl or heterocyclyl; wherein the cycloalkyl or heterocyclyl is optionally substituted with one or more R⁷;
R³ is H, halo, alkyl, haloalkyl, hydroxyl, hydroxyalkyl, alkoxyl, alkoxyalkyl, alkenyl, alkynyl, —CN or cyanoalkyl;
R⁴ and R⁶ are each independently H, alkyl, halo, haloalkyl, hydroxyl, hydroxyalkyl, alkoxyl, haloalkoxy, (haloalkoxy)alkyl, alkoxyalkyl, amino, alkylamino, dialkylamino, -alk-N—R⁹R⁹, —CN, cyanoalkyl, cycloalkyl or heterocyclyl; wherein the cycloalkyl or heterocyclyl is optionally substituted with one or more R⁷;
R⁵ is H, alkyl, halo, haloalkyl, hydroxyl, hydroxyalkyl, alkoxyl, haloalkoxy, (haloalkoxy)alkyl, alkoxyalkyl, —CN, cyanoalkyl, amino, alkylamino, dialkylamino, -alk-N—R⁹R⁹, aryl, heteroaryl, cycloalkyl, cycloalkenyl, heterocyclyl, -alk-cycloalkyl, -alk-heterocyclyl, —O-cycloalkyl, or —O— heterocyclyl, wherein the aryl, heteroaryl, cycloalkyl, cycloalkenyl, heterocyclyl, -alk-cycloalkyl, -alk-heterocyclyl, —O-cycloalkyl or —O-heterocyclyl is optionally substituted with one or more R⁷;
each R⁷ is independently alkyl, halo, haloalkyl, hydroxyl, hydroxyalkyl, alkoxyl, haloalkoxy, (haloalkoxy)alkyl, alkoxyalkyl, amino, alkylamino, dialkylamino, —CN, cyanoalkyl, oxo (═O), R⁸—S(O)₂—, R⁸—O—C(═O)—, R⁸—C(═O)—O—, R⁸—C(═O)—, cycloalkyl or heterocyclyl; wherein the cycloalkyl or heterocyclyl is optionally further substituted with one or more R⁷;
each R⁸ is independently H, alkyl or —NR⁹R⁹;
each R⁹ is independently H, cycloalkyl, -alk-cycloalkyl, or alkyl;
R¹⁰ is H, alkyl, haloalkyl, hydroxyalkyl, alkoxyalkyl, cycloalkyl, heterocyclyl or —NR¹¹R¹¹; wherein the cycloalkyl or heterocyclyl is optionally substituted with one or more R⁷;
each R¹¹ is independently H, alkyl, cycloalkyl, heterocyclyl, -alk-cycloalkyl, -alk-heterocyclyl, hydroxyalkyl, -alk-O—R¹², -alk-C(O)—OR⁹ or -alk-N—R⁹R⁹; wherein the cycloalkyl, -alk-cycloalkyl, -alk-heterocyclyl, or heterocyclyl is optionally substituted with one or more R⁷;
each R¹² is independently H, alkyl, cycloalkyl, haloalkyl, or heterocyclyl;
the heteroaryl and heterocyclyl in each occurrence independently contain one or more ring-forming heteroatoms each of which is independently oxygen, sulfur or nitrogen;
the heterocyclyl in each occurrence is saturated or partially unsaturated; and
any H in each alkyl is optionally replaced with deuterium (D);
or a pharmaceutically acceptable salt, tautomer, or stereoisomer thereof.

2. The compound of claim 1, wherein ring A is

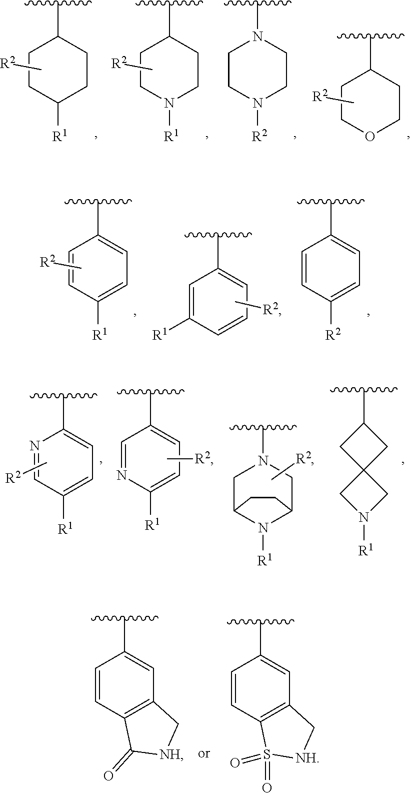

3. The compound of claim 1, wherein the hydroxyalkyl is a linear or branched hydroxyalkyl, or the cyanoalkyl is a linear or branched cyanoalkyl.

4. (canceled)

5. The compound of claim 1, wherein cycloalkyl, cycloalkenyl, or heterocyclyl for ring A is monocyclic or bicyclic.

6. The compound of claim 1, wherein X is N.

7. The compound of claim 1, wherein Z is N.

8. The compound of claim 1, wherein the compound is of Formula (II): 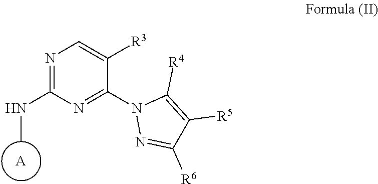
wherein ring A is the same as defined in claim 1.

9. The compound of claim 1, wherein the compound is of Formula (IIa), (IIb), (IIc), (IId) or (IIe): 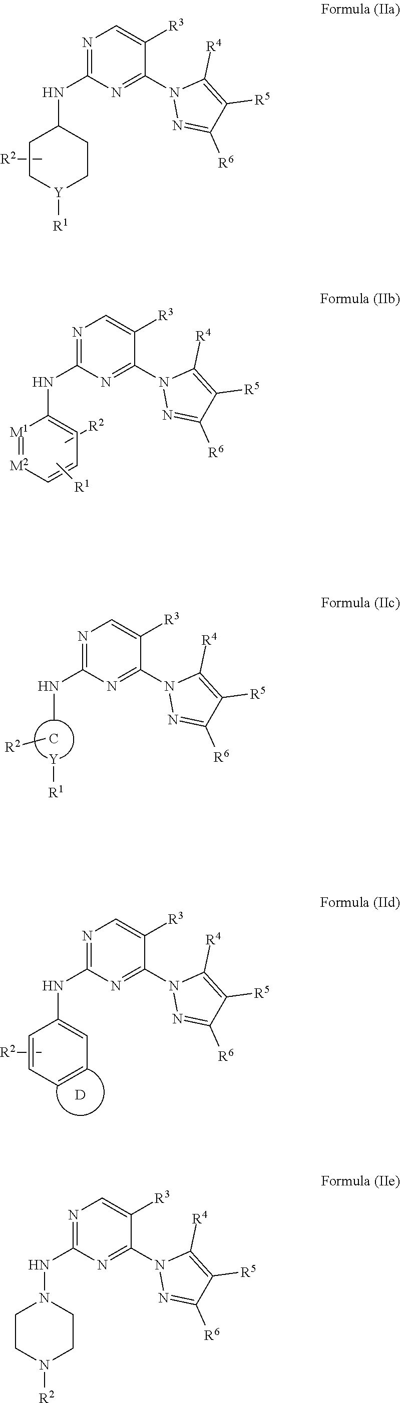
wherein
ring C is a bridged or spiro bicyclic heterocycloalkyl;
ring D is a 4-8 membered cycloalkyl or heterocyclyl fused to the adjacent aryl; optionally, ring D is substituted with one or two oxo;
Y is N, O or CH; and when Y is O, R¹ is absent;
M¹ and M² are each independently N or CH; and
R¹, R², R³, R⁴, R⁵ and R⁶ are the same as claim 1.

10. The compound of claim 1, wherein R¹ is NHR¹¹—S(O)₂—, heterocyclyl-S(O)₂—, C₁₋₆alkyl-S(O)₂—, (C₁₋₆alkyl)₂-N—S(O)₂—, (deuterated C₁₋₆alkyl)₂-N—S(O)₂—, (deuterated C₁₋₆alkyl)-HN—S(O)₂—, C₁₋₆alkyl-S(O)₂—NH—, or —SF₅.

11. The compound of claim 1, wherein R² is H, halo, haloalkyl, hydroxyl, alkyl, cycloalkyl or heterocyclyl; of which cycloalkyl or heterocyclyl is optionally substituted with one or more R⁷.

12. The compound of claim 1, wherein R³ is H, halo, alkyl, haloalkyl, alkynyl, —CN or cyanoalkyl.

13. The compound of claim 1, wherein halo is F, Cl, or Br.

14. The compound of claim 1, wherein the compound is of Formula (III) 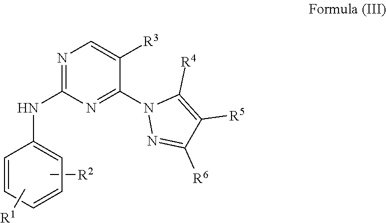
wherein R¹, R², R³, R⁴, R⁵ and R⁶ are the same as defined in claim 1.

15. The compound of claim 1, wherein the compound is selected from the following:

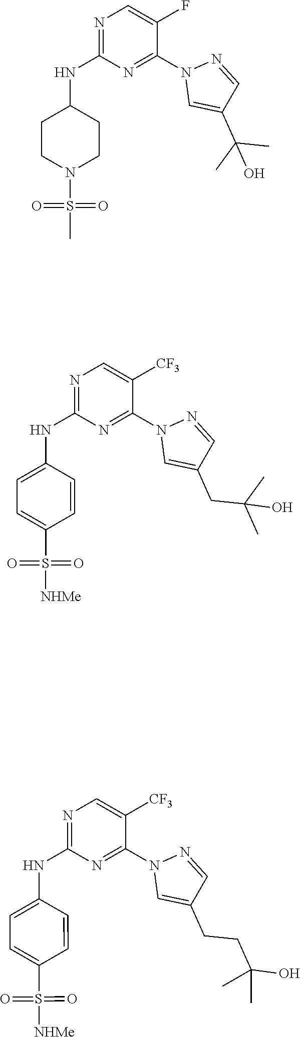 [SMILES: CC(C)(O)c1cnn(-c2nc(NC3CCN(S(C)(=O)=O)CC3)ncc2F)c1 ; CNS(=O)(=O)c1ccc(Nc2ncc(C(F)(F)F)c(-n3cc(CC(C)(C)O)cn3)n2)cc1 ; CNS(=O)(=O)c1ccc(Nc2ncc(C(F)(F)F)c(-n3cc(CCC(C)(C)O)cn3)n2)cc1]

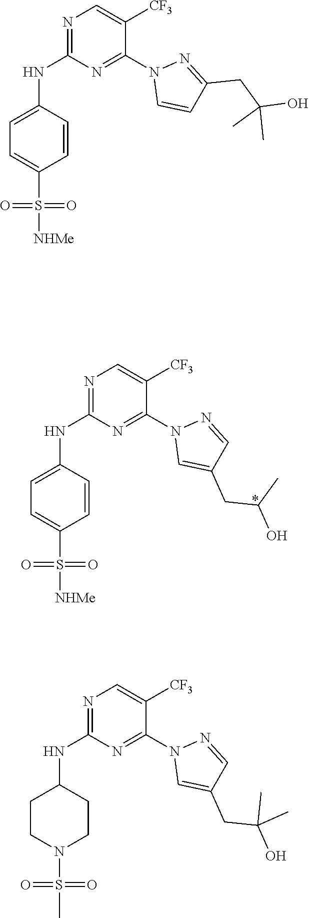 [SMILES: CNS(=O)(=O)c1ccc(Nc2ncc(C(F)(F)F)c(-n3ccc(CC(C)(C)O)n3)n2)cc1 ; CNS(=O)(=O)c1ccc(Nc2ncc(C(F)(F)F)c(-n3cc(CC(C)O)cn3)n2)cc1 ; CC(C)(O)Cc1cnn(-c2nc(NC3CCN(S(C)(=O)=O)CC3)ncc2C(F)(F)F)c1]

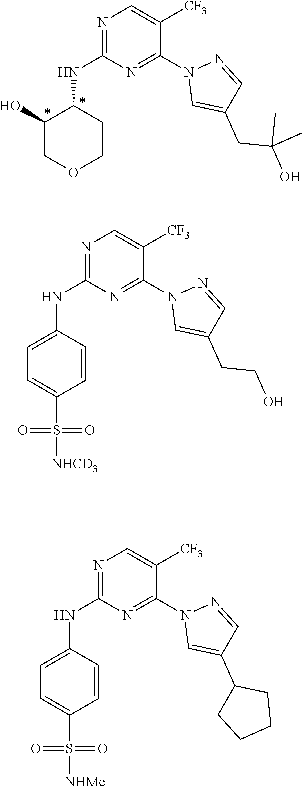

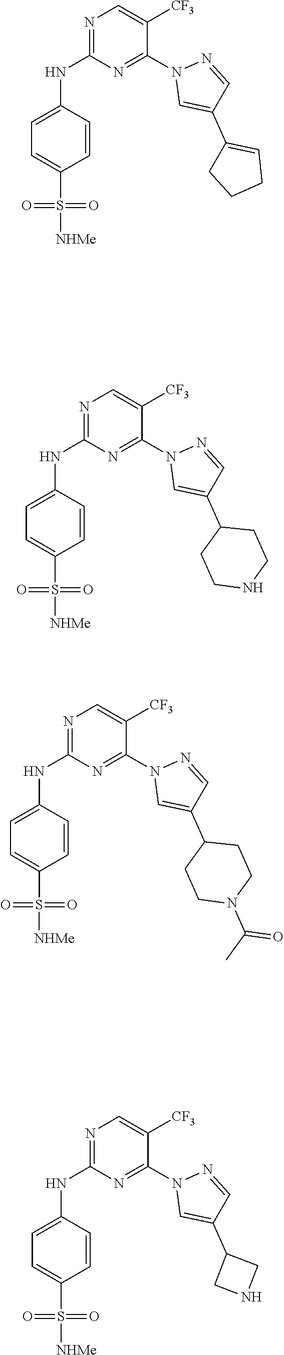 [SMILES: CNS(=O)(=O)c1ccc(Nc2ncc(C(F)(F)F)c(-n3cc(C4=CCCC4)cn3)n2)cc1 ; CNS(=O)(=O)c1ccc(Nc2ncc(C(F)(F)F)c(-n3cc(C4CCNCC4)cn3)n2)cc1 ; CNS(=O)(=O)c1ccc(Nc2ncc(C(F)(F)F)c(-n3cc(C4CCN(C(C)=O)CC4)cn3)n2)cc1 ; CNS(=O)(=O)c1ccc(Nc2ncc(C(F)(F)F)c(-n3cc(C4CNC4)cn3)n2)cc1]

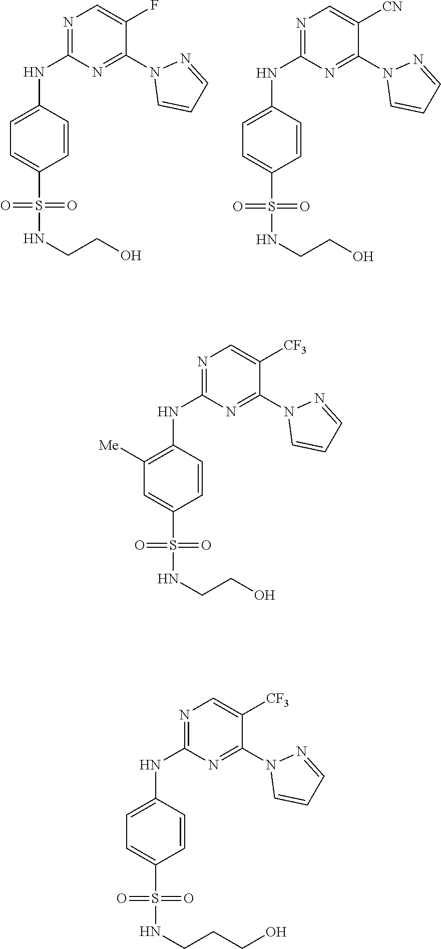 [SMILES: O=S(=O)(NCCO)c1ccc(Nc2ncc(F)c(-n3cccn3)n2)cc1 ; N#Cc1cnc(Nc2ccc(S(=O)(=O)NCCO)cc2)nc1-n1cccn1 ; Cc1cc(S(=O)(=O)NCCO)ccc1Nc1ncc(C(F)(F)F)c(-n2cccn2)n1 ; O=S(=O)(NCCCO)c1ccc(Nc2ncc(C(F)(F)F)c(-n3cccn3)n2)cc1]

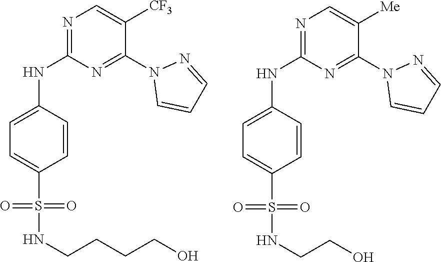 [SMILES: O=S(=O)(NCCCCO)c1ccc(Nc2ncc(C(F)(F)F)c(-n3cccn3)n2)cc1 ; Cc1cnc(Nc2ccc(S(=O)(=O)NCCO)cc2)nc1-n1cccn1]

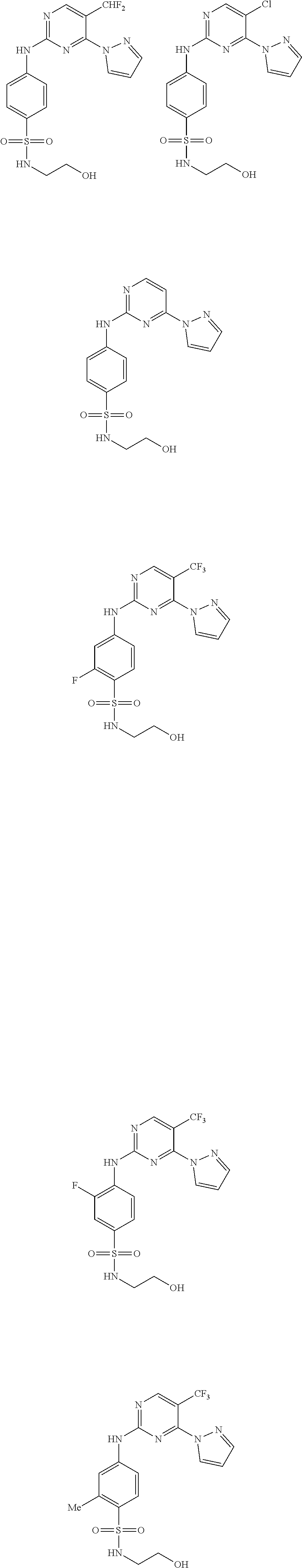 [SMILES: O=S(=O)(NCCO)c1ccc(Nc2ncc(C(F)F)c(-n3cccn3)n2)cc1 ; O=S(=O)(NCCO)c1ccc(Nc2ncc(Cl)c(-n3cccn3)n2)cc1 ; O=S(=O)(NCCO)c1ccc(Nc2nccc(-n3cccn3)n2)cc1 ; O=S(=O)(NCCO)c1ccc(Nc2ncc(C(F)(F)F)c(-n3cccn3)n2)cc1F ; O=S(=O)(NCCO)c1ccc(Nc2ncc(C(F)(F)F)c(-n3cccn3)n2)c(F)c1 ; Cc1cc(Nc2ncc(C(F)(F)F)c(-n3cccn3)n2)ccc1S(=O)(=O)NCCO]

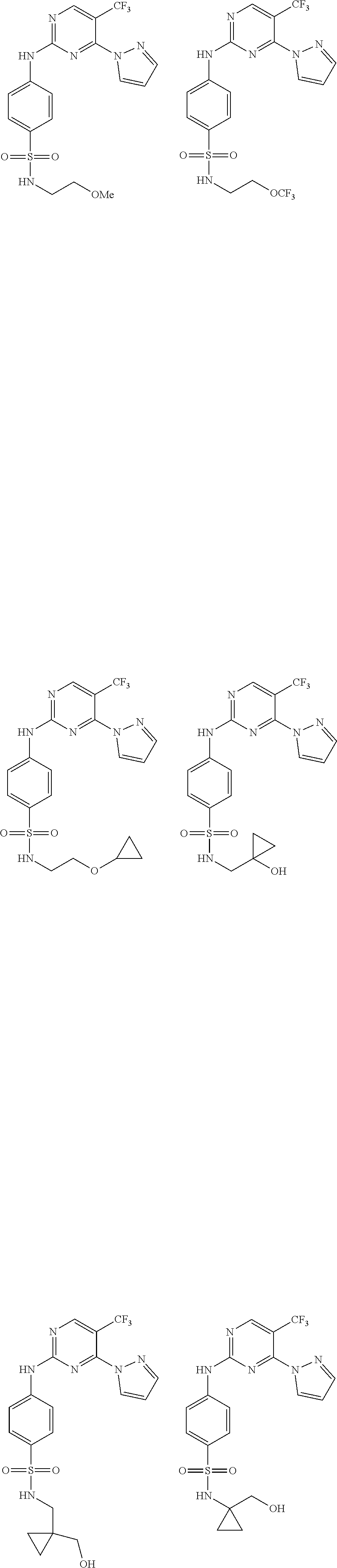 [SMILES: COCCNS(=O)(=O)c1ccc(Nc2ncc(C(F)(F)F)c(-n3cccn3)n2)cc1 ; O=S(=O)(NCCOC(F)(F)F)c1ccc(Nc2ncc(C(F)(F)F)c(-n3cccn3)n2)cc1 ; O=S(=O)(NCCOC1CC1)c1ccc(Nc2ncc(C(F)(F)F)c(-n3cccn3)n2)cc1 ; O=S(=O)(NCC1(O)CC1)c1ccc(Nc2ncc(C(F)(F)F)c(-n3cccn3)n2)cc1 ; O=S(=O)(NCC1(CO)CC1)c1ccc(Nc2ncc(C(F)(F)F)c(-n3cccn3)n2)cc1 ; O=S(=O)(NC1(CO)CC1)c1ccc(Nc2ncc(C(F)(F)F)c(-n3cccn3)n2)cc1]

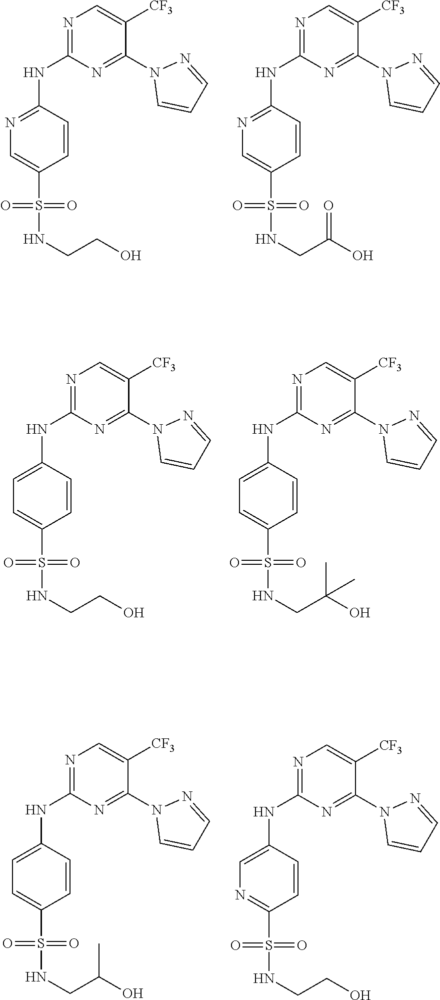 [SMILES: O=S(=O)(NCCO)c1ccc(Nc2ncc(C(F)(F)F)c(-n3cccn3)n2)nc1 ; O=C(O)CNS(=O)(=O)c1ccc(Nc2ncc(C(F)(F)F)c(-n3cccn3)n2)nc1 ; O=S(=O)(NCCO)c1ccc(Nc2ncc(C(F)(F)F)c(-n3cccn3)n2)cc1 ; CC(C)(O)CNS(=O)(=O)c1ccc(Nc2ncc(C(F)(F)F)c(-n3cccn3)n2)cc1 ; CC(O)CNS(=O)(=O)c1ccc(Nc2ncc(C(F)(F)F)c(-n3cccn3)n2)cc1 ; O=S(=O)(NCCO)c1ccc(Nc2ncc(C(F)(F)F)c(-n3cccn3)n2)cn1]

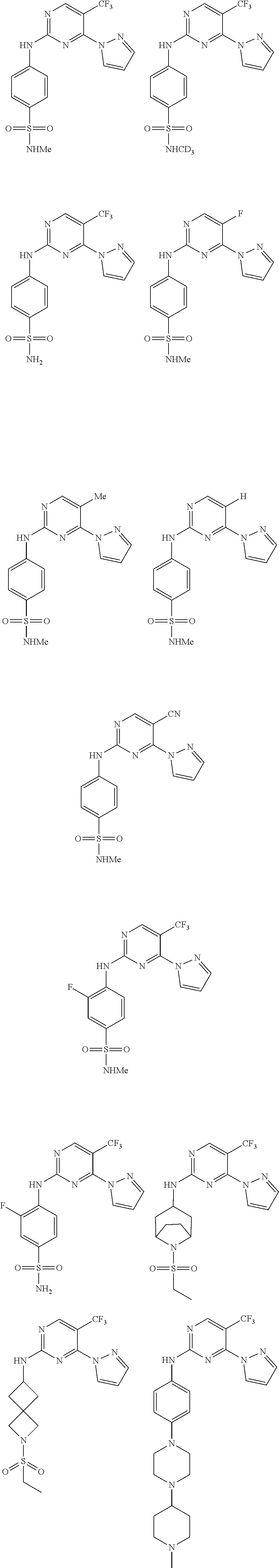

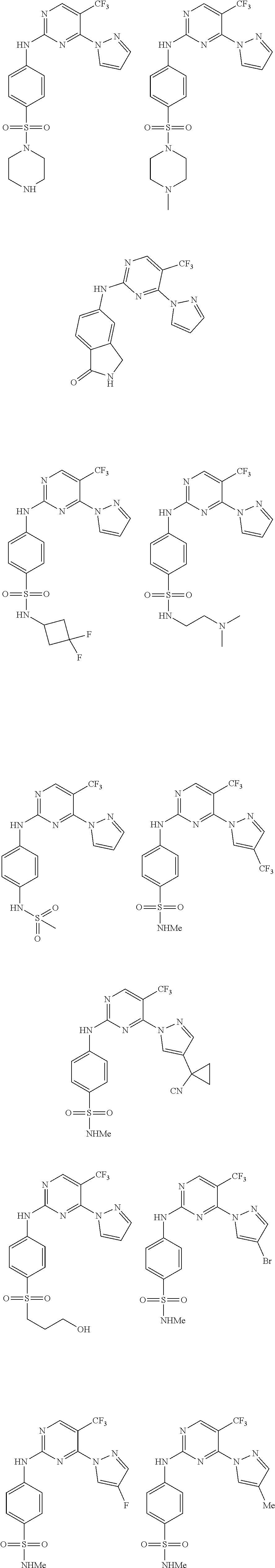

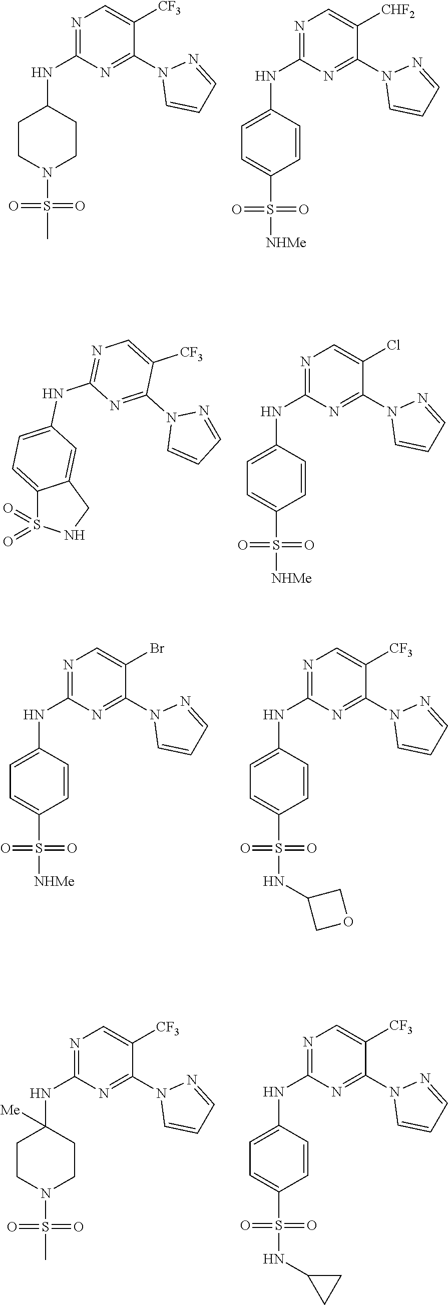

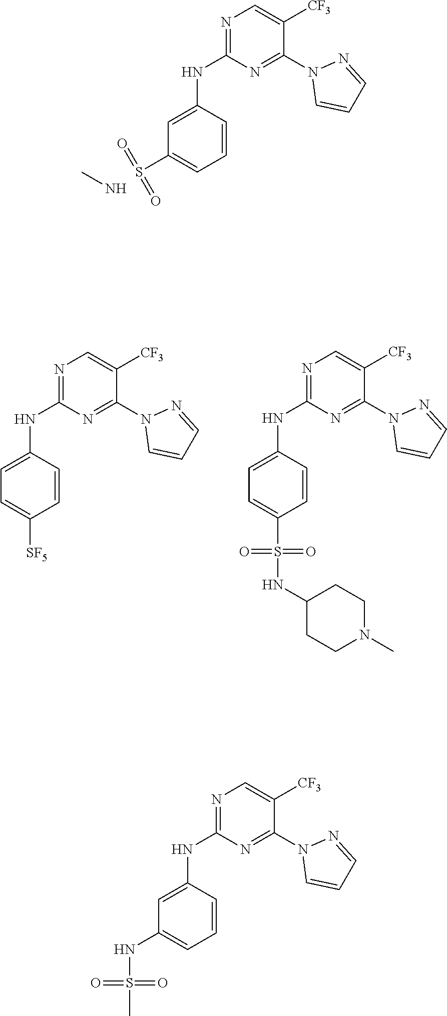

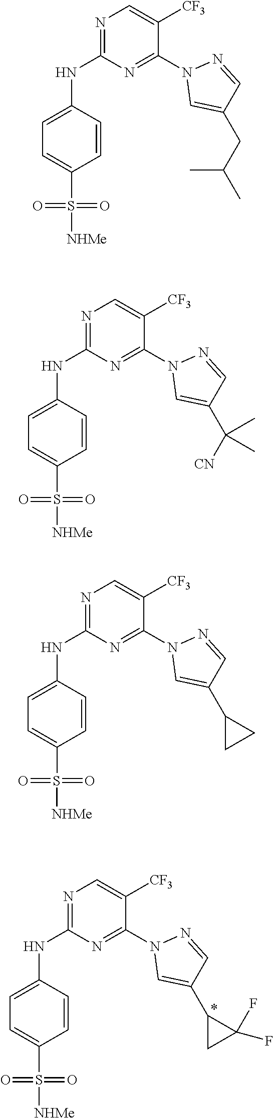

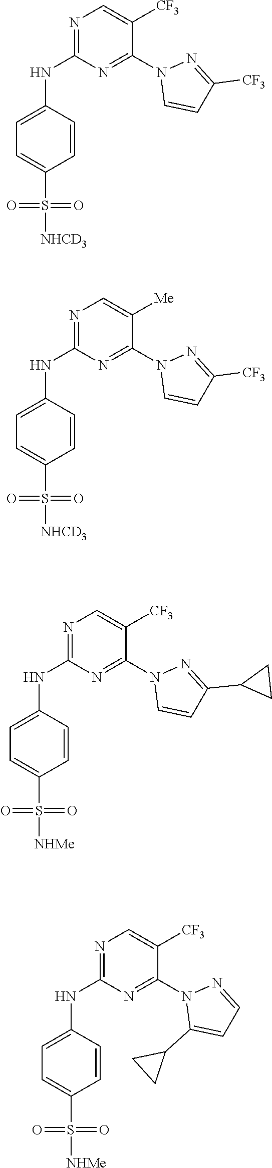

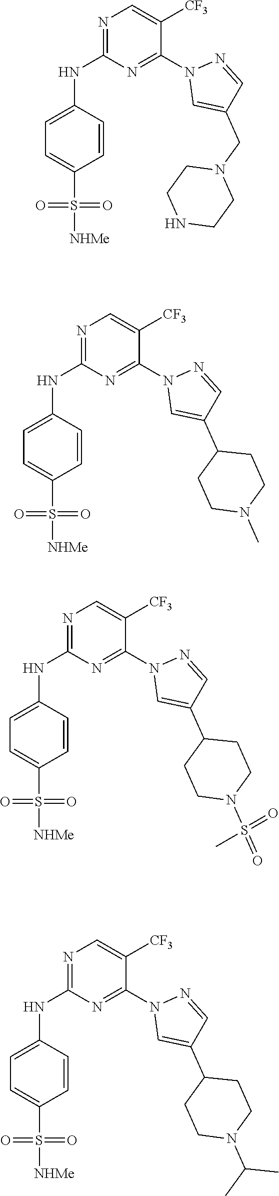 [SMILES: CNS(=O)(=O)c1ccc(Nc2ncc(C(F)(F)F)c(-n3cc(CN4CCNCC4)cn3)n2)cc1 ; CNS(=O)(=O)c1ccc(Nc2ncc(C(F)(F)F)c(-n3cc(C4CCN(C)CC4)cn3)n2)cc1 ; CNS(=O)(=O)c1ccc(Nc2ncc(C(F)(F)F)c(-n3cc(C4CCN(S(C)(=O)=O)CC4)cn3)n2)cc1 ; CNS(=O)(=O)c1ccc(Nc2ncc(C(F)(F)F)c(-n3cc(C4CCN(C(C)C)CC4)cn3)n2)cc1]

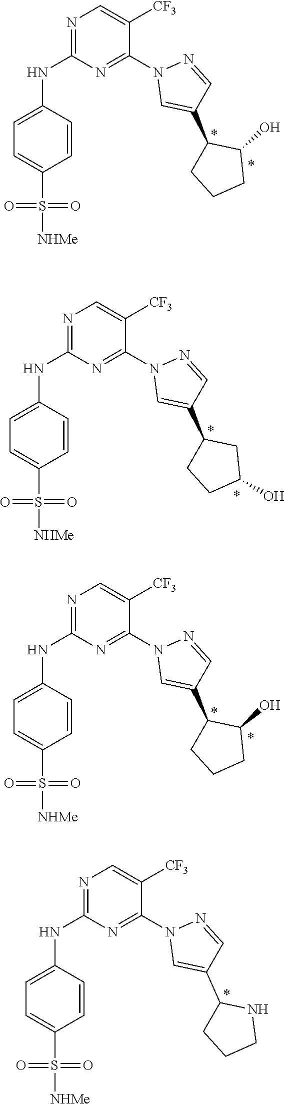 [SMILES: CNS(=O)(=O)c1ccc(Nc2ncc(C(F)(F)F)c(-n3cc([C@@H]4CCC[C@H]4O)cn3)n2)cc1 ; CNS(=O)(=O)c1ccc(Nc2ncc(C(F)(F)F)c(-n3cc([C@@H]4CC[C@@H](O)C4)cn3)n2)cc1 ; CNS(=O)(=O)c1ccc(Nc2ncc(C(F)(F)F)c(-n3cc([C@@H]4CCC[C@@H]4O)cn3)n2)cc1 ; CNS(=O)(=O)c1ccc(Nc2ncc(C(F)(F)F)c(-n3cc(C4CCCN4)cn3)n2)cc1]

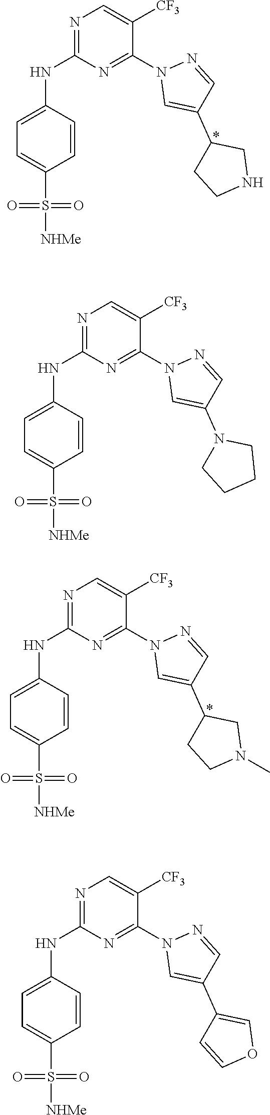 [SMILES: CNS(=O)(=O)c1ccc(Nc2ncc(C(F)(F)F)c(-n3cc(C4CCNC4)cn3)n2)cc1 ; CNS(=O)(=O)c1ccc(Nc2ncc(C(F)(F)F)c(-n3cc(N4CCCC4)cn3)n2)cc1 ; CNS(=O)(=O)c1ccc(Nc2ncc(C(F)(F)F)c(-n3cc(C4CCN(C)C4)cn3)n2)cc1 ; CNS(=O)(=O)c1ccc(Nc2ncc(C(F)(F)F)c(-n3cc(-c4ccoc4)cn3)n2)cc1]

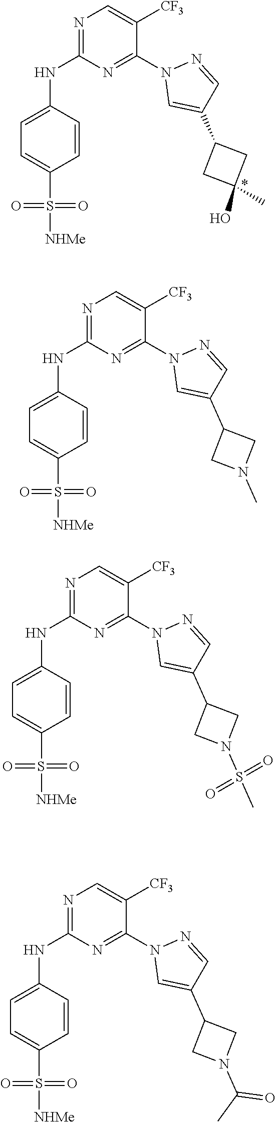 [SMILES: CNS(=O)(=O)c1ccc(Nc2ncc(C(F)(F)F)c(-n3cc([C@H]4C[C@](C)(O)C4)cn3)n2)cc1 ; CNS(=O)(=O)c1ccc(Nc2ncc(C(F)(F)F)c(-n3cc(C4CN(C)C4)cn3)n2)cc1 ; CNS(=O)(=O)c1ccc(Nc2ncc(C(F)(F)F)c(-n3cc(C4CN(S(C)(=O)=O)C4)cn3)n2)cc1 ; CNS(=O)(=O)c1ccc(Nc2ncc(C(F)(F)F)c(-n3cc(C4CN(C(C)=O)C4)cn3)n2)cc1]

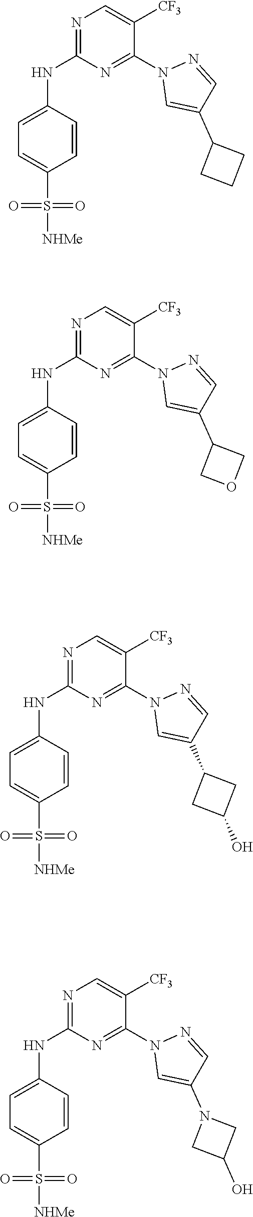 [SMILES: CNS(=O)(=O)c1ccc(Nc2ncc(C(F)(F)F)c(-n3cc(C4CCC4)cn3)n2)cc1 ; CNS(=O)(=O)c1ccc(Nc2ncc(C(F)(F)F)c(-n3cc(C4COC4)cn3)n2)cc1 ; CNS(=O)(=O)c1ccc(Nc2ncc(C(F)(F)F)c(-n3cc([C@H]4C[C@@H](O)C4)cn3)n2)cc1 ; CNS(=O)(=O)c1ccc(Nc2ncc(C(F)(F)F)c(-n3cc(N4CC(O)C4)cn3)n2)cc1]

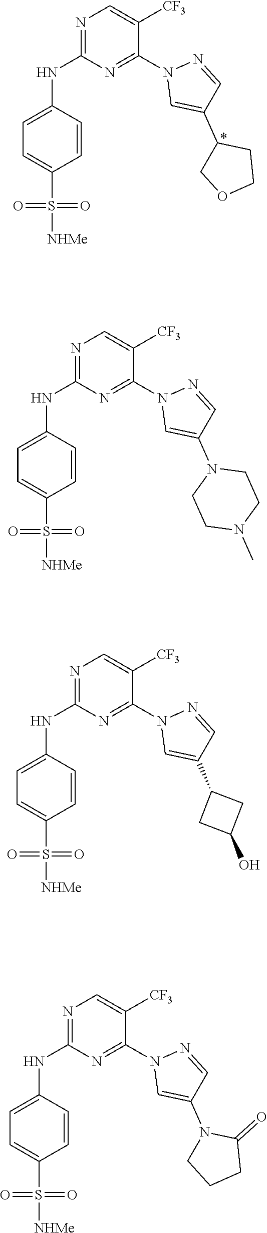 [SMILES: CNS(=O)(=O)c1ccc(Nc2ncc(C(F)(F)F)c(-n3cc(C4CCOC4)cn3)n2)cc1 ; CNS(=O)(=O)c1ccc(Nc2ncc(C(F)(F)F)c(-n3cc(N4CCN(C)CC4)cn3)n2)cc1 ; CNS(=O)(=O)c1ccc(Nc2ncc(C(F)(F)F)c(-n3cc([C@H]4C[C@H](O)C4)cn3)n2)cc1 ; CNS(=O)(=O)c1ccc(Nc2ncc(C(F)(F)F)c(-n3cc(N4CCCC4=O)cn3)n2)cc1]

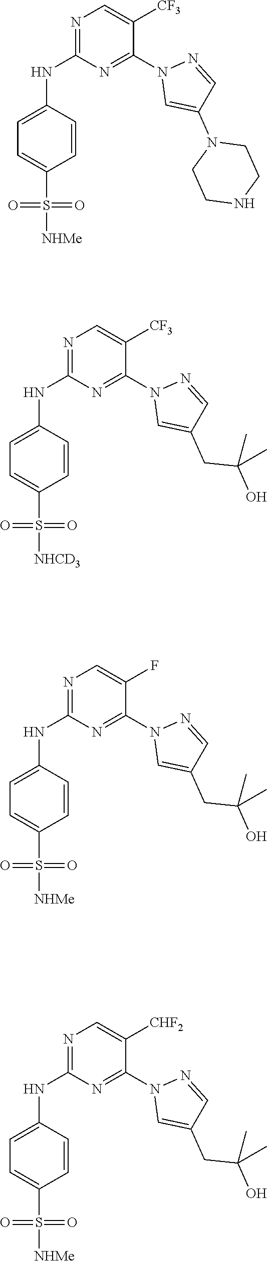

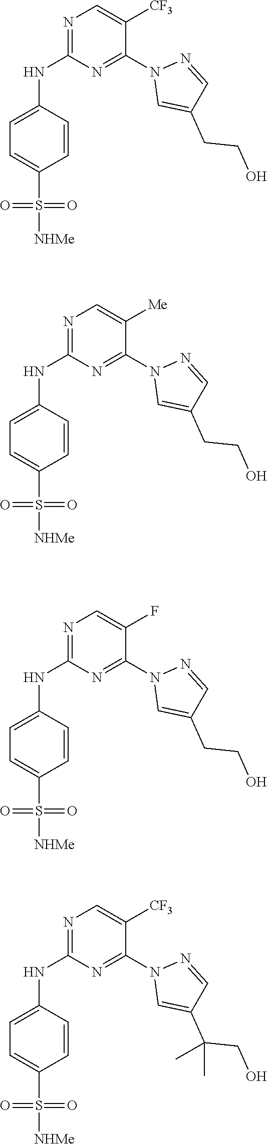 [SMILES: CNS(=O)(=O)c1ccc(Nc2ncc(C(F)(F)F)c(-n3cc(CCO)cn3)n2)cc1 ; CNS(=O)(=O)c1ccc(Nc2ncc(C)c(-n3cc(CCO)cn3)n2)cc1 ; CNS(=O)(=O)c1ccc(Nc2ncc(F)c(-n3cc(CCO)cn3)n2)cc1 ; CNS(=O)(=O)c1ccc(Nc2ncc(C(F)(F)F)c(-n3cc(C(C)(C)CO)cn3)n2)cc1]

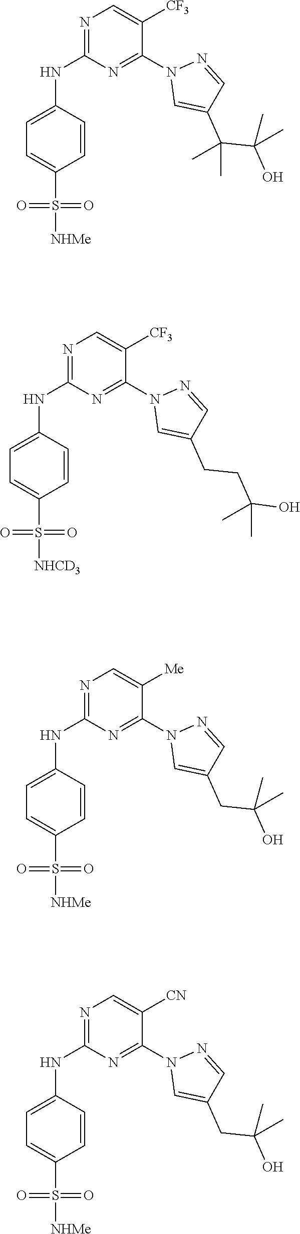

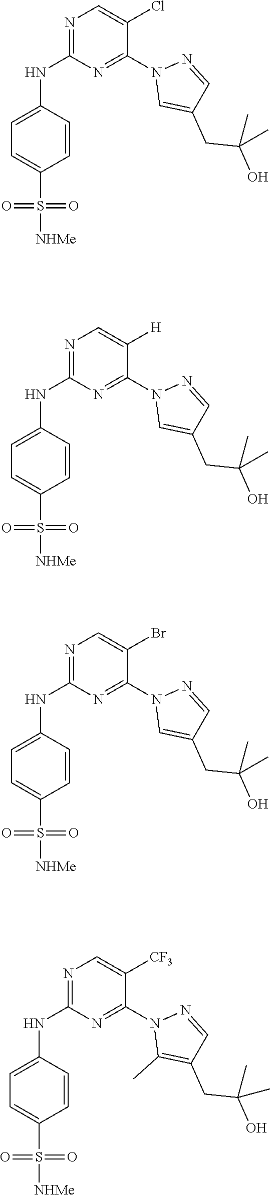 [SMILES: CNS(=O)(=O)c1ccc(Nc2ncc(Cl)c(-n3cc(CC(C)(C)O)cn3)n2)cc1 ; CNS(=O)(=O)c1ccc(Nc2nccc(-n3cc(CC(C)(C)O)cn3)n2)cc1 ; CNS(=O)(=O)c1ccc(Nc2ncc(Br)c(-n3cc(CC(C)(C)O)cn3)n2)cc1 ; CNS(=O)(=O)c1ccc(Nc2ncc(C(F)(F)F)c(-n3ncc(CC(C)(C)O)c3C)n2)cc1]

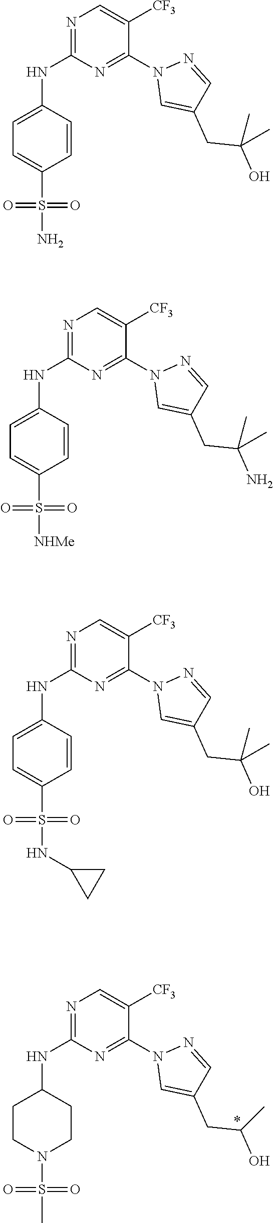 [SMILES: CC(C)(O)Cc1cnn(-c2nc(Nc3ccc(S(N)(=O)=O)cc3)ncc2C(F)(F)F)c1 ; CNS(=O)(=O)c1ccc(Nc2ncc(C(F)(F)F)c(-n3cc(CC(C)(C)N)cn3)n2)cc1 ; CC(C)(O)Cc1cnn(-c2nc(Nc3ccc(S(=O)(=O)NC4CC4)cc3)ncc2C(F)(F)F)c1 ; CC(O)Cc1cnn(-c2nc(NC3CCN(S(C)(=O)=O)CC3)ncc2C(F)(F)F)c1]

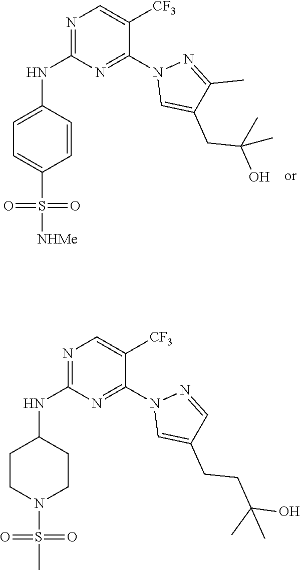 [SMILES: CNS(=O)(=O)c1ccc(Nc2ncc(C(F)(F)F)c(-n3cc(CC(C)(C)O)c(C)n3)n2)cc1 ; "or" ; CC(C)(O)CCc1cnn(-c2nc(NC3CCN(S(C)(=O)=O)CC3)ncc2C(F)(F)F)c1]

16. A pharmaceutical composition comprising a therapeutically effective amount of a compound of claim 1, and a pharmaceutically acceptable carrier or excipient.

17. The pharmaceutical composition of claim 16, further comprising a second therapeutic agent.

18. The pharmaceutical composition of claim 17, wherein the second therapeutic agent is a CDK4/6 inhibitor, estrogen receptor antagonist, hormone therapy, PARP inhibitor, S phase inhibitor, M phase inhibitor or BCL-2 inhibitor.

19. A method for treating a disorder or disease mediated by CDK2 in a subject in need thereof, comprising administering to the subject a therapeutically effective amount of a compound of claim 1.

20. The method of claim 19, wherein the disorder or disease mediated by CDK2 is cancer.

21. The method of claim 20, wherein the cancer is breast cancer, colorectal cancer, lung cancer, ovarian cancer, pancreatic cancer, melanoma, prostate cancer, glioblastoma, or sarcoma.

22. (canceled)
